(19) United States
(12) Patent Application Publication
(10) Pub. No.: US 2023/0366794 A1
(43) Pub. Date: Nov. 16, 2023

(54) PROCESS FOR PREPARING EXTRACELLULAR VESICLES

(71) Applicant: CODIAK BIOSCIENCES, INC., Cambridge, MA (US)

(72) Inventors: Raymond BOURDEAU, Watertown, MA (US); Michael F. DOHERTY, Somerville, MA (US); Aaron NOYES, Melrose, MA (US); Kayla DESANTY, Cambridge, MA (US)

(73) Assignee: CODIAK BIOSCIENCES, INC., (omitted)

(21) Appl. No.: 18/246,312

(22) PCT Filed: Sep. 23, 2021
(86) PCT No.: PCT/US2021/051783

§ 371 Date: Mar. 22, 2023

Related U.S. Application Data
(60) Provisional application No. 63/082,358, filed on Sep. 23, 2020.

(51) Int. Cl. G01N 1/34; B01D 15/36; B01D 15/38; B01D 15/42; B01D 15/20; C12N 9/22; C12N 15/10

(52) U.S. Cl. CPC G01N 1/34; B01D 15/363; B01D 15/362; B01D 15/3804; B01D 15/426; B01D 15/3847; B01D 15/203; C12Y 301/30002; C12N 9/22; C12N 15/101

(57) ABSTRACT

The present disclosure relates to multistep chromatographic methods for preparing extracellular vesicles (EVs). The methods were demonstrated to be effective in preparing high quality EVs in a large scale. The methods enable preparation of EVs for therapeutic and diagnostic applications, and isolation and/or sub-fractionation of EVs with desired properties for specific use.

### CROSS-REFERENCE TO EARLIER FILED APPLICATIONS

This PCT application claims the priority benefit of U.S. Provisional Application No. 63/082,358 filed on Sep. 23, 2020, which is incorporated by reference herein in its entirety.

### FIELD OF DISCLOSURE

The present disclosure provides multistep enzymatic and chromatographic methods for preparing extracellular vesicles (EVs). The methods are effective in preparing high-quality EVs, with low levels of contaminating nucleic acid molecules, on a large scale.

### BACKGROUND OF DISCLOSURE

Extracellular vesicles (EVs) are important mediators of intercellular communication. They are also important biomarkers in the diagnosis of many diseases, such as cancer. As drug delivery vehicles, EVs offer many advantages over traditional drug delivery methods, especially for gene therapy. The use of EVs for therapeutic purposes requires that EVs be free or mostly free of impurities including, but not limited to, undesirable nucleic acid molecules (e.g., DNA), host cell proteins, carbohydrates, and lipids. Current purification methods do not offer sufficient selectivity to remove significant amounts of these impurities so additional processes are desired to improve purity.

Furthermore, synthetic nano- and/or micro-carriers such as EVs often struggle to meet clinical expectations because of heterogeneity in their physicochemical parameters that confer targeting efficiency, immune evasion, and controlled drug release. This is mainly due to the complexity of nanoparticle properties (composition, size, shape, rigidity, surface charge, hydrophilicity, stability, and ligand type and density), payload properties (drug type, solubility, loading, potency, dosing, immune response, and release kinetics), and in vivo physiological barriers to nanoparticle trafficking (immune surveillance, particle extravasation, tissue targeting, tissue penetration, and cellular uptake). Although a considerable amount of effort has been made, effective methods for isolating discrete sub-populations of EVs (especially at scale) are not yet readily available.

In addition, therapeutic use of EVs requires larger-scale production and preparation of EVs. The heterogeneity and complexity of EVs make it difficult and costly to provide EVs in a large amount, while ensuring their quality. Inherent variability of the production and preparation process make it both expensive and unpredictable.

Therefore, effective and efficient methods for large-scale production, isolation and/or sub-fractionation of EVs are needed to enable use of EVs for therapeutic purposes.

### SUMMARY OF DISCLOSURE

Certain aspects of the present disclosure are directed to a method of preparing purified extracellular vesicles (EVs) from a sample comprising EVs and one or more nucleic acid molecules, comprising: (i) contacting the sample with a chromatography resin and (ii) contacting the chromatography resin with a nuclease wash buffer; wherein the nuclease wash buffer comprises a nuclease and a cation; and wherein the (ii) washing follows the (i) contacting.

Certain aspects of the present disclosure are directed to a method of reducing the concentration of residual nucleic acid molecule in a sample comprising extracellular vesicles (EVs), comprising (i) contacting the sample with a chromatography resin and (ii) contacting the chromatography resin with a nuclease wash buffer; wherein the nuclease wash buffer comprises a nuclease and a cation; and wherein the (ii) washing follows the (i) contacting.

Certain aspects of the present disclosure are directed to a method of preparing purified extracellular vesicles (EVs) from a sample comprising EVs and one or more nucleic acid molecules, comprising: (i) contacting the sample with a chromatography resin, (ii) contacting the chromatography resin with a wash buffer comprising a nuclease, a cation, or both a nuclease and a cation, (iii) eluting an eluent from the chromatography resin, wherein the eluent comprises the EVs, e.g., exosomes. In some aspects, the wash buffer comprises a nuclease. In some aspects, the wash buffer comprises a cation. In some aspects, the wash buffer comprises a nuclease and a cation. In some aspects, the method further comprises (iv) contacting the eluent with a nuclease.

In some aspects, the chromatography resin is selected from the group consisting of a cation exchange resin, an anion exchange (AEX) resin, an affinity chromatography resin, a pseudo affinity chromatography resin, a hydrophobic interaction resin, a hydrophobic charge induction chromatography resin, a mixed mode resin, an immobilized metal affinity resin, a ceramic hydroxyapatite resin, a fluoro hydroxyapatite resin, and any combination thereof. In some aspects, the chromatography resin comprises an AEX resin. In some aspects, the chromatography resin comprises a CEX resin. In some aspects, the chromatography resin comprises an affinity chromatography resin.

In some aspects, the nuclease is an endonuclease. In some aspects, the nuclease is an exonuclease. In some aspects, the nuclease is selected from salt active nuclease (SAN), benzonase, denarase, kryptonase, and any combination thereof. In some aspects, the nuclease comprises SAN.

In some aspects, the cation comprises a monovalent cation. In some aspects, the monovalent cation is selected from the group consisting of Li⁺, K⁺, Na⁺, NH₄⁺, Cu⁺, and any combination thereof. In some aspects, the cation comprises a divalent cation. In some aspects, the divalent cation is selected from the group consisting of Ca²⁺, Mg²⁺, Co²⁺, Ni²⁺, Zn²⁺, Ba²⁺, Sr²⁺, Al²⁺, Ag²⁺, Cu²⁺, Mn²⁺, and any combination thereof. In some aspects, the divalent cation comprises Mg²⁺. In some aspects, the cation is a buffer selected from the group consisting of an Imidazole, Tris, TAPS, BisTRIS, arginine, histidine, lysine buffer, and any combination thereof.

In some aspects, the nuclease wash buffer further comprises an anion. In some aspects, the anion is selected from SCN⁻, Cl⁻, SO₄⁻, PO₄⁻, Br⁻, I⁻, and any combination thereof. In some aspects, the anion is a buffer selected from the group consisting of a HEPES, BES, Bicine, MES, MOPS, PIPES, acetate, carbonate, citrate, bicarbonate buffer, aspartic acid, glutamic acid, and any combination thereof. In some aspects, the cation is associated with an anion, wherein the association is selected from MgCl₂, Mg(SCN)₂, MgSO₄)₂, Mg(PO₄)₂, and any combination thereof. In some aspects, the nuclease wash buffer comprises MgCl₂.

In some aspects, the sample is contacted with the chromatography resin in a loading buffer, wherein the loading buffer comprises a salt concentration from at least about 0.01 M to at least about 1.0 M. In some aspects, the salt concentration of the loading buffer is at least about 0.01 M, at least about 0.05 M, at least about 0.1 M, at least about 0.15 M, at least about 0.2 M, at least about 0.25 M, at least about 0.3 M, at least about 0.35 M, at least about 0.4 M, at least about 0.45 M, at least about 0.5 M, at least about 0.55 M, at least about 0.6 M, at least about 0.65 M, at least about 0.7 M, at least about 0.75 M, at least about 0.8 M, at least about 0.85 M, at least about 0.9 M, at least about 0.95 M, or at least about 1.0 M. In some aspects, the salt of the loading buffer is selected from NaCl, KCl, PO₄, CaCl₂), MgCl₂, and any combination thereof.

In some aspects, the salt of the loading buffer comprises NaCl. In some aspects, the loading buffer comprises at least about 0.01 M NaCl, at least about 0.05 M NaCl, at least about 0.1 M NaCl, at least about 0.15 M NaCl, at least about 0.2 M NaCl, at least about 0.25 M NaCl, at least about 0.3 M NaCl, at least about 0.35 M NaCl, at least about 0.4 M NaCl, at least about 0.45 M NaCl, at least about 0.5 M NaCl, at least about 0.55 M NaCl, at least about 0.6 M NaCl, at least about 0.65 M NaCl, at least about 0.7 M NaCl, at least about 0.75 M NaCl, at least about 0.8 M NaCl, at least about 0.85 M NaCl, at least about 0.9 M NaCl, at least about 0.95 M NaCl, or at least about 1 M NaCl. In some aspects, the loading buffer comprises at least about 0.55 M NaCl.

In some aspects, the nuclease wash buffer comprises at least about 1 unit/mL to at least about 100 units/mL, at least about 1 unit/mL to at least about 75 units/mL, at least about 1 unit/mL to at least about 50 units/mL, at least about 10 units/mL to at least about 100 units/mL, at least about 10 units/mL to at least about 75 units/mL, at least about 10 units/mL to at least about 50 units/mL, at least about 20 units/mL to at least about 100 units/mL, at least about 20 units/mL to at least about 75 units/mL, at least about 20 units/mL to at least about 50 units/mL, at least about 30 units/mL to at least about 100 units/mL, at least about 30 units/mL to at least about 75 units/mL, at least about 30 units/mL to at least about 50 units/mL, at least about 40 units/mL to at least about 100 units/mL, at least about 40 units/mL to at least about 75 units/mL, at least about 40 units/mL to at least about 50 units/mL, at least about 50 units/mL to at least about 100 units/mL, or at least about 50 units/mL to at least about 75 units/mL of the nuclease. In some aspects, the nuclease wash buffer comprises at least about 1 unit/mL, at least about 5 units/mL, at least about 10 units/mL, at least about 15 units/mL, at least about 20 units/mL, at least about 25 units/mL, at least about 30 units/mL, at least about 35 units/mL, at least about 40 units/mL, at least about 45 units/mL, at least about 50 units/mL, at least about 60 units/mL, at least about 65 units/mL, at least about 70 units/mL, at least about 80 units/mL, at least about 90 units/mL, or at least about 100 unit/mL of the nuclease.

In some aspects, the nuclease wash buffer comprises at least about 1 unit/mL to at least about 100 units/mL SAN. In some aspects, the nuclease wash buffer comprises at least about 1 unit/mL, at least about 5 units/mL, at least about 10 units/mL, at least about 15 units/mL, at least about 20 units/mL, at least about 25 units/mL, at least about 30 units/mL, at least about 35 units/mL, at least about 40 units/mL, at least about 45 units/mL, at least about 50 units/mL, at least about 60 units/mL, at least about 65 units/mL, at least about 70 units/mL, at least about 80 units/mL, at least about 90 units/mL, or at least about 100 unit/mL SAN. In some aspects, the nuclease wash buffer comprises at least about 40 units/mL SAN.

In some aspects, the nuclease wash buffer comprises at least about 0.01 M to at least about 1.0 M of the cation. In some aspects, the nuclease wash buffer comprises at least about 0.01 M, at least about 0.05 M, at least about 0.1 M, at least about 0.15 M, at least about 0.2 M, at least about 0.25 M, at least about 0.3 M, at least about 0.35 M, at least about 0.4 M, at least about 0.45 M, at least about 0.5 M, at least about 0.55 M, at least about 0.6 M, at least about 0.65 M, at least about 0.7 M, at least about 0.75 M, at least about 0.8 M, at least about 0.85 M, at least about 0.9 M, at least about 0.95 M, or at least about 1.0 M of the cation.

In some aspects, the cation comprises Mg²⁺, and wherein the concentration of the Mg²⁺ in the nuclease wash buffer is at least about 0.05 M, at least about 0.1 M, at least about 0.15 M, at least about 0.2 M, at least about 0.25 M, at least about 0.3 M, at least about 0.35 M, at least about 0.4 M, at least about 0.45 M, at least about 0.5 M, at least about 0.55 M, at least about 0.6 M, at least about 0.65 M, at least about 0.7 M, at least about 0.75 M, at least about 0.8 M, at least about 0.85 M, at least about 0.9 M, at least about 0.95 M, or at least about 1.0 M Mg²⁺. In some aspects, the concentration of the Mg²⁺ in the nuclease wash buffer is at least about 0.35 M Mg²⁺. In some aspects, the nuclease wash buffer comprises at least about 0.35 M MgCl₂.

In some aspects, the nuclease wash buffer is contacted with the chromatography resin at least 2 times, at least 3 times, at least 4 times, or at least 5 times.

In some aspects, the method further comprises washing the chromatography resin by contacting the chromatography resin with a wash buffer, wherein the wash buffer does not comprise a nuclease. In some aspects, the chromatography resin is contacted with the wash buffer (a) after (i) contacting the sample with a chromatography resin, and before (ii) contacting the chromatography resin with a nuclease wash buffer (b) after (ii) contacting the chromatography resin with a nuclease wash buffer (c) both (a) and (b).

In some aspects, the wash buffer comprises a salt at a concentration of at least about 0.01 M, at least about 0.05 M, at least about 0.1 M, at least about 0.15 M, at least about 0.2 M, at least about 0.25 M, at least about 0.3 M, at least about 0.35 M, at least about 0.4 M, at least about 0.45 M, at least about 0.5 M, at least about 0.55 M, at least about 0.6 M, at least about 0.65 M, at least about 0.7 M, at least about 0.75 M, at least about 0.8 M, at least about 0.85 M, at least about 0.9 M, at least about 0.95 M, or at least about 1.0 M. In some aspects, the salt in the wash buffer is selected from NaCl, KCl, PO₄, CaCl₂), MgCl₂, and any combination thereof. In some aspects, the salt in the wash buffer is selected from NaCl, KCl, KPO₄, NaPO₄, CaCl₂), Mg₂SO₄, ZnCl₂, MnCl₂, MnSO₄, NaSCN, KSCN, LiCl, MgCl₂, and any combination thereof.

In some aspects, the wash buffer comprises NaCl. In some aspects, the wash buffer comprises at least about 0.01 M NaCl, at least about 0.05 M NaCl, at least about 0.1 M NaCl, at least about 0.15 M NaCl, at least about 0.2 M NaCl, at least about 0.25 M NaCl, at least about 0.3 M NaCl, at least about 0.35 M NaCl, at least about 0.4 M NaCl, at least about 0.45 M NaCl, at least about 0.5 M NaCl, at least about 0.55 M NaCl, at least about 0.6 M NaCl, at least about 0.65 M NaCl, at least about 0.7 M NaCl, at least about 0.75 M NaCl, at least about 0.8 M NaCl, at least about 0.85 M NaCl, at least about 0.9 M NaCl, at least about 0.95 M NaCl, or at least about 1 M NaCl. In some aspects, the wash buffer comprises at least about 1.1 M NaCl, at least about 1.2 M NaCl, at least about 1.3 M NaCl, at least about 1.4 M NaCl, at least about 1.5 M NaCl, at least about 1.6 M NaCl, at least about 1.7 M NaCl, at least about 1.8 M NaCl, at least about 1.9 M NaCl, at least about 2 M NaCl. In some aspects, the wash buffer comprises at least about 0.55 M NaCl.

In some aspects, the method further comprises (iii) eluting the EVs from the chromatography resin by contacting the chromatography resin with an elution buffer, wherein (iii) occurs after (ii) contacting the chromatography resin with a nuclease wash buffer. In some aspects, the elution buffer comprises a salt concentration of at least about 1.0 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2.0 M, at least about 2.5 M, at least about 3.0 M, at least about 3.5 M, at least about 4.0 M, at least about 4.5 M, or at least about 5.0 M. In some aspects, the elution buffer comprises a salt concentration of at least about 1.0 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2.0 M, at least about 2.5 M, at least about 3.0 M, at least about 3.5 M, at least about 4.0 M, at least about 4.5 M, or at least about 5.0 M NaCl. In some aspects, the elution buffer comprises at least about 1.2 M NaCl.

In some aspects, the elution buffer releases one or more EVs from the chromatography resin. In some aspects, the method further comprises collecting an eluent after contacting the chromatography resin with the elution buffer. In some aspects, the eluent comprises one or more EVs.

In some aspects, the sample contacted with the chromatography resin comprises a starting concentration of the one or more nucleic acid molecules, and wherein the eluent comprises an eluted concentration of the one or more nucleic acid molecules, wherein the eluted concentration of the one or more nucleic acid molecules is less than about 10%, less than about 5%, less than about 1%, less than about 0.5%, less than about 0.1%, less than about 0.05%, less than about 0.01%, less than about 0.001%, or less than about 0.0001% that of the starting concentration of the one or more nucleic acid molecules.

In some aspects, the method further comprises subjecting the sample to one or more additional chromatography resins. In some aspects, the one or more additional chromatography resins comprises an anion exchange chromatography (AEX) resin, a cation exchange chromatography (CEX) resin, a mixed mode chromatography (MMC) resin, hydrophobic charge induction chromatography resin, a hydrophobic interaction chromatography resin, or any combination thereof.

In some aspects, the sample is contacted with a CEX resin after the AEX resin. In some aspects, the sample is contacted with an MMC resin after the CEX resin. In some aspects, the sample is contacted with an MMC resin after the AEX resin. In some aspects, the sample is contacted with a CEX resin, an affinity resin, an IC, a ceramic hydroxyapatite, a CFT, an IMAC, or any combination thereof after the MMC resin.

In some aspects, the sample is contacted with the chromatography resin and/or the additional chromatography resin at least two times, at least three times, at least four times, at least five times, at least six times, at least seven times, at least eight times, at least nine times, at least eight times, at least nine times, at least ten times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, or at least 25 times.

In some aspects, the sample is contacted with (a) an AEX resin (b) an CEX resin (c) an MMC resin (d) an affinity chromatography resin (e) an HIC resin (f) a ceramic hydroxyapatite resin (g) an IMAC resin (h) an HCIC resin (i) any combination thereof.

In some aspects, the EV is an exosome.

Some aspects of the present disclosure are directed to a method of preparing purified extracellular vesicles (EVs) from a sample comprising EVs and one or more nucleic acid molecules, comprising: (i) contacting the sample with a chromatography resin and (ii) contacting the chromatography resin with a wash buffer comprising a nuclease, a cation, or both a nuclease and a cation; and wherein the (ii) washing follows the (i) contacting.

Some aspects of the present disclosure are directed to a method of reducing the concentration of residual nucleic acid molecule in a sample comprising extracellular vesicles (EVs), comprising (i) contacting the sample with a chromatography resin and (ii) contacting the chromatography resin with a wash buffer comprising a nuclease, a cation, or both a nuclease and a cation; and wherein the (ii) washing follows the (i) contacting.

Some aspects of the present disclosure are directed to a method of preparing purified extracellular vesicles (EVs) from a sample comprising EVs and one or more nucleic acid molecules, comprising: (i) contacting the sample with a chromatography resin; (ii) contacting the chromatography resin with a wash buffer comprising a nuclease, a cation, or both a nuclease and a cation; and (iii) eluting an eluent from the chromatography resin, wherein the eluent comprises the EVs, e.g., exosomes.

In some aspects, the wash buffer comprises a nuclease (“a nuclease wash buffer”). In some aspects, the wash buffer comprises a cation. In some aspects, the method further comprises (iv) contacting the eluent with a nuclease wash buffer.

Some aspects of the present disclosure are directed to a method of preparing purified extracellular vesicles (EVs) from a sample comprising EVs and one or more nucleic acid molecules, comprising: (i) contacting the sample with a chromatography resin, (ii) eluting an eluent form the chromatography resin, wherein the eluent comprises the EVs, e.g., exosomes, and (iii) contacting the eluent with a nuclease wash buffer.

Some aspects of the present disclosure are directed to a method of reducing the concentration of residual nucleic acid molecule in a sample comprising extracellular vesicles (EVs), comprising (i) contacting the sample with a chromatography resin, (ii) eluting an eluent form the chromatography resin, wherein the eluent comprises the EVs, e.g., exosomes, and (iii) contacting the eluent with a nuclease wash buffer. In some aspects, the nuclease wash buffer comprises a nuclease and a cation.

In some aspects, the chromatography resin is selected from the group consisting of a cation exchange resin, an anion exchange (AEX) resin, an affinity chromatography resin, a pseudo affinity chromatography resin, a hydrophobic interaction resin, a hydrophobic charge induction chromatography resin, a mixed mode resin, an immobilized metal affinity resin, a ceramic hydroxyapatite resin, a fluoro hydroxyapatite resin, and any combination thereof. In some aspects, the chromatography resin comprises an AEX resin. In some aspects, the chromatography resin comprises a CEX resin. In some aspects, the chromatography resin comprises an affinity chromatography resin.

In some aspects, the nuclease is an endonuclease. In some aspects, the nuclease is an exonuclease. In some aspects, the nuclease is selected from salt active nuclease (SAN), benzonase, denarase, kryptonase, and any combination thereof. In some aspects, the nuclease comprises SAN.

In some aspects, the cation comprises a monovalent cation. In some aspects, the monovalent cation is selected from the group consisting of Li⁺, K⁺, Na⁺, NH4+, Cu⁺, and any combination thereof. In some aspects, the cation comprises a divalent cation. In some aspects, the divalent cation is selected from the group consisting of Ca²⁺, Mg²⁺, Co²⁺, Ni²⁺, Zn²⁺, Ba²⁺, Sr²⁺, Al²⁺, Ag²⁺, Cu²⁺, Mn²⁺, and any combination thereof. In some aspects, the divalent cation comprises Mg²⁺. In some aspects, the cation is a buffer selected from the group consisting of an Imidazole, Tris, TAPS, BisTRIS, arginine, histidine, lysine buffer, and any combination thereof.

In some aspects, the nuclease wash buffer further comprises an anion. In some aspects, the anion is selected from SCN, Cl⁻, SO₄⁻, PO₄⁻, Br⁻, I⁻, and any combination thereof. In some aspects, the anion is a buffer selected from the group consisting of a HEPES, BES, Bicine, MES, MOPS, PIPES, acetate, carbonate, citrate, bicarbonate buffer, aspartic acid, glutamic acid, and any combination thereof. In some aspects, the cation is associated with an anion, wherein the association is selected from MgCl₂, Mg(SCN)₂, Mg(SO₄)₂, Mg(PO₄)₂, and any combination thereof. In some aspects, the nuclease wash buffer comprises MgCl₂.

In some aspects, the sample is contacted with the chromatography resin in a loading buffer, wherein the loading buffer comprises a salt concentration from at least about 0.01 M to at least about 2.0 M. In some aspects, the salt concentration of the loading buffer is at least about 0.01 M, at least about 0.05 M, at least about 0.1 M, at least about 0.15 M, at least about 0.2 M, at least about 0.25 M, at least about 0.3 M, at least about 0.35 M, at least about 0.4 M, at least about 0.45 M, at least about 0.5 M, at least about 0.55 M, at least about 0.6 M, at least about 0.65 M, at least about 0.7 M, at least about 0.75 M, at least about 0.8 M, at least about 0.85 M, at least about 0.9 M, at least about 0.95 M, or at least about 1.0 M.

In some aspects, the salt of the loading buffer is selected from NaCl, KCl, KPO₄, NaPO₄, CaCl₂), Mg₂SO₄, ZnCl₂, MnCl₂, MnSO₄, NaSCN, KSCN, LiCl, MgCl₂, and any combination thereof. In some aspects, the salt of the loading buffer comprises NaCl. In some aspects, the loading buffer comprises at least about 0.01 M NaCl, at least about 0.05 M NaCl, at least about 0.1 M NaCl, at least about 0.15 M NaCl, at least about 0.2 M NaCl, at least about 0.25 M NaCl, at least about 0.3 M NaCl, at least about 0.35 M NaCl, at least about 0.4 M NaCl, at least about 0.45 M NaCl, at least about 0.5 M NaCl, at least about 0.55 M NaCl, at least about 0.6 M NaCl, at least about 0.65 M NaCl, at least about 0.7 M NaCl, at least about 0.75 M NaCl, at least about 0.8 M NaCl, at least about 0.85 M NaCl, at least about 0.9 M NaCl, at least about 0.95 M NaCl, at least about 1 M NaCl, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M or at least about 2 M. In some aspects, the loading buffer comprises at least about 0.55 M NaCl.

In some aspects, the nuclease wash buffer comprises at least about 0.001 unit/mL to at least about 1000 units/mL, at least about 1 unit/mL to at least about 100 units/mL, at least about 1 unit/mL to at least about 75 units/mL, at least about 1 unit/mL to at least about 50 units/mL, at least about 10 units/mL to at least about 100 units/mL, at least about 10 units/mL to at least about 75 units/mL, at least about 10 units/mL to at least about 50 units/mL, at least about 20 units/mL to at least about 100 units/mL, at least about 20 units/mL to at least about 75 units/mL, at least about 20 units/mL to at least about 50 units/mL, at least about 30 units/mL to at least about 100 units/mL, at least about 30 units/mL to at least about 75 units/mL, at least about 30 units/mL to at least about 50 units/mL, at least about 40 units/mL to at least about 100 units/mL, at least about 40 units/mL to at least about 75 units/mL, at least about 40 units/mL to at least about 50 units/mL, at least about 50 units/mL to at least about 100 units/mL, or at least about 50 units/mL to at least about 75 units/mL of the nuclease. In some aspects, the nuclease wash buffer comprises at least about 1 unit/mL, at least about 5 units/mL, at least about 10 units/mL, at least about 15 units/mL, at least about 20 units/mL, at least about 25 units/mL, at least about 30 units/mL, at least about 35 units/mL, at least about 40 units/mL, at least about 45 units/mL, at least about 50 units/mL, at least about 60 units/mL, at least about 65 units/mL, at least about 70 units/mL, at least about 80 units/mL, at least about 90 units/mL, or at least about 100 unit/mL of the nuclease.

In some aspects, the nuclease wash buffer comprises at least about 1 unit/mL to at least about 100 units/mL SAN. In some aspects, the nuclease wash buffer comprises at least about 1 unit/mL, at least about 5 units/mL, at least about 10 units/mL, at least about 15 units/mL, at least about 20 units/mL, at least about 25 units/mL, at least about 30 units/mL, at least about 35 units/mL, at least about 40 units/mL, at least about 45 units/mL, at least about 50 units/mL, at least about 60 units/mL, at least about 65 units/mL, at least about 70 units/mL, at least about 80 units/mL, at least about 90 units/mL, or at least about 100 unit/mL SAN. In some aspects, the nuclease wash buffer comprises at least about 40 units/mL SAN.

In some aspects, the nuclease wash buffer comprises at least about 0.01 M to at least about 1.0 M of the cation. In some aspects, the nuclease wash buffer comprises at least about 0.01 M, at least about 0.05 M, at least about 0.1 M, at least about 0.15 M, at least about 0.2 M, at least about 0.25 M, at least about 0.3 M, at least about 0.35 M, at least about 0.4 M, at least about 0.45 M, at least about 0.5 M, at least about 0.55 M, at least about 0.6 M, at least about 0.65 M, at least about 0.7 M, at least about 0.75 M, at least about 0.8 M, at least about 0.85 M, at least about 0.9 M, at least about 0.95 M, or at least about 1.0 M of the cation.

In some aspects, the cation comprises Mg²⁺, and wherein the concentration of the Mg²⁺ in the nuclease wash buffer is at least about 0.05 M, at least about 0.1 M, at least about 0.15 M, at least about 0.2 M, at least about 0.25 M, at least about 0.3 M, at least about 0.35 M, at least about 0.4 M, at least about 0.45 M, at least about 0.5 M, at least about 0.55 M, at least about 0.6 M, at least about 0.65 M, at least about 0.7 M, at least about 0.75 M, at least about 0.8 M, at least about 0.85 M, at least about 0.9 M, at least about 0.95 M, or at least about 1.0 M Mg²⁺. In some aspects, the concentration of the Mg²⁺ in the nuclease wash buffer is at least about 0.35 M Mg²⁺. In some aspects, the nuclease wash buffer comprises at least about 0.35 M MgCl₂.

In some aspects, the nuclease wash buffer is contacted with the chromatography resin at least 2 times, at least 3 times, at least 4 times, or at least 5 times.

In some aspects, the eluent is contacted with the nuclease wash buffer by incubation for at least about 1 minute, at least about 2 minutes, at least about 3 minutes, at least about 4 minutes, at least about 5 minutes, at least about 10 minutes, at least about 15 minutes, at least about 20 minutes, at least about 25 minutes, at least about 30 minutes, at least about 60 minutes, at least about 90 minutes, at least about 120 minutes, at least about 180 minutes, at least about 3 hours, at least about 4 hours, at least about 5 hours, at least about 6 hours, at least about 8 hours, at least about 9 hours, at least about 10 hours, at least about 11 hours, at least about 12 hours, at least about 15 hours, at least about 18 hours, at least about 21 hours, or at least about 24 hours. In some aspects, the eluent is contacted with the nuclease wash buffer by incubation for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days.

In some aspects, the eluent is contacted with the nuclease wash buffer by incubation at about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., or about 37° C. In some aspects, the eluent is contacted with the nuclease wash buffer by incubation at about 4° C. In some aspects, the eluent is contacted with the nuclease wash buffer by incubation at about 37° C.

In some aspects, the method further comprises (iii) eluting the EVs from the chromatography resin, wherein (iii) occurs after (ii) contacting the chromatography resin with the wash buffer.

In some aspects, the EVs are eluted from the chromatography resin by contacting the chromatography resin with an elution buffer. In some aspects, the elution buffer comprises a salt concentration of at least about 1.0 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2.0 M, at least about 2.5 M, at least about 3.0 M, at least about 3.5 M, at least about 4.0 M, at least about 4.5 M, or at least about 5.0 M. In some aspects, the elution buffer comprises a salt concentration of at least about 1.0 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2.0 M, at least about 2.5 M, at least about 3.0 M, at least about 3.5 M, at least about 4.0 M, at least about 4.5 M, or at least about 5.0 M NaCl. In some aspects, the elution buffer comprises at least about 1.2 M NaCl. In some aspects, the elution buffer releases one or more EVs from the chromatography resin.

In some aspects, the sample contacted with the chromatography resin comprises a starting concentration of the one or more nucleic acid molecules, and wherein the eluent comprises an eluted concentration of the one or more nucleic acid molecules, wherein the eluted concentration of the one or more nucleic acid molecules is less than about 10%, less than about 5%, less than about 1%, less than about 0.5%, less than about 0.1%, less than about 0.05%, less than about 0.01%, less than about 0.001%, or less than about 0.0001% that of the starting concentration of the one or more nucleic acid molecules.

In some aspects, the method further comprises subjecting the sample to one or more additional chromatography resins. In some aspects, the one or more additional chromatography resins comprises an anion exchange chromatography (AEX) resin, a cation exchange chromatography (CEX) resin, a mixed mode chromatography (MMC) resin, hydrophobic charge induction chromatography resin, a hydrophobic interaction chromatography resin, an immobilized metal affinity chromatography (IMAC), or any combination thereof. In some aspects, the sample is contacted with a CEX resin after the AEX resin. In some aspects, the sample is contacted with an MMC resin after the CEX resin. In some aspects, the sample is contacted with an MMC resin after the AEX resin. In some aspects, the sample is contacted with a CEX resin, an affinity resin, an HIC, a ceramic hydroxyapatite, a CFT, an IMAC, or any combination thereof after the MMC resin. In some aspects, the sample is contacted with the chromatography resin and/or the additional chromatography resin at least two times, at least three times, at least four times, at least five times, at least six times, at least seven times, at least eight times, at least nine times, at least eight times, at least nine times, at least ten times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, or at least 25 times. In some aspects, the sample is contacted with, an AEX resin, an CEX resin, an MMC resin, an affinity chromatography resin, an HIC resin, a ceramic hydroxyapatite resin, an IMAC resin, an HCIC resin, or any combination thereof.

In some aspects, the EV is an exosome.

In some aspects, the concentration of EVs in the sample is at least about 1E9 to about 1E14 p/mL.

Certain aspects of the present disclosure are directed to a composition comprising extracellular vesicles prepared by any method disclosed herein. In some aspects, the composition further comprises (a) a saccharide (b) sodium chloride (c) a potassium phosphate (d) a sodium phosphate (e) any combination thereof.

Certain aspects of the present disclosure are directed to a method of treating a disease or condition in a subject in need thereof comprising administering any composition disclosed herein.

### BRIEF DESCRIPTION OF THE FIGURES

FIG. 1 is a schematic representation of control and experimental DNA clearance processes comprising use of anion exchange chromatography (AEX) with or without a nuclease wash or a nuclease incubation.

FIG. 2 is a schematic representation of an experimental design used for an AEX chromatography wash buffer assessment. The concentration of MgCl₂ (M), concentration of salt active nuclease (SAN) (U/mL), and whether or not the load condition comprised 20 U/mL of Benzonase (X represents 0 U/mL Benzonase in the load; + represents 20 U/mL Benzonase in the load) are depicted by the schematic representation.

FIGS. 3A-3B are graphical representations of the residual DNA concentration as ng/mL (FIG. 3A) or normalized as ng/1E11 p (FIG. 3B), as measured by qPCR, of various different AEX eluate samples as compared to the AEX wash buffer used during AEX for a given eluate sample. For FIG. 3A, the X-axis represents the wash buffer conditions used, and the Y-axis represents the residual DNA concentration in ng/mL as measured by qPCR. The closed circle data markers indicate that no AEX load nuclease treatment was used for a given sample, and the closed triangle data markers indicate that a 20 U/mL Benzonase AEX load nuclease treatment was used for a given sample. For FIG. 3B, the Y-axis represents ng DNA per 1E11 particles as measured by qPCR. The closed circle data markers indicate that no AEX load nuclease treatment was used for a given sample, and the closed square data marker indicates that a 20 U/mL Benzonase AEX load nuclease treatment was used.

FIG. 4A is a graphical representation of the residual DNA concentration, as measured by the A254/A280 peak area ratio, of various different AEX eluate samples as compared to the AEX wash buffer used during AEX for a given eluate sample. The X-axis represents the wash buffer conditions used, and the Y-axis represents the A254/A280 peak area ratio of the various different AEX eluate samples. The solid bars indicate that no AEX load nuclease treatment was used for a given sample, and the hatched bars indicate that a 20 U/mL Benzonase AEX load nuclease treatment was used for a given sample. FIG. 4B shows a full chromatogram of clarified harvest material containing exosomes purified through an anion exchange membrane chromatography process. Column flow through signals are visualized using UV254 nm, UV280 nm and conductivity. Chromatography is operated in bind and elute mode, and impurities and product are selectively desorbed from the membrane using isocratic elution with either magnesium chloride or sodium chloride. The MgCl₂ DNA removal wash is shown in the black box, and further in FIG. 4C. The peak desorbed with MgCl₂ in FIG. 4C has an A254/A280 nm ratio of 1.61, which further supports that it is enriched in nucleic acid content. FIG. 4D is a bar graph showing the LRV clearance following either MgCl₂ wash, SAN digestion, or both, as indicated.

FIG. 5 is a graphical representation of the particle counts of various different AEX eluate samples as compared to the AEX wash buffer used during AEX. The X-axis represents the wash buffer conditions used, and the Y-axis represents the particle counts (p/mL) as measured by nanoparticle tracking analysis (NTA) of the various different AEX eluate samples. The solid bars indicate that no AEX load nuclease treatment was used for a given sample, and the hatched bars indicate that a 20 U/mL Benzonase AEX load nuclease treatment was used for a given sample.

FIG. 6 is a graphical representation of the protein content as indicated by BCA assay of various different AEX eluate samples as compared to the AEX wash buffer used during AEX. The X-axis represents the wash buffer conditions used, and the Y-axis represents the protein content (μg/mL) of the various different AEX eluate samples as measured by BCA assay. The solid bars indicate that no AEX load nuclease treatment was used for a given sample, and the hatched bars indicate that a 20 U/mL Benzonase AEX load nuclease treatment was used for a given sample.

FIGS. 7A-7B are graphical representations of the polydispersity index (PDI) as measured by dynamic light scattering (DLS) (FIG. 7A) and particle diameters as measured by DLS (FIG. 7B) of various different AEX eluate samples as compared to the AEX wash buffer used during AEX. The X-axes represent the wash buffer conditions used; the Y-axes represent the polydispersity index (PDI) as measured by DLS (FIG. 7A) and the particle diameter measurements (nm) as measured by DLS (FIG. 7B) of the various different AEX eluate samples. The solid bars indicate that no AEX load nuclease treatment was used for a given sample, and the hatched bars indicate that a 20 U/mL Benzonase AEX load nuclease treatment was used for a given sample (FIGS. 7A-7B).

FIG. 8 is a table illustrating an experimental design for comparing various different AEX eluate nuclease treatment conditions (both test and control conditions).

FIGS. 9A-9B are graphical representations of the polydispersity index (PDI) as measured by dynamic light scattering (DLS) (FIG. 9A) and particle diameters as measured by DLS (FIG. 9B) of various different AEX eluate samples as compared to the final enzyme (nuclease) concentration of each of the AEX eluate samples. The X-axis represents the final concentration of enzyme (nuclease) (U/mL) in a given sample; the Y-axes represent the polydispersity index (PDI) as measured by dynamic light scattering (DLS; FIG. 9A) and diameter measurements as measured by DLS (FIG. 9B) of a given sample.

FIG. 10A is a graphical representation of the cholesterol content of various different AEX eluate samples as compared to the final enzyme (nuclease) concentration of each of the AEX eluate samples. The X-axis represents the final concentration of enzyme (nuclease) (U/mL) in a given sample; the Y-axis represents the concentration of cholesterol (μg/mL) of a given sample. FIG. 10B is a graphical representation of residual DNA amount of various different AEX eluate samples as compared to the final enzyme (nuclease) concentration of each of the AEX eluate samples. The X-axis represents the final concentration of enzyme (nuclease) (U/mL) in a given sample; the Y-axis represents the concentration of residual DNA (ng/mL) as measured by qPCR of a given sample.

FIG. 11 is a graphical representation of the ratio of residual DNA amount (ng) to the amount of cholesterol (μg) of various different AEX eluate samples as compared to the final enzyme (nuclease) concentration of each of the AEX eluate samples. The X-axis represents the final concentration of enzyme (nuclease) (U/mL) in a given sample; the Y-axis represents the ratio of residual DNA amount (ng) as measured by qPCR to the amount of cholesterol (μg) of said AEX eluate samples.

FIG. 12A is a schematic representation of an experimental design used to evaluate AEX eluate nuclease treatment with SAN. FIG. 12B shows the residual DNA content (ng/1E11 p), as measured by qPCR of various AEX eluate samples treated with SAN. The X axes describe SAN concentration and hold time and the Y axis describes represents ng DNA per 1E11 particles as measured by qPCR.

FIG. 13 is a schematic of an exemplary large-scale purification process of extracellular vesicles (EVs) for the removal of nucleic acid impurity. This process comprises a filter-based clarification step prior to the anion exchange chromatography (AEX) step. As described herein, the process can further comprise one or more additional chromatography steps (e.g., mixed mode chromatography) after the AEX step, as shown.

FIG. 14 is a bar graph delineating DNA removal across an exosome purification process that uses salt active nuclease to digest residual nucleic acids (see FIG. 13). Greater than three logs of DNA removal were observed in two separate lots of the purified final pool.

FIG. 15 is a bar graph showing DNA removal as a function of 0.35M MgCl₂ residence time. The wash residence time is shown on the X-axis and the residual content of the pre- and post-SAN treated AEX eluates are shown on the Y-axis.

FIG. 16 is a bar graph showing the effect of MgCl₂ concentration (X-axis) on the residual DNA content of SAN-treated AEX eluates.

FIG. 17 is a graphical representation showing consistency of DNA clearance across AEX membrane scales and eluate pool volumes.

FIG. 18 is a scatter plot showing the effect of hold time (X-axis) and temperature on DNA digestion of untreated AEX eluate material. The digestion of DNA occurs quickly and reaches a stable consistent value whether in ambient (15-25° C.; circles) or cold (2-8° C.; triangles) hold conditions.

### DETAILED DESCRIPTION OF DISCLOSURE

The present disclosure provides purification processes of extracellular vesicles (EVs), utilizing multiple steps of chromatography. The EVs processed by the present methods can be highly purified, e.g., less nucleic acid molecule impurities, higher potency, higher uniformity, or any combination thereof.

Certain aspects of the present disclosure are directed to methods of preparing purified extracellular vesicles (EVs) from a sample comprising EVs and one or more nucleic acid molecules, comprising: (i) contacting the sample with a chromatography resin and (ii) contacting the chromatography resin with a nuclease wash buffer, wherein the (ii) washing follows the (i) contacting. Some aspects of the present disclosure are directed to methods of reducing the concentration of residual nucleic acid molecule in a sample comprising extracellular vesicles (EVs), comprising (i) contacting the sample with a chromatography resin and (ii) contacting the chromatography resin with a nuclease wash buffer, wherein the (ii) washing follows the (i) contacting. In some aspects, the nuclease wash buffer comprises a nuclease and a cation. In some aspects, nuclease comprises salt active nuclease (SAN). In some aspects, the cation comprises a divalent cation. In certain aspects, the cation comprises Mga+.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values, which are about the same quantity or amount as the recited value are also within the scope of the disclosure. Where a combination is disclosed, each sub-combination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, and U represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “large scale” refers to a production scale that is larger than an experimental or laboratory use for research purposes only. Large scale purification is the final production step, prior to product formulation, in the manufacture of therapeutic products, e.g., EVs. Large-scale purification requires a scale-up from laboratory scale techniques to satisfy the need for larger amounts of extremely pure test quantities of the product for analysis, characterization, testing of efficacy, clinical or field trials, and, finally, full scale commercialization. The uncompromising standards for product quality, as well as rigorous quality control of manufacturing practices embodied in current good manufacturing practices (cGMP's), provide further challenges to the scale-up of EV purification. Analysis of electrokinetic, chromatographic, adsorptive, and membrane separation techniques suggests that if yield recovery is paramount, documented purity is critical, and both must ultimately be attained within certain cost constraints. The term “large scale” as used herein indicates that the final product is for use in clinical settings and commercial sales of the purified EV products. In some aspects, the term “large scale” purification means a purification process of at least about 500 L, at least about 550 L, at least about 600 L, at least about 650 L, at least about 700 L, at least about 750 L, at least about 800 L, at least about 850 L, at least about 900 L, at least about 950 L, at least about 1000 L, at least about 1500 L, or at least about 2000 L cell culture harvest. In some aspects, the term “large scale” purification means a purification process of at least about 2000 L cell culture harvest. In some aspects, the term “large scale” purification means a purification process of at least about 3000 L, at least about 4000 L, at least about 5000 L, at least about 6000 L, at least about 7000 L, at least about 8000 L, at least about 9000 L, at least about 10,000 L, at least about 11,000 L, at least about 12000 L, at least about 13,000 L, at least about 14,000 L, or at least about 15,000 L cell culture harvest.

As used herein, the terms chromatography “resin” and “matrix” are used interchangeably, and refer to the stationary (e.g., solid) phase of a chromatography (e.g., a column chromatography or a membrane chromatography). The methods disclosed herein can be applied to any form of chromatography suitable for the purification of EVs, e.g., exosomes. In some aspects, the chromatography resin is present in a packed column (e.g., a packed-bed column chromatography). In some aspects, the chromatography resin is present on a convective media (e.g., a membrane (e.g., a membrane chromatography), one or more monoliths, one or more fibers, or any combination thereof). In certain aspects, the chromatography resin comprises an “affinity” chromatography resin, which refers to a chromatography resin that interacts with one or more molecules present in the mobile phase of the chromatography. An affinity chromatography can be used in a “bind-and-elute” mode, wherein the desired molecules interact with the stationary phase until certain conditions are created that cause the desired molecules to release from the stationary phase and elute from the chromatography resin; or in a “pass through” mode, wherein one or more impurities present in the mobile phase, but not the desired molecules, interact with the chromatography resin, allowing the desired molecules to “pass through” the chromatography resin, while the impurities remain associated with the chromatography resin. In some aspects, the chromatography resin comprises an anion exchange (AEX) resin, a cation exchange (CEX) resin, a pseudo affinity chromatography resin, a hydrophobic interaction resin, a hydrophobic charge induction chromatography resin, a mixed mode resin, an immobilized metal affinity resin, a ceramic hydroxyapatite resin, a fluoro hydroxyapatite resin, and any combination thereof. In some aspects, the chromatography resin comprises a mixed-mode chromatography (MMC) resin.

As used herein, the term “extracellular vesicle” or “EV” refers to a cell-derived vesicle comprising a membrane that encloses an internal space. Extracellular vesicles comprise all membrane-bound vesicles (e.g., exosomes, microvesicles, microsomes, extracellular bodies, apoptotic bodies, and/or nanovesicles) that have a smaller diameter than the cell from which they are derived. In some aspects, extracellular vesicles comprise a population of exosomes and/or microvesicles. In some aspects, extracellular vesicles range in diameter from 20 nm to 1000 nm, and can comprise various macromolecular molecules either within the internal space (i.e., lumen), displayed on the external surface and/or the luminal surface of the EV, and/or spanning the membrane. In some aspects, the molecules in the EVs can comprise nucleic acids, proteins, carbohydrates, lipids, small molecules, and/or combinations thereof. In certain aspects, an EV comprises a scaffold moiety. By way of example and without limitation, EVs include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). EVs can be derived from a living or dead organism, explanted tissues or organs, prokaryotic or eukaryotic cells, and/or cultured cells. In some aspects, the EVs are produced by cells that express one or more transgene products. The EVs that can be purified by the present methods include exosomes, microsomes, microvesicles, extracellular bodies, apoptotic bodies, nanovesicles, or any combination thereof.

As used herein, the term “exosome” refers to an extracellular vesicle with a diameter between 20-300 nm (e.g., between 40-200 nm). Exosomes comprise a membrane that encloses an internal space (i.e., lumen), and, in some aspects, can be generated from a cell (e.g., producer cell) by direct plasma membrane budding or by fusion of the late endosome with the plasma membrane. As described infra, an exosome can be derived from a producer cell, and isolated from the producer cell based on its size, density, biochemical parameters, or a combination thereof.

In some aspects, the exosome of the present disclosure is engineered by associating (e.g., linking, e.g., covalently linking) at least one moiety, e.g., payload, e.g., a biologically active molecule (e.g., a protein such as an antibody or ADC, a RNA or DNA such as an antisense oligonucleotide, a small molecule drug, a toxin, a STING agonist, or PROTAC) to the exosome, directly or indirectly, e.g., via a linker, a scaffold moiety, or any combination thereof.

As used herein, the term “payload” refers to an agent that acts on a target (e.g., a target cell) that is contacted with the EV (e.g., exosome). In some aspects, unless indicated otherwise, the term payload can be used interchangeably with the term “biologically active molecule.” Non-limiting examples of payload that can be included on the EV, e.g., exosome, are polypeptides (e.g., an antibody, an antigen, an adjuvant, a ligand, a receptor, an immune modulator, and or any fragment thereof), a polynucleotide, a viral particle, a small molecule, or any combination thereof. Payloads that can be introduced into an EV, e.g., exosome, and/or a producer cell include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, siRNA, antisense oligonucleotide, a phosphorodiamidate morpholino oligomer (PMO), a peptide-conjugated phosphorodiamidate morpholino oligomer (PPMO), or combinations thereof), amino acids (e.g., amino acids comprising a detectable moiety or a toxin or that disrupt translation), polypeptides (e.g., enzymes), lipids, carbohydrates, and small molecules (e.g., small molecule drugs and toxins). In certain aspects, a payload comprises an antigen.

In some aspects, the payload is a protein, a peptide, a glycolipid, or a glycoprotein.

In certain aspects, the payload is a polynucleotide. In some of these aspects, the polynucleotide includes, but is not limited to, an mRNA, a miRNA, an siRNA, an antisense oligonucleotide (e.g., antisense RNA or antisense DNA), a phosphorodiamidate morpholino oligomer (PMO), a peptide-conjugated phosphorodiamidate morpholino oligomer (PPMO), an shRNA, a lncRNA, a dsDNA, and combinations thereof. In some aspects, the polynucleotide is an RNA (e.g., an mRNA, a miRNA, an siRNA, an antisense oligonucleotide (e.g., antisense RNA), an shRNA, or an lncRNA). In some aspects, the polynucleotide can target a transcription factor. In some of these aspects, when the polynucleotide is an mRNA, it can be translated into a desired polypeptide. In some aspects, the polynucleotide is a microRNA (miRNA) or pre-miRNA molecule. In some of these aspects, the miRNA is delivered to the cytoplasm of the target cell, such that the miRNA molecule can silence a native mRNA in the target cell. In some aspects, the polynucleotide is a small interfering RNA (siRNA) or a short hairpin RNA (shRNA) capable of interfering with the expression of an oncogene or other dysregulating polypeptides. In some of these aspects, the siRNA is delivered to the cytoplasm of the target cell, such that the siRNA molecule can silence a native mRNA in the target cell. In some aspects, the polynucleotide is an antisense oligonucleotide (e.g., antisense RNA) that is complementary to an mRNA. In some aspects, the polynucleotide is a long non-coding RNA (lncRNA) capable of regulating gene expression and modulating diseases. In some aspects, the polynucleotide is a DNA that can be transcribed into an RNA. In some of these aspects, the transcribed RNA can be translated into a desired polypeptide.

As used herein, the term “residual nucleic acid molecule” or “residual nucleic acid molecules” refers to contaminating nucleic acids and/or polynucleotides present in a sample comprising an EV, e.g., an exosome. During the preparation of EVs undesired nucleic acid molecules can be present in solution with the EVs. In some aspects, the residual nucleic acid molecules comprise antisense oligomers that failed to associate with the EVs. In some aspects, the residual nucleic acid molecules comprise DNA and/or RNA released by cells during the EV manufacturing process.

As used herein, the term “nanovesicle” refers to an extracellular vesicle with a diameter between 20-250 nm (e.g., between 30-150 nm) and is generated from a cell (e.g., producer cell) by direct or indirect manipulation such that the nanovesicle would not be produced by the cell without the manipulation. Appropriate manipulations of the cell to produce the nanovesicles include but are not limited to serial extrusion, treatment with alkaline solutions, sonication, or combinations thereof. In some aspects, production of nanovesicles can result in the destruction of the producer cell. In some aspects, population of nanovesicles described herein are substantially free of vesicles that are derived from cells by way of direct budding from the plasma membrane or fusion of the late endosome with the plasma membrane. Nanovesicles, once derived from a producer cell, can be isolated from the producer cell based on its size, density, biochemical parameters, or a combination thereof. EVs can be derived from a living or dead organism, explanted tissues or organs, prokaryotic or eukaryotic cells, and/or cultured cells.

The term “microvesicle” or “microparticle,” as used herein, is a type of EV, which is between 50 and 1,000 nanometers (nm) in diameter, and which is found in many types of body fluids as well as the interstitial space between cells. Microvesicles are membrane-bound vesicles containing phospholipids, ranging from 100 nm to 1000 nm shed from almost all cell types. Microvesicles play a role in intercellular communication and can transport mRNA, miRNA, and proteins between cells. They originate directly from the plasma membrane of the cell and reflect the antigenic content of the cells from which they originate. They remove misfolded proteins, cytotoxic agents and metabolic waste from the cell.

The term “microsome,” as used herein, refers to heterogeneous vesicle-like artifacts (˜20-200 nm diameter) re-formed from pieces of the endoplasmic reticulum (ER) when eukaryotic cells are broken-up in the laboratory; microsomes are not present in healthy, living cells. Microsomes can be concentrated and separated from other cellular debris by differential centrifugation. Unbroken cells, nuclei, and mitochondria sediment out at 10,000 g, whereas soluble enzymes and fragmented ER, which contains cytochrome P450 (CYP), remain in solution (g is the Earth's gravitational acceleration). Microsomes have a reddish-brown color, due to the presence of the heme.

As used herein, the terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of desired EVs, that have undergone one or more processes of purification, e.g., a selection or an enrichment of the desired EV preparation. In some aspects, isolating or purifying as used herein is the process of removing, partially removing (e.g., a fraction) the EVs from a sample containing producer cells. In some aspects, an isolated EV composition has no detectable undesired activity or, alternatively, the level or amount of the undesired activity is at or below an acceptable level or amount. In other aspects, an isolated EV composition has an amount and/or concentration of desired EVs at or above an acceptable amount and/or concentration. In other aspects, the isolated EV composition is enriched as compared to the starting material (e.g., producer cell preparations) from which the composition is obtained. This enrichment can be by about 10%, about 20%, about 30%, about 40%, about 50%, about 60%, about 70%, about 80%, about 90%, about 95%, about 96%, about 97%, about 98%, about 99%, about 99.9%, about 99.99%, about 99.999%, about 99.9999%, or greater than about 99.9999% compared to the starting material. In some aspects, isolated EV preparations according to the present disclosure are substantially free of residual contaminating products, including residual biologic products. In some aspects, the isolated EV preparations according to the present disclosure are 100% free, about 99% free, about 98% free, about 97% free, about 96% free, about 95% free, about 94% free, about 93% free, about 92% free, about 91% free, or about 90% free of any contaminating biological matter. Residual contaminating products can include abiotic materials (including chemicals) or unwanted nucleic acids, proteins, lipids, or metabolites. Substantially free of residual biological products can also mean that the EV composition contains no detectable producer cells and that only EVs are detectable.

The term “excipient” refers to an inert substance added to assist in the purification of the EVs. Excipients can modulate the structure of the EV, modulate the adsorption rate of the EVs or the impurities, alter the polarity of the solution being purified, and perform other functions to provide an increase in the purity of the EVs.

As used herein, the term “substantially free” means that a purified composition comprising EVs comprise less than about 10% (m/v) of macromolecules by mass/volume percentage concentration. Some fractions may contain less than about 0.001%, less than about 0.01%, less than about 0.05%, less than about 0.1%, less than about 0.2%, less than about 0.3%, less than about 0.4%, less than about 0.5%, less than about 0.6%, less than about 0.7%, less than about 0.8%, less than about 0.9%, less than about 1%, less than about 2%, less than about 3%, less than about 4%, less than about 5%, less than about 6%, less than about 7%, less than about 8%, less than about 9%, or less than about 10% (m/v) of macromolecules.

As used herein, the term “macromolecule” means a molecule containing a very large number of atoms, such as nucleic acids, proteins, lipids, carbohydrates, metabolites, and/or a combination thereof. In some aspects, “macromolecules” are part of impurities that can be removed during purification as described herein.

The term “nucleic acid molecule” refers to any nucleotide or nucleoside or any polymer or analog thereof, including but not limited to deoxyribonucleic acid (DNA) molecules, ribonucleic acid (RNA) molecules, peptide nucleic acid molecules, locked nucleic acid (LNA) molecules, morpholino nucleic acid molecules, glycol nucleic acid molecules, threose nucleic acid molecules, and any polymers, analogs, or combinations thereof. The term “polynucleotide,” as used herein, refers to a nucleic acid molecule comprising at least two individual nucleotide units.

The term “nuclease” as used herein refers to a protein, e.g., an enzyme that is capable of catalyzing the cleavage of a nucleic acid molecule. In some aspects, the nuclease is an “endonuclease,” which refers to a nuclease that catalyzes cleavage of a nucleic acid molecule between two adjacent nucleotides, wherein at neither of the adjacent nucleotides are at the terminus of the nucleic acid molecule, e.g. an endonuclease catalyzes cleavage between the 5′ and 3′ end of a nucleic acid molecule. Conversely, in some aspects, the nuclease comprises an “exonuclease,” which catalyzes the cleavage of a nucleic acid molecule by removing one or more nucleotides at one or both ends of the nucleic acid molecule, e.g., by removing the 5′ or 3′ nucleotide from the nucleic acid molecule. In some aspects, a nucleic acid molecule is said to be “degraded” following cleavage by a nuclease. Any nuclease known in the art can be used in the methods disclosed herein. In certain aspects, the nuclease is selected from a salt active nuclease (SAN), a benzonase, a denarase, a kryptonase, and any combination thereof. In some aspects, more than one nuclease is applied to the chromatography resins disclosed herein. When more than one nuclease is used, each nuclease can applied to the chromatography resins together, e.g., in a single wash buffer, or each nuclease can be applied to the chromatography resin sequentially.

The terms “anion” and “cation” refer to negatively and positively charged ions, respectively. A “divalent” cation refers to a cation with a valence of 2+. Examples of divalent cations include, but are not limited to, Ca²⁺, Mg²⁺, CO₂⁺, Ni²⁺, Zn²⁺, Ba²⁺, Sr2+, Al²⁺, Ag²⁺, Cu²⁺, and Mn²⁺. A “monovalent cation refers to a cation with a valence of 1+. Examples of monovalent cations include, but are not limited to, Li⁺, K⁺, Na⁺, NH4+, Cu⁺. Examples of anions include, but are not limited to, SCN, Cl⁻, SO₄⁻, and PO₄. In some aspects, the anion and/or the cation (e.g., monovalent cation or divalent cation) can be present in a salt, e.g., a mixture of at least one anion and at least one cation of complementary valences. Any salt comprising an anion or a cation disclosed herein can be used in the methods disclosed herein, e.g., in a nuclease wash buffer disclosed herein. In some aspects, the salt is selected from MgCl₂, Mg(SCN)₂, Mg(SO₄)₂, Mg(PO₄)₂, and any combination thereof.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. In some aspects of the present disclosure, the biologically active molecule attached to the EV is a polypeptide, e.g., an antibody or an antigen binding portion thereof, a fusion protein, a cytokine, or an enzyme.

The term “polypeptide”, as used herein, refers to proteins and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multi-chain polypeptides. Most commonly, disulfide linkages are found in multi-chain polypeptides. The term polypeptide can also apply to amino acid polymers in which one or more amino acid residues are an artificial chemical analog of a corresponding naturally occurring amino acid. In some aspects, a “peptide” can be less than or equal to 50 amino acids long, e.g., about 5, 10, 15, 20, 25, 30, 35, 40, 45, or 50 amino acids long.

A “recombinant” polypeptide or protein refers to a polypeptide or protein produced via recombinant DNA technology. Recombinantly produced polypeptides and proteins expressed in engineered host cells are considered isolated for the purpose of the disclosure, as are native or recombinant polypeptides, which have been separated, fractionated, or partially or substantially purified by any suitable technique. The polypeptides disclosed herein can be recombinantly produced using methods known in the art. Alternatively, the proteins and peptides disclosed herein can be chemically synthesized. In some aspects of the present disclosure, the Scaffold X and/or Scaffold Y proteins present in EVs are recombinantly produced by overexpressing the scaffold proteins in the producer cells, so that levels of scaffold proteins in the resulting EVs are significantly increased with respect to the levels of scaffold proteins present in EVs of producer cells not overexpressing such scaffold proteins.

As used herein, the term “scaffold moiety” refers to a molecule, e.g., a protein such as Scaffold X or Scaffold Y, that can be used to anchor a molecule, e.g., a biologically active molecule, to the EV either on the luminal surface or on the exterior surface of the EV. In certain aspects, a scaffold moiety comprises a synthetic molecule. In some aspects, a scaffold moiety comprises a non-polypeptide moiety. In other aspects, a scaffold moiety comprises, e.g., a lipid, carbohydrate, protein, or combination thereof (e.g., a glycoprotein or a proteolipid) that naturally exists in the EV. In some aspects, a scaffold moiety comprises a lipid, carbohydrate, or protein that does not naturally exist in the EV. In some aspects, a scaffold moiety comprises a lipid or carbohydrate, which naturally exists in the EV but has been enriched in the EV with respect to basal/native/wild type levels. In some aspects, a scaffold moiety comprises a protein which naturally exists in the EV but has been engineered to be enriched in the EV, e.g., by recombinant overexpression in the producer cell, with respect to basal/native/wild type levels. In certain aspects, a scaffold moiety is Scaffold X. In some aspects, a scaffold moiety is Scaffold Y. In further aspects, a scaffold moiety comprises both Scaffold X and Scaffold Y.

As used herein, the term “Scaffold X” or “PrX” refers to EV proteins that have been identified on the surface of EVs. See, e.g., U.S. Pat. No. 10,195,290, which is incorporated herein by reference in its entirety. Non-limiting examples of Scaffold X proteins include: prostaglandin F2 receptor negative regulator (“PTGFRN”); basigin (“BSG”); immunoglobulin superfamily member 2 (“IGSF2”); immunoglobulin superfamily member 3 (“IGSF3”); immunoglobulin superfamily member 8 (“IGSF8”); integrin beta-1 (“ITGB1”); integrin alpha-4 (“ITGA4”); 4F2 cell-surface antigen heavy chain (“SLC3A2”); and a class of ATP transporter proteins (“ATP1A1,” “ATP1A2,” “ATP1A3,” “ATP1A4,” “ATP1B3,” “ATP2B1,” “ATP2B2,” “ATP2B3,” “ATP2B”). In some aspects, a Scaffold X protein can be a whole protein or a fragment thereof (e.g., functional fragment, e.g., the smallest fragment that is capable of anchoring another moiety on the exterior surface or on the luminal surface of the EV). In some aspects, a Scaffold X can anchor a moiety, e.g., a biologically active molecule to the external surface or the luminal surface of the EV. Non-limiting examples of other Scaffold X proteins include e.g., CD13 (aminopeptidase N), MME (membrane metalloendopeptidase), ENPP1 (ectonucleotide pyrophosphatase/phosphodiesterase family member 1), NRP1 (neuropilin-1), CD9, CD63, CD81, PDGFR, GPI anchor proteins, lactadherin, LAMP2, and LAMP2B.

As used herein, the term “Scaffold Y” refers to EV proteins that have been identified within the lumen of EVs. See, e.g., International Publ. Nos. WO/2019/099942 and WO 2020/101740, each of which is incorporated herein by reference in its entirety. Non-limiting examples of Scaffold Y proteins include: myristoylated alanine rich Protein Kinase C substrate (“MARCKS”); myristoylated alanine rich Protein Kinase C substrate like 1 (“MARCKSL1”); and brain acid soluble protein 1 (“BASP1”). In some aspects, a Scaffold Y protein can be a whole protein or a fragment thereof (e.g., functional fragment, e.g., the smallest fragment that is capable of anchoring a moiety on the luminal surface of the EV). In some aspects, a Scaffold Y can anchor a moiety on the luminal surface of the EV. In some aspects of the present disclosure, a moiety can be covalently attached to a Scaffold Y. In some aspects, the moiety can be attached to Scaffold Y on the luminal surface of the EV.

As used herein the term “surface-engineered EV” (e.g., Scaffold X-engineered EV) refers to an EV with the membrane or the surface of the EV modified in its composition so that the surface of the engineered EV is different from that of the EV prior to the modification or of the naturally occurring EV. The engineering can be on the surface of the EV or in the membrane of the EV so that the exterior surface of the EV is changed. For example, the membrane can be modified in its composition of, e.g., a protein, a lipid, a small molecule, a carbohydrate, or a combination thereof. The composition can be changed by a chemical, a physical, or a biological method or by being produced from a cell previously or concurrently modified by a chemical, a physical, or a biological method. Specifically, the composition can be changed by a genetic engineering or by being produced from a cell previously modified by genetic engineering. In some aspects, a surface-engineered EV comprises an exogenous protein (i.e., a protein that the EV does not naturally express) or a fragment or variant thereof that can be exposed to the surface of the EV or can be an anchoring point (attachment) for a moiety exposed on the exterior surface of the EV. In other aspects, a surface-engineered EV comprises a higher expression (e.g., higher number) of a natural EV protein (e.g., Scaffold X) or a fragment or variant thereof that can be exposed to the surface of the EV or is capable of being an anchoring point (attachment) for a moiety exposed on the surface of the EV.

As used herein the term “lumen-engineered exosome” (e.g., Scaffold Y-engineered exosome) refers to an exosome with the membrane or the lumen of the exosome modified in its composition so that the lumen of the engineered exosome is different from that of the exosome prior to the modification or of the naturally occurring exosome. The engineering can be directly on the luminal surface or in the membrane of the exosome so that the lumen of the exosome is changed. For example, the membrane is modified in its composition of a protein, a lipid, a small molecule, a carbohydrate, etc. so that the lumen of the exosome is modified. The composition can be changed by a chemical, a physical, or a biological method or by being produced from a cell previously modified by a chemical, a physical, or a biological method. Specifically, the composition can be changed by a genetic engineering or by being produced from a cell previously modified by genetic engineering. In some aspects, a lumen-engineered exosome comprises an exogenous protein (i.e., a protein that the exosome does not naturally express) or a fragment or variant thereof that can be exposed on the luminal surface of the exosome or can be an anchoring point (attachment) for a moiety exposed on the inner layer of the exosome. In other aspects, a lumen-engineered exosome comprises a higher expression of a natural exosome protein (e.g., Scaffold X or Scaffold Y) or a fragment or variant thereof that can be exposed to the lumen of the exosome or can be an anchoring point (attachment) for a moiety exposed on the luminal surface of the exosome.

As used herein the term “linked to,” “fused,” or “conjugated to” are used interchangeably and refer to a covalent or non-covalent bond formed between a first moiety and a second moiety, e.g., Scaffold X and an antigen, e.g., a scaffold moiety expressed in or on the extracellular vesicle and an antigen, e.g., Scaffold X (e.g., a PTGFRN protein), respectively, in the luminal surface of or on the external surface of the extracellular vesicle. In some aspects, a payload disclosed herein can be directly linked to the exterior surface and/or the luminal surface of an EV (e.g., exosome). As used herein, the term “directly linked,” “directly fused,” or “directly conjugated to” refer to the process of linking (fusing or conjugating) a moiety (e.g., a payload and/or targeting moiety) to the surface of an EV (e.g., exosome) without the use of a scaffold moiety disclosed herein.

As used herein, the term “fusion protein” refers to two or more proteins that are linked or conjugated to each other. For instance, in some aspects, a fusion protein that can be expressed in an EV (e.g., exosome) disclosed herein comprises (i) a payload (e.g., antigen, adjuvant, and/or immune modulator) and (ii) a scaffold moiety (e.g., Scaffold X and/or Scaffold Y). In some aspects, a fusion protein that can be expressed in an EV (e.g., exosome) useful for the present disclosure comprises (i) a targeting moiety and (ii) a scaffold moiety (e.g., Scaffold X and/or Scaffold Y). As described herein, in some aspects, EVs (e.g., exosomes) of the present disclosure can express multiple fusion proteins, wherein a first fusion protein comprises (i) a payload (e.g., antigen, adjuvant, and/or immune modulator) and (ii) a scaffold moiety (e.g., Scaffold X and/or Scaffold Y), and wherein a second fusion protein comprises (i) a targeting moiety and (ii) a scaffold moiety (e.g., Scaffold X and/or Scaffold Y).

### II. Methods of the Present Disclosure

Certain aspects of the present disclosure relate to isolation, purification and/or sub-fractionation of EVs by chromatographic purification methods. Certain aspects of the present disclosure are directed to methods of preparing purified EVs, e.g., exosomes, from a sample comprising EVs, e.g., exosomes, and one or more nucleic acid molecules, comprising: (i) contacting the sample with a chromatography resin and (ii) contacting the chromatography resin with a nuclease wash buffer, wherein the (ii) washing follows the (i) contacting. Some aspects of the present disclosure are directed to methods of reducing the concentration of residual nucleic acid molecule in a sample comprising EVs, e.g., exosomes, comprising (i) contacting the sample with a chromatography resin and (ii) contacting the chromatography resin with a nuclease wash buffer, wherein the (ii) washing follows the (i) contacting. In some aspects, the nuclease wash buffer comprises a nuclease and a cation.

Some aspects of the present disclosure are directed to a method of preparing purified extracellular vesicles (EVs) from a sample comprising EVs and one or more nucleic acid molecules, comprising: (i) contacting the sample with a chromatography resin and (ii) contacting the chromatography resin with a wash buffer comprising a nuclease, a cation, or both a nuclease and a cation; and wherein the (ii) washing follows the (i) contacting.

Some aspects of the present disclosure are directed to a method of reducing the concentration of residual nucleic acid molecule in a sample comprising extracellular vesicles (EVs), comprising (i) contacting the sample with a chromatography resin and (ii) contacting the chromatography resin with a wash buffer comprising a nuclease, a cation, or both a nuclease and a cation; and wherein the (ii) washing follows the (i) contacting.

Some aspects of the present disclosure are directed to a method of preparing purified extracellular vesicles (EVs) from a sample comprising EVs and one or more nucleic acid molecules, comprising: (i) contacting the sample with a chromatography resin; (ii) contacting the chromatography resin with a wash buffer comprising a nuclease, a cation, or both a nuclease and a cation; and (iii) eluting an eluent from the chromatography resin, wherein the eluent comprises the EVs, e.g., exosomes.

Some aspects of the present disclosure are directed to a method of preparing purified extracellular vesicles (EVs) from a sample comprising EVs and one or more nucleic acid molecules, comprising: (i) contacting the sample with a chromatography resin, (ii) eluting an eluent form the chromatography resin, wherein the eluent comprises the EVs, e.g., exosomes, and (iii) contacting the eluent with a nuclease wash buffer. Some aspects of the present disclosure are directed to a method of reducing the concentration of residual nucleic acid molecule in a sample comprising extracellular vesicles (EVs), comprising (i) contacting the sample with a chromatography resin, (ii) eluting an eluent form the chromatography resin, wherein the eluent comprises the EVs, e.g., exosomes, and (iii) contacting the eluent with a nuclease wash buffer. In some aspects, nuclease wash buffer comprises a nuclease and a cation. In some aspects, the eluent is contacted with the nuclease wash buffer by incubation for at least about 1 minute, at least about 2 minutes, at least about 3 minutes, at least about 4 minutes, at least about 5 minutes, at least about 10 minutes, at least about 15 minutes, at least about 20 minutes, at least about 25 minutes, at least about 30 minutes, at least about 60 minutes, at least about 90 minutes, at least about 120 minutes, at least about 180 minutes, at least about 3 hours, at least about 4 hours, at least about 5 hours, at least about 6 hours, at least about 8 hours, at least about 9 hours, at least about 10 hours, at least about 11 hours, at least about 12 hours, at least about 15 hours, at least about 18 hours, at least about 21 hours, or at least about 24 hours. In some aspects, the eluent is contacted with the nuclease wash buffer by incubation for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days. In some aspects, the eluent is contacted with the nuclease wash buffer by incubation at about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., or about 37° C. In some aspects, the eluent is contacted with the nuclease wash buffer by incubation at about 4° C. In some aspects, the eluent is contacted with the nuclease wash buffer by incubation at about 37° C.

In some aspects, the sample comprising EVs, e.g., exosomes, and one or more nucleic acid molecules is contacting with the chromatography resin in a loading buffer. In some aspects, the sample comprises the EVs, e.g., exosomes, and the one or more nucleic acid molecules in a loading buffer.

In some aspects, the flow through following the first wash buffer comprises unbound elements of the loaded sample. In some aspects, the flow through following the nuclease wash buffer comprises fragments of the one or more nucleic acid molecules. In some aspects, the flow through following the second wash buffer comprises fragments of the one or more nucleic acid molecules and nuclease.

In some aspects, one or more of the sample, the first wash buffer, the nuclease wash buffer, the second wash buffer, and the elution buffer is allowed to flow through the chromatography resin by unassisted gravitational flow. In some aspects, one or more of the sample, the first wash buffer, the nuclease wash buffer, the second wash buffer, and the elution buffer is pumped, i.e., by positive pressure applied to a loading site of the chromatography resin or by negative pressure applied at a flow-through site of the chromatography resin, across the chromatography resin at an optimal flow rate. In some aspects, the flow rate is from at least about 0.01 membrane volumes per minute (MV/min) to at least about 5 MV/min, at least about 0.1 MV/min to at least about 5 MV/min, at least about 0.5 MV/min to at least about 5 MV/min, at least about 1 MV/min to at least about 5 MV/min, at least about 0.01 MV/min to at least about 4 MV/min, at least about 0.01 MV/min to at least about 3 MV/min, at least about 0.01 MV/min to at least about 2 MV/min, at least about 0.01 MV/min to at least about 1 MV/min, or at least about 0.01 MV/min to at least about 0.1 MV/min. In some aspects, the flow rate is at least about 0.01 MV/min, at least about 0.05 MV/min, at least about 0.1 MV/min, at least about 0.5 MV/min, at least about 1 MV/min, at least about 2 MV/min, at least about 3 MV/min, at least about 4 MV/min, or at least about 5 MV/min. In certain aspects, the follow rate is at least about 0.1 MV/min. In some aspects, the flow rate during the nuclease wash step is from at least about 0.01 MV/min to at least about 1 MV/min. In some aspects, the flow rate during the nuclease wash step is at least about 0.1 MV/min.

The methods disclosed herein reduce the level, e.g., concentration, of one or more nucleic acid molecules in a sample comprising EVs, e.g., exosomes, and one or more nucleic acid molecules. In some aspects, the sample contacted with the chromatography resin comprises a starting concentration of the one or more nucleic acid molecules, and the eluent comprises an eluted concentration of the one or more nucleic acid molecules, wherein the eluted concentration of the one or more nucleic acid molecules is less than about 10%, less than about 5%, less than about 1%, less than about 0.5%, less than about 0.1%, less than about 0.05%, less than about 0.01%, or less than about 0.001% that of the starting concentration of the one or more nucleic acid molecules.

In some aspects, the reduction in residual nucleic acid molecules is at least about 1000-fold to at least about 100,000-fold. In some aspects, the reduction in residual nucleic acid molecules is at least about 1000-fold. In some aspects, the reduction in residual nucleic acid molecules is at least about 5000-fold. In some aspects, the reduction in residual nucleic acid molecules is at least about 10,000-fold. In some aspects, the reduction in residual nucleic acid molecules is at least about 50,000-fold. In some aspects, the reduction in residual nucleic acid molecules is at least about 100,000-fold.

In some aspects, the eluted concentration of the one or more nucleic acid molecules is less than about 10% that of the starting concentration of the one or more nucleic acid molecules. In some aspects, the eluted concentration of the one or more nucleic acid molecules is less than about 5% that of the starting concentration of the one or more nucleic acid molecules. In some aspects, the eluted concentration of the one or more nucleic acid molecules is less than about 4% that of the starting concentration of the one or more nucleic acid molecules. In some aspects, the eluted concentration of the one or more nucleic acid molecules is less than about 3% that of the starting concentration of the one or more nucleic acid molecules. In some aspects, the eluted concentration of the one or more nucleic acid molecules is less than about 2% that of the starting concentration of the one or more nucleic acid molecules. In some aspects, the eluted concentration of the one or more nucleic acid molecules is less than about 1% that of the starting concentration of the one or more nucleic acid molecules. In some aspects, the eluted concentration of the one or more nucleic acid molecules is less than about 0.5% that of the starting concentration of the one or more nucleic acid molecules. In some aspects, the eluted concentration of the one or more nucleic acid molecules is less than about 0.1% that of the starting concentration of the one or more nucleic acid molecules. In some aspects, the eluted concentration of the one or more nucleic acid molecules is less than about 0.05% that of the starting concentration of the one or more nucleic acid molecules. In certain aspects, the eluted sample comprises no detectable nucleic acid molecules.

II.A. Nuclease Wash Buffer

Certain aspects of the present disclosure are directed to a method comprising contacting a chromatography resin with a nuclease wash buffer. Some aspects of the present disclosure are directed to contacting a solution comprising one or more EVs, e.g., an eluent disclosed herein, with a wash buffer comprising a nuclease, e.g., a nuclease wash buffer. The nuclease wash buffers used herein comprises (i) a nuclease. In some aspects, the nuclease wash buffer further comprises (ii) a cation.

In some aspects, the chromatography resin is contacted with the wash buffer, e.g., the nuclease wash buffer, one time. In some aspects, the chromatography resin is contacted with the wash buffer, e.g., the nuclease wash buffer, at least two times, e.g., the method comprises contacting the chromatography resin with a wash buffer, e.g., a nuclease wash buffer, allowing the nuclease wash buffer to pass through the chromatography resins, and then contacting the chromatography resin with a wash buffer, e.g., a nuclease wash buffer, a second time. In some aspects, the chromatography resin is contacted with the wash buffer, e.g., the nuclease wash buffer, at least three times. In some aspects, the chromatography resin is contacted with the wash buffer, e.g., the nuclease wash buffer, at least four times. In some aspects, the chromatography resin is contacted with the wash buffer, e.g., the nuclease wash buffer, at least five times. In some aspects, the wash buffer, e.g., the nuclease wash buffer, of each contacting is the same. In some aspects, the wash buffer, e.g., the nuclease wash buffer, of each contacting is different.

In some aspects, the wash buffer, e.g., the nuclease wash buffer, is contacted with the chromatography resin, and the flow through is blocked, wherein the wash buffer, e.g., the nuclease wash buffer, remains in contact with the chromatography resin for a period of time. In some aspects, the wash buffer, e.g., the nuclease wash buffer, remains in contact with the chromatography resin for at least about 5 minutes, at least about 10 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, at least about 60 minutes, at least about 75 minutes, at least about 90 minutes, at least about 105 minutes, at least about 120 minutes, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 15 hours, at least about 18 hours, at least about 21 hours, or at least about 24 hours.

In some aspects, a higher concentration of substrate, e.g., residual DNA, increases the rate of enzymatic degradation. In such cases, a lower amount of enzyme can be used, and/or shorter incubation time can be applied. In some aspects, a higher concentration of substrate, e.g., residual DNA, increases the rate of enzymatic degradation, allowing for a shorter incubation time. In some aspects, a shorter incubation time results in decreased degradation of the eluted EVs, e.g., exosomes.

In some aspects, the wash buffer, e.g., the nuclease wash buffer, is contacted with the chromatography resin, and the flow through is collected and contacted again with the chromatography resin. In some aspects, the flow through is circulated back to contact the chromatography resin a second time.

IIA.1. Nucleases

Any nuclease known in the art capable of cleaving and/or degrading nucleic acid molecules can be used in the methods disclosed herein. In some aspects, the nuclease comprises an endonuclease. In some aspects, the nuclease comprises an exonuclease. In some aspects, the nuclease has both endonuclease and exonuclease capabilities. In some aspects, the nuclease is capable of catalyzing the cleavage of RNA, DNA, or both RNA and DNA. In some aspects, the nuclease is capable of catalyzing the cleavage of single stranded or double stranded nucleic acid molecules, e.g., double stranded DNA (dsDNA) or single stranded (ssDNA). In some aspects, the nuclease is capable of catalyzing the cleavage of single stranded and double stranded nucleic acid molecules, e.g., dsDNA or ssDNA.

In some aspects, the nuclease comprises a genetically engineered nuclease. In some aspects, the nuclease comprises a recombinant nuclease. In some aspects, the nuclease comprises a salt-tolerant nuclease (e.g., a salt active nuclease or a KRYPTONASE™). In certain aspects, the nuclease comprises a KRYPTONASE™.

In certain aspects, the nuclease comprises a salt active nuclease (SAN). SAN is a general, unspecific endonuclease that cleaves dsDNA, ssDNA, and RNA. SAN is active at above neutral pH, with an optimal range between pH 8.5-9.5; and it has optimum activity at high concentrations of salt at high pH. The end-products upon complete degradation of, e.g., DNA comprise of a majority of oligo nucleotides of about 5 nucleotides in length. SAN is known to be highly active over the temperature range 10-40° C., with an optimal NaCl-concentration for activity of about 0.5 M. However, SAN remains active in low salt buffers, as well. Mg2+ (>1 mM) is required for activity.

In some aspects, the nuclease comprises a Serratia marcescens-derived nuclease (e.g., BENSONASE® or DENARASE®). In some aspects, the nuclease comprises BENSONASE®. BENSONASE® is a genetically engineered endonuclease from Serratia marcescens. The protein is a dimer of 30 kDa subunits with two essential disulfide bonds. This endonuclease attacks and degrades all forms of DNA and RNA (single stranded, double stranded, linear, and circular) and is effective over a wide range of operating conditions. The optimum pH for enzyme activity is found to be about 8.0 to about 9.2. BENSONASE® digests nucleic acids to 5′-monophosphate terminated oligonucleotides, about 3 to about 5 bases in length.

In some aspects, the nuclease comprises DENARASE®. DENARASE® is a genetically engineered endonuclease from Serratia marcescens, which specifically hydrolyzes the phosphodiester bonds between nucleotides leaving smaller fragments of about 5 to about 8 nucleotides in length. The enzyme is active on all forms of nucleic acids including single-stranded, double-stranded, linear, circular or supercoiled.

In some aspects, the nuclease wash buffer comprises at least two, at least three, at least four, or at least five nucleases. In certain aspects, the wash buffer comprises two nucleases. In some aspects, the nuclease wash buffer comprises an endonuclease and an exonuclease. In some aspects, the nuclease wash buffer comprises at least two endonucleases. In some aspects, the nuclease wash buffer comprises at least two exonucleases. In some aspects, the nuclease wash buffer comprises a first salt-tolerant nuclease and a second salt-tolerant nuclease. In some aspects, the nuclease wash buffer comprises a SAN and a KRYPTONASE™. In some aspects, the nuclease wash buffer comprises a salt-tolerant nuclease and a genetically modified nuclease. In some aspects, the nuclease wash buffer comprises a salt-tolerant nuclease and a Serratia marcescens-derived nuclease. In some aspects, the nuclease wash buffer comprises a salt-tolerant nuclease and a BENSONASE®. In some aspects, the nuclease wash buffer comprises a salt-tolerant nuclease and a DENARASE®. In some aspects, the nuclease wash buffer comprises a SAN and a BENSONASE®. In some aspects, the nuclease wash buffer comprises a SAN and a DENARASE®. In some aspects, the nuclease wash buffer comprises a KRYPTONASE™ and a BENSONASE®. In some aspects, the nuclease wash buffer comprises a KRYPTONASE™ and a DENARASE®. In some aspects, the nuclease wash buffer comprises at least two Serratia marcescens-derived nucleases. In some aspects, the nuclease wash buffer comprises a BENSONASE® and a DENARASE®.

In some aspects, the nuclease wash buffer comprises at least three nucleases. In some aspects, the nuclease wash buffer comprises a SAN, a BENSONASE®, and at least one additional nuclease. In some aspects, the nuclease wash buffer comprises a SAN, a DENARASE® and at least one additional nuclease. In some aspects, the nuclease wash buffer comprises a SAN, a BENSONASE®, and a DENARASE®. In some aspects, the nuclease wash buffer comprises a KRYPTONASE™, a BENSONASE®, and a DENARASE®.

In some aspects, the nuclease wash buffer comprises at least four nucleases. In some aspects, the nuclease wash buffer comprises a SAN, a BENSONASE®, a DENARASE®, and at least one additional nuclease. In some aspects, the nuclease wash buffer comprises a KRYPTONASE™, a BENSONASE®, a DENARASE®, and at least one additional nuclease. In some aspects, the nuclease wash buffer comprises a SAN, a KRYPTONASE™, a BENSONASE®, and a DENARASE®.

In some aspects, the nuclease wash buffer comprises at least five nucleases. In some aspects, the nuclease wash buffer comprises a SAN, a KRYPTONASE™, a BENSONASE®, a DENARASE®, and at least one additional nuclease.

In some aspects, the nuclease wash buffer comprises at least about 1 unit/mL to at least about 500 units/mL of the nuclease, e.g., the SAN, KRYPTONASE™, BENSONASE®, DENARASE®, or any combination thereof. In some aspects, the nuclease wash buffer comprises at least about 1 unit/mL to at least about 100 units/mL, at least about 1 unit/mL to at least about 75 units/mL, at least about 1 unit/mL to at least about 50 units/mL, at least about 10 units/mL to at least about 100 units/mL, at least about 10 units/mL to at least about 75 units/mL, at least about 10 units/mL to at least about 50 units/mL, at least about 20 units/mL to at least about 100 units/mL, at least about 20 units/mL to at least about 75 units/mL, at least about 20 units/mL to at least about 50 units/mL, at least about 30 units/mL to at least about 100 units/mL, at least about 30 units/mL to at least about 75 units/mL, at least about 30 units/mL to at least about 50 units/mL, at least about 40 units/mL to at least about 100 units/mL, at least about 40 units/mL to at least about 75 units/mL, at least about 40 units/mL to at least about 50 units/mL, at least about 50 units/mL to at least about 100 units/mL, or at least about 50 units/mL to at least about 75 units/mL of the nuclease, e.g., the SAN, KRYPTONASE™, BENSONASE®, DENARASE®, or any combination thereof. In some aspects, the nuclease wash buffer comprises at least about 1 unit/mL, at least about 5 units/mL, at least about 10 units/mL, at least about 15 units/mL, at least about 20 units/mL, at least about 25 units/mL, at least about 30 units/mL, at least about 35 units/mL, at least about 40 units/mL, at least about 45 units/mL, at least about 50 units/mL, at least about 60 units/mL, at least about 65 units/mL, at least about 70 units/mL, at least about 80 units/mL, at least about 90 units/mL, or at least about 100 unit/mL of the nuclease, e.g., the SAN, KRYPTONASE™, BENSONASE®, DENARASE®, or any combination thereof.

In certain aspects, the nuclease wash buffer comprises at least about 1 unit/mL to at least about 100 units/mL SAN. In certain aspects, the nuclease wash buffer comprises at least about 1 unit/mL, at least about 5 units/mL, at least about 10 units/mL, at least about 15 units/mL, at least about 20 units/mL, at least about 25 units/mL, at least about 30 units/mL, at least about 35 units/mL, at least about 40 units/mL, at least about 45 units/mL, at least about 50 units/mL, at least about 60 units/mL, at least about 65 units/mL, at least about 70 units/mL, at least about 80 units/mL, at least about 90 units/mL, or at least about 100 unit/mL SAN. In particular aspects, the nuclease wash buffer comprises at least about 40 units/mL SAN.

In certain aspects, the nuclease wash buffer comprises at least about 1 unit/mL to at least about 100 units/mL KRYPTONASE™. In certain aspects, the nuclease wash buffer comprises at least about 1 unit/mL, at least about 5 units/mL, at least about 10 units/mL, at least about 15 units/mL, at least about 20 units/mL, at least about 25 units/mL, at least about 30 units/mL, at least about 35 units/mL, at least about 40 units/mL, at least about 45 units/mL, at least about 50 units/mL, at least about 60 units/mL, at least about 65 units/mL, at least about 70 units/mL, at least about 80 units/mL, at least about 90 units/mL, or at least about 100 unit/mL KRYPTONASE™. In particular aspects, the nuclease wash buffer comprises at least about 40 units/mL KRYPTONASE™.

In certain aspects, the nuclease wash buffer comprises at least about 1 unit/mL to at least about 100 units/mL BENSONASE®. In certain aspects, the nuclease wash buffer comprises at least about 1 unit/mL, at least about 5 units/mL, at least about 10 units/mL, at least about 15 units/mL, at least about 20 units/mL, at least about 25 units/mL, at least about 30 units/mL, at least about 35 units/mL, at least about 40 units/mL, at least about 45 units/mL, at least about 50 units/mL, at least about 60 units/mL, at least about 65 units/mL, at least about 70 units/mL, at least about 80 units/mL, at least about 90 units/mL, or at least about 100 unit/mL BENSONASE®. In particular aspects, the nuclease wash buffer comprises at least about 40 units/mL BENSONASE®.

In certain aspects, the nuclease wash buffer comprises at least about 1 unit/mL to at least about 100 units/mL DENARASE®. In certain aspects, the nuclease wash buffer comprises at least about 1 unit/mL, at least about 5 units/mL, at least about 10 units/mL, at least about 15 units/mL, at least about 20 units/mL, at least about 25 units/mL, at least about 30 units/mL, at least about 35 units/mL, at least about 40 units/mL, at least about 45 units/mL, at least about 50 units/mL, at least about 60 units/mL, at least about 65 units/mL, at least about 70 units/mL, at least about 80 units/mL, at least about 90 units/mL, or at least about 100 unit/mL DENARASE®. In particular aspects, the nuclease wash buffer comprises at least about 40 units/mL DENARASE®.

As used herein, “units/mL” or an enzyme, e.g., a nuclease, e.g., SAN, refers to an enzyme unit (per mL), wherein one unit is the amount of an enzyme that catalyzes the conversion of one micromole of substrate per minute under optimal conditions. As such, the exact amount of enzyme (e.g., mol of enzyme) will vary depending on the enzyme. In some aspects, the unit is a standard Kunitz unit. In some aspects, the unit is as-defined by the manufacturer of the enzyme.

In some aspects, the method comprises (i) contacting the sample with a chromatography resin and (ii) contacting the chromatography resin with a nuclease wash buffer; wherein the nuclease wash buffer comprises a nuclease; and wherein the (ii) washing follows the (i) contacting. In some aspects, the sample comprising the EVs, e.g., exosomes, is contacted with a second nuclease treatment at one or more additional stage. In some aspects, the sample comprising the EVs, e.g., exosomes, is contacted with a second nuclease treatment prior to (i) contacting the sample with a chromatography resin. In some aspects, the method comprises (i) contacting the sample with a chromatography resin, (ii) contacting the chromatography resin with a nuclease wash buffer, (iii) eluting an eluent from the chromatography resin, wherein the eluent comprises the EVs, e.g., exosomes, and (iv) contacting the eluent with a second nuclease treatment. In some aspects, the second nuclease treatment and the nuclease wash buffer comprise the same nuclease. In some aspects, the second nuclease treatment and the nuclease wash buffer comprises different nucleases.

II.A.2. Cation

Any cation can be used in the wash buffers, e.g., the nuclease wash buffers, disclosed herein. In some aspects, the cation is a monovalent cation. In some aspects, the monovalent cation is selected from Li⁺, K⁺, Na⁺, NH4′, Cu⁺, and any combination thereof. In some aspects, the nuclease wash buffer comprises Li⁺. In some aspects, the nuclease wash buffer comprises K⁺. In some aspects, the nuclease wash buffer comprises Na⁺. In some aspects, the nuclease wash buffer comprises NH4⁺. In some aspects, the nuclease wash buffer comprises Cu⁺.

In some aspects, the cation is a divalent cation. In some aspects, the divalent cation is selected from Ca²⁺, Mg²⁺, Co²⁺, Ni²⁺, Zn²⁺, Ba²⁺, Sr²⁺, Al²⁺, Ag²⁺, Cu²⁺, Mn²⁺, and any combination thereof. In some aspects, the nuclease wash buffer comprises Ca²⁺. In some aspects, the nuclease wash buffer comprises Mg²⁺. In some aspects, the nuclease wash buffer comprises Co²⁺. In some aspects, the nuclease wash buffer comprises Ni²⁺. In some aspects, the nuclease wash buffer comprises Zn²⁺. In some aspects, the nuclease wash buffer comprises Ba²⁺. In some aspects, the nuclease wash buffer comprises Sr²⁺. In some aspects, the nuclease wash buffer comprises Al²⁺. In some aspects, the nuclease wash buffer comprises Ag²⁺. In some aspects, the nuclease wash buffer comprises Cu²⁺. In some aspects, the nuclease wash buffer comprises Mn²⁺.

In some aspects, the cation is associated with an anion, e.g., in a salt. In some aspects, the nuclease wash buffer comprises a salt, wherein the salt comprises a cation disclosed herein and an anion. In some aspects, the salt comprises a cation disclosed herein and an anion selected from SCN⁻, Cl⁻, SO₄⁻, PO₄⁻, and any combination thereof. In some aspects, the salt comprises a cation disclosed herein and SCN⁻. In some aspects, the salt comprises a cation disclosed herein and Cl⁻. In some aspects, the salt comprises a cation disclosed herein and SO₄. In some aspects, the salt comprises a cation disclosed herein and PO₄.

In some aspects, the wash buffer, e.g., the nuclease wash buffer, comprises MgCl₂, Mg(SCN)₂, Mg(SO₄)₂, Mg(PO₄)₂, or any combination thereof. In some aspects, the wash buffer, e.g., the nuclease wash buffer, comprises MgCl₂. In some aspects, the wash buffer, e.g., the nuclease wash buffer, comprises Mg(SCN)₂. In some aspects, the wash buffer, e.g., the nuclease wash buffer, comprises Mg(SO₄)₂. In some aspects, the wash buffer, e.g., the nuclease wash buffer, comprises Mg(PO₄)₂.

In some aspects, the wash buffer, e.g., the nuclease wash buffer, comprises CaCl₂), Ca(SCN)₂, Ca(SO₄)₂, Ca(PO₄)₂, or any combination thereof. In some aspects, the wash buffer, e.g., the nuclease wash buffer, comprises CaCl₂). In some aspects, the wash buffer, e.g., the nuclease wash buffer, comprises Ca(SCN)₂. In some aspects, the wash buffer, e.g., the nuclease wash buffer, comprises In some aspects, the wash buffer, e.g., the nuclease wash buffer, comprises Ca(PO₄)₂.

In some aspects, the wash buffer, e.g., the nuclease wash buffer, comprises CoCl₂, Co(SCN)₂, Co(SO₄)₂, Co(PO₄)₂, or any combination thereof. In some aspects, the wash buffer, e.g., the nuclease wash buffer, comprises CoCl₂. In some aspects, the wash buffer, e.g., the nuclease wash buffer, comprises Co(SCN)₂. In some aspects, the wash buffer, e.g., the nuclease wash buffer, comprises Co(SO₄)₂. In some aspects, the wash buffer, e.g., the nuclease wash buffer, comprises Co(PO₄)₂.

In some aspects, the wash buffer, e.g., the nuclease wash buffer, comprises NiCl₂, Ni(SCN)₂, Ni(SO₄)₂, Ni(PO₄)₂, or any combination thereof.

In some aspects, the wash buffer, e.g., the nuclease wash buffer, comprises ZnCl₂, Zn(SCN)₂, Zn(SO₄)₂, Zn(PO₄)₂, or any combination thereof. In some aspects, the wash buffer, e.g., the nuclease wash buffer, comprises ZnCl₂. In some aspects, the wash buffer, e.g., the nuclease wash buffer, comprises Zn(SCN)₂. In some aspects, the wash buffer, e.g., the nuclease wash buffer, comprises Zn(SO₄)₂. In some aspects, the wash buffer, e.g., the nuclease wash buffer, comprises Zn(PO₄)₂.

In some aspects, the wash buffer, e.g., the nuclease wash buffer, comprises BaCl₂, Ba(SCN)₂, Ba(SO₄)₂, Ba(PO₄)₂, or any combination thereof. In some aspects, the wash buffer, e.g., the nuclease wash buffer, comprises BaCl₂. In some aspects, the wash buffer, e.g., the nuclease wash buffer, comprises Ba(SCN)₂. In some aspects, the wash buffer, e.g., the nuclease wash buffer, comprises Ba(SO₄)₂. In some aspects, the wash buffer, e.g., the nuclease wash buffer, comprises Ba(PO₄)₂.

In some aspects, the wash buffer, e.g., the nuclease wash buffer, comprises SrCl₂, Sr(SCN)₂, Sr(SO₄)₂, Sr(PO₄)₂, or any combination thereof. In some aspects, the wash buffer, e.g., the nuclease wash buffer, comprises SrCl₂. In some aspects, the wash buffer, e.g., the nuclease wash buffer, comprises Sr(SCN)₂. In some aspects, the wash buffer, e.g., the nuclease wash buffer, comprises Sr(SO₄)₂. In some aspects, the wash buffer, e.g., the nuclease wash buffer, comprises Sr(PO₄)₂.

In some aspects, the wash buffer, e.g., the nuclease wash buffer, comprises AlCl₂, Al(SCN)₂, Al(SO₄)₂, Al(PO₄)₂, or any combination thereof. In some aspects, the wash buffer, e.g., the nuclease wash buffer, comprises AlCl₂. In some aspects, the wash buffer, e.g., the nuclease wash buffer, comprises Al(SCN)₂. In some aspects, the wash buffer, e.g., the nuclease wash buffer, comprises Al(SO₄)₂. In some aspects, the wash buffer, e.g., the nuclease wash buffer, comprises Al(PO₄)₂.

In some aspects, the wash buffer, e.g., the nuclease wash buffer, comprises AgCl₂, Ag(SCN)₂, Ag(SO₄)₂, Ag(PO₄)₂, or any combination thereof. In some aspects, the wash buffer, e.g., the nuclease wash buffer, comprises AgCl₂. In some aspects, the wash buffer, e.g., the nuclease wash buffer, comprises Ag(SCN)₂. In some aspects, the wash buffer, e.g., the nuclease wash buffer, comprises Ag(SO₄)₂. In some aspects, the wash buffer, e.g., the nuclease wash buffer, comprises Ag(PO₄)₂.

In some aspects, the wash buffer, e.g., the nuclease wash buffer, comprises CuCl₂, Cu(SCN)₂, Cu(SO₄)₂, Cu(PO₄)₂, or any combination thereof. In some aspects, the wash buffer, e.g., the nuclease wash buffer, comprises CuCl₂. In some aspects, the wash buffer, e.g., the nuclease wash buffer, comprises Cu(SCN)₂. In some aspects, the wash buffer, e.g., the nuclease wash buffer, comprises Cu(SO₄)₂. In some aspects, the wash buffer, e.g., the nuclease wash buffer, comprises Cu(PO₄)₂.

In some aspects, the wash buffer, e.g., the nuclease wash buffer, comprises MnCl₂, Mn(SCN)₂, Mn(SO₄)₂, Mn(PO₄)₂, or any combination thereof. In some aspects, the wash buffer, e.g., the nuclease wash buffer, comprises MnCl₂. In some aspects, the wash buffer, e.g., the nuclease wash buffer, comprises Mn(SCN)₂. In some aspects, the wash buffer, e.g., the nuclease wash buffer, comprises Mn(SO₄)₂. In some aspects, the wash buffer, e.g., the nuclease wash buffer, comprises Mn(PO₄)₂.

In some aspects, the wash buffer, e.g., the nuclease wash buffer, comprises at least about 0.001 M to at least about 1.0 M of the cation, e.g., Mg²⁺. In some aspects, the nuclease wash buffer comprises at least about 0.001 M, at least about 0.002 M, at least about 0.003 M, at least about 0.004 M, at least about 0.005 M, at least about 0.007 M, at least about 0.008 M, at least about 0.009 M, at least about 0.01 M, at least about 0.02 M, at least about 0.03 M, at least about 0.04 M, at least about 0.05 M, at least about 0.06 M, at least about 0.07 M, at least about 0.08 M, at least about 0.09 M, at least about 0.1 M, at least about 0.15 M, at least about 0.2 M, at least about 0.25 M, at least about 0.3 M, at least about 0.35 M, at least about 0.4 M, at least about 0.45 M, at least about 0.5 M, at least about 0.55 M, at least about 0.6 M, at least about 0.65 M, at least about 0.7 M, at least about 0.75 M, at least about 0.8 M, at least about 0.85 M, at least about 0.9 M, at least about 0.95 M, or at least about 1.0 M of the cation, e.g., Mg²⁺.

In certain aspects, the cation comprises Mg²⁺, and the concentration of the Mg²⁺ in the wash buffer, e.g., the nuclease wash buffer, is at least about 0.001 M, at least about 0.002 M, at least about 0.003 M, at least about 0.004 M, at least about 0.005 M, at least about 0.007 M, at least about 0.008 M, at least about 0.009 M, at least about 0.01 M, at least about 0.02 M, at least about 0.03 M, at least about 0.04 M, at least about 0.05 M, at least about 0.06 M, at least about 0.07 M, at least about 0.08 M, at least about 0.09 M, at least about 0.1 M, at least about 0.15 M, at least about 0.2 M, at least about 0.25 M, at least about 0.3 M, at least about 0.35 M, at least about 0.4 M, at least about 0.45 M, at least about 0.5 M, at least about 0.55 M, at least about 0.6 M, at least about 0.65 M, at least about 0.7 M, at least about 0.75 M, at least about 0.8 M, at least about 0.85 M, at least about 0.9 M, at least about 0.95 M, or at least about 1.0 M Mg²⁺. In some aspects, the concentration of the Mg²⁺ in the wash buffer, e.g., the nuclease wash buffer, is at least about 0.001 M Mg²⁺. In some aspects, the concentration of the Mg²⁺ in the wash buffer, e.g., the nuclease wash buffer, is at least about 0.002 M Mg²⁺. In some aspects, the concentration of the Mg²⁺ in the wash buffer, e.g., the nuclease wash buffer, is at least about 0.003 M Mg²⁺. In some aspects, the concentration of the Mg²⁺ in the wash buffer, e.g., the nuclease wash buffer, is at least about 0.004 M Mg²⁺. In some aspects, the concentration of the Mg²⁺ in the wash buffer, e.g., the nuclease wash buffer, is at least about 0.005 M Mg²⁺. In some aspects, the concentration of the Mg²⁺ in the wash buffer, e.g., the nuclease wash buffer, is at least about 0.20 M Mg²⁺. In some aspects, the concentration of the Mg²⁺ in the wash buffer, e.g., the nuclease wash buffer, is at least about 0.25 M Mg²⁺. In some aspects, the concentration of the Mg²⁺ in the wash buffer, e.g., the nuclease wash buffer, is at least about 0.30 M Mg²⁺. In some aspects, the concentration of the Mg²⁺ in the wash buffer, e.g., the nuclease wash buffer, is at least about 0.35 M Mg²⁺. In some aspects, the concentration of the Mg²⁺ in the wash buffer, e.g., the nuclease wash buffer, is at least about 0.40 M Mg²⁺. In some aspects, the concentration of the Mg²⁺ in the wash buffer, e.g., the nuclease wash buffer, is at least about 0.45 M Mg²⁺. In some aspects, the concentration of the Mg²⁺ in the wash buffer, e.g., the nuclease wash buffer, is at least about 0.50 M Mg²⁺. In some aspects, the wash buffer, e.g., the nuclease wash buffer, comprises at least about 0.35 M MgCl₂.

In certain aspects, the cation comprises Ca²⁺, and the concentration of the Ca²⁺ in the wash buffer, e.g., the nuclease wash buffer, is at least about 0.001 M, at least about 0.002 M, at least about 0.003 M, at least about 0.004 M, at least about 0.005 M, at least about 0.007 M, at least about 0.008 M, at least about 0.009 M, at least about 0.01 M, at least about 0.02 M, at least about 0.03 M, at least about 0.04 M, at least about 0.05 M, at least about 0.06 M, at least about 0.07 M, at least about 0.08 M, at least about 0.09 M, at least about 0.1 M, at least about 0.15 M, at least about 0.2 M, at least about 0.25 M, at least about 0.3 M, at least about 0.35 M, at least about 0.4 M, at least about 0.45 M, at least about 0.5 M, at least about 0.55 M, at least about 0.6 M, at least about 0.65 M, at least about 0.7 M, at least about 0.75 M, at least about 0.8 M, at least about 0.85 M, at least about 0.9 M, at least about 0.95 M, or at least about 1.0 M Ca²⁺. In some aspects, the concentration of the Ca²⁺ in the wash buffer, e.g., the nuclease wash buffer, is at least about 0.001 M Ca²⁺. In some aspects, the concentration of the Ca²⁺ in the wash buffer, e.g., the nuclease wash buffer, is at least about 0.002 M Ca²⁺. In some aspects, the concentration of the Ca²⁺ in the wash buffer, e.g., the nuclease wash buffer, is at least about 0.003 M Ca²⁺. In some aspects, the concentration of the Ca²⁺ in the wash buffer, e.g., the nuclease wash buffer, is at least about 0.004 M Ca²⁺. In some aspects, the concentration of the Ca²⁺ in the wash buffer, e.g., the nuclease wash buffer, is at least about 0.005 M Ca²⁺. In some aspects, the concentration of the Ca²⁺ in the wash buffer, e.g., the nuclease wash buffer, is at least about 0.20 M Ca²⁺. In some aspects, the concentration of the Ca²⁺ in the wash buffer, e.g., the nuclease wash buffer, is at least about 0.25 M Ca²⁺. In some aspects, the concentration of the Ca²⁺ in the wash buffer, e.g., the nuclease wash buffer, is at least about 0.30 M Ca²⁺. In some aspects, the concentration of the Ca²⁺ in the wash buffer, e.g., the nuclease wash buffer, is at least about 0.35 M Ca²⁺. In some aspects, the concentration of the Ca²⁺ in the wash buffer, e.g., the nuclease wash buffer, is at least about 0.40 M Ca²⁺. In some aspects, the concentration of the Ca²⁺ in the wash buffer, e.g., the nuclease wash buffer, is at least about 0.45 M Ca²⁺. In some aspects, the concentration of the Ca²⁺ in the wash buffer, e.g., the nuclease wash buffer, is at least about 0.50 M Ca²⁺. In some aspects, the wash buffer, e.g., the nuclease wash buffer, comprises at least about 0.35 M CaCl₂).

II.B. Chromatography Resins

Certain aspects of the present disclosure are directed to methods of preparing purified EVs, e.g., exosomes, from a sample comprising EVs, e.g., exosomes, and one or more nucleic acid molecules, comprising: (i) contacting the sample with a chromatography resin and (ii) contacting the chromatography resin with a wash buffer, e.g., a nuclease wash buffer, wherein the (ii) washing follows the (i) contacting. Other aspects of the present disclosure are directed to methods of preparing purified EVs from a sample comprising EVs and one or more nucleic acid molecules, comprising: (i) contacting the sample with a chromatography resin, (ii) eluting an eluent form the chromatography resin, wherein the eluent comprises the EVs, e.g., exosomes, and (iii) contacting the eluent with a nuclease wash buffer. These methods reduce the level of contaminant nucleic acid molecules in a sample comprising EVs, e.g., exosomes, and one or more nucleic acid molecules. As such, the methods disclosed herein can be performed using any chromatography resin.

In some aspects, the chromatography resin comprises an ion exchange chromatography resin. In certain aspects, the chromatography resin comprises an anion exchange (AEX) resin. In some aspects, the chromatography resin comprises a cation exchange (CEX) resin. In some aspects, the chromatography resin comprises a hydrophobic interaction resin. In some aspects, the chromatography resin comprises a hydrophobic charge induction chromatography resin. In some aspects, the chromatography resin comprises a mixed mode resin. In some aspects, the chromatography resin comprises an immobilized metal affinity resin. In some aspects, the chromatography resin comprise a ceramic hydroxyapatite resin. In some aspects, the chromatography resin comprise a fluoro hydroxyapatite resin. In some aspects, the chromatography resin comprise a combination of one or more chromatography resin disclosed herein. In some aspects, the chromatography resin comprises a mixed-mode chromatography (MMC) resin.

In certain aspects, the chromatography resin comprises an ion exchange chromatography resin (e.g. an AEX or a CEX chromatography resin), and the nuclease wash buffer comprises at least about 0.001 M to at least about 1.0 M of the cation, e.g., Mg²⁺ or Ca²⁺. In some aspects, the chromatography resin comprises an ion exchange chromatography resin (e.g. an AEX or a CEX chromatography resin), and the nuclease wash buffer comprises at least about 0.1 M of the cation, e.g., Mg²⁺ or Ca²⁺. In some aspects, the chromatography resin comprises an ion exchange chromatography resin (e.g. an AEX or a CEX chromatography resin), and the nuclease wash buffer comprises at least about 0.15 M of the cation, e.g., Mg²⁺ or Ca²⁺. In some aspects, the chromatography resin comprises an ion exchange chromatography resin (e.g. an AEX or a CEX chromatography resin), and the nuclease wash buffer comprises at least about 0.2 M of the cation, e.g., Mg²⁺ or Ca²⁺. In some aspects, the chromatography resin comprises an ion exchange chromatography resin (e.g. an AEX or a CEX chromatography resin), and the nuclease wash buffer comprises at least about 0.25 M of the cation, e.g., Mg²⁺ or Ca²⁺. In some aspects, the chromatography resin comprises an ion exchange chromatography resin (e.g. an AEX or a CEX chromatography resin), and the nuclease wash buffer comprises at least about 0.3 M of the cation, e.g., Mg²⁺ or Ca²⁺. In some aspects, the chromatography resin comprises an ion exchange chromatography resin (e.g. an AEX or a CEX chromatography resin), and the nuclease wash buffer comprises at least about 0.35 M of the cation, e.g., Mg²⁺ or Ca²⁺. In some aspects, the chromatography resin comprises an ion exchange chromatography resin (e.g. an AEX or a CEX chromatography resin), and the nuclease wash buffer comprises at least about 0.4 M of the cation, e.g., Mg²⁺ or Ca²⁺. In some aspects, the chromatography resin comprises an ion exchange chromatography resin (e.g. an AEX or a CEX chromatography resin), and the nuclease wash buffer comprises at least about 0.45 M of the cation, e.g., Mg²⁺ or Ca²⁺. In some aspects, the chromatography resin comprises an ion exchange chromatography resin (e.g. an AEX or a CEX chromatography resin), and the nuclease wash buffer comprises at least about 0.5 M of the cation, e.g., Mg²⁺ or Ca²⁺. In some aspects, the chromatography resin comprises an ion exchange chromatography resin (e.g. an AEX or a CEX chromatography resin), and the nuclease wash buffer comprises at least about 0.6 M of the cation, e.g., Mg²⁺ or Ca²⁺. In some aspects, the chromatography resin comprises an ion exchange chromatography resin (e.g. an AEX or a CEX chromatography resin), and the nuclease wash buffer comprises at least about 0.7 M of the cation, e.g., Mg²⁺ or Ca²⁺. In some aspects, the chromatography resin comprises an ion exchange chromatography resin (e.g. an AEX or a CEX chromatography resin), and the nuclease wash buffer comprises at least about 0.8 M of the cation, e.g., Mg²⁺ or Ca²⁺. In some aspects, the chromatography resin comprises an ion exchange chromatography resin (e.g. an AEX or a CEX chromatography resin), and the nuclease wash buffer comprises at least about 0.9 M of the cation, e.g., Mg²⁺ or Ca²⁺. In some aspects, the chromatography resin comprises an ion exchange chromatography resin (e.g. an AEX or a CEX chromatography resin), and the nuclease wash buffer comprises at least about 1.0 M of the cation, e.g., Mg²⁺ or Ca²⁺.

In certain aspects, the method comprises (i) contacting the sample with an AEX chromatography resin and (ii) contacting the chromatography resin with a nuclease wash buffer. In some aspects, the method further comprises subjecting the sample to one or more additional chromatography resins. In some aspects, the one or more additional chromatography resins comprises an additional AEX resin, a CEX resin, an MMC resin, a hydrophobic charge induction chromatography resin, a hydrophobic interaction chromatography resin, an immobilized metal affinity resin, or any combination thereof. In some aspects, the sample is contacted with a CEX resin after the AEX resin. In some aspects, wherein the sample is contacted with an MMC resin after the CEX resin. In some aspects, the sample is contacted with an MMC resin after the AEX resin. In certain aspects, the sample is contacted with (i) the AEX resin, (ii) a CEX resin, and (iii) an MMC resin, in the sequence (i), (ii), then (iii).

In some aspects, the sample is contacted with (a) an AEX resin, (b) a CEX resin, and (c) an MMC resin; wherein after the sample is contacted with the AEX resin, the AEX resin is contacted with a nuclease wash buffer. In some aspects, the sample is contacted with (a) an AEX resin, (b) a CEX resin, and (c) an MMC resin; wherein after the sample is contacted with the CEX resin, the CEX resin is contacted with a nuclease wash buffer. In some aspects, the sample is contacted with (a) an AEX resin, (b) a CEX resin, and (c) an MMC resin; wherein after the sample is contacted with the MMC resin, the MMC resin is contacted with a nuclease wash buffer.

In some aspects, the sample is contacted with (a) an AEX resin, (b) a CEX resin, and (c) an MMC resin; wherein (i) after the sample is contacted with the AEX resin, the AEX resin is contacted with a nuclease wash buffer; and wherein (ii) after the sample is contacted with the CEX resin, the CEX resin is contacted with a nuclease wash buffer. In some aspects, the sample is contacted with (a) an AEX resin, (b) a CEX resin, and (c) an MMC resin; wherein (i) after the sample is contacted with the AEX resin, the AEX resin is contacted with a nuclease wash buffer; and wherein (ii) after the sample is contacted with the MMC resin, the MMC resin is contacted with a nuclease wash buffer. In some aspects, the sample is contacted with (a) an AEX resin, (b) a CEX resin, and (c) an MMC resin; wherein (i) after the sample is contacted with the CEX resin, the CEX resin is contacted with a nuclease wash buffer; and wherein (ii) after the sample is contacted with the MMC resin, the MMC resin is contacted with a nuclease wash buffer. In some aspects, the sample is contacted with (a) an AEX resin, (b) a CEX resin, and (c) an MMC resin; wherein (i) after the sample is contacted with the AEX resin, the AEX resin is contacted with a nuclease wash buffer; wherein (ii) after the sample is contacted with the CEX resin, the CEX resin is contacted with a nuclease wash buffer; and wherein (iii) after the sample is contacted with the MMC resin, the MMC resin is contacted with a nuclease wash buffer.

In some aspects, the method comprises (i) CEX-AEX-MMC; (ii) CEX-MMC-AEX; (iii) AEX-CEX-MMC; (iv) AEX-MMC-CEX; (v) MMC-CEX-AEX; or (vi) MMC-AEX-CEX. In other aspects, the method comprises CEX-AEX-MMC. In other aspects, the method comprises AEX-CEX-MMC.

For each chromatography (e.g., CEX, AEX, and MMC), various buffers (loading buffer, elution buffer, wash buffer, etc.) and conditions can be used to maximize the yield while removing the impurities as much as possible. In some aspects, each of the chromatography comprises a loading buffer, an elution buffer, and/or a wash buffer. In some aspects, the loading buffer and the elution buffer can be the same. In other aspects, the elution buffer and the wash buffer can be the same. In other aspects, the loading and wash buffers can be the same. In some aspects, the loading and wash buffers can be the same, but the elution buffer is different from the loading and wash buffers. In other aspects, the loading buffer, the elution buffer, and the wash buffer are the same.

In some aspects, CEX elution conditions can be designed to be the same as the AEX load conditions enabling straight through operation. In some aspects, CEX elution conditions can be designed to be the same as the AEX load conditions enabling straight through operation while the CEX loading conditions (e.g., a lower pH than the elution buffer) are different from the CEX elution conditions. In some aspects, AEX elution conditions can be designed to be the same as the MMC load conditions enabling straight through operation. Straight through processing can also be accomplished by integrated dilution or in-line titration of an elution and/or a load. In some aspects, CEX and AEX columns can be duplexed (placed inline in series) to enable operation of both columns in a single unit operation; the CEX column operated in flow-through or weak partitioning mode with the flow-through directly binding to the downstream AEX column. In some aspects, the product can be eluted from the AEX with a separate elution. In some aspects, to prevent fouling and maximize reuse of the downstream column, the two columns can be separated for strips and/or other phases.

In some aspects, selective loading, capture, elution, and/or wash can be achieved by changing salt, phosphate, or calcium concentrations, changing pH, altering temperature, adding organic modifiers, organic solvents, small molecules, detergents, zwitterions, amino acids, polymers, polyols (sucrose, glucose, trehalose, mannose, sorbitol, mannitol, glycerol, etc.), anti-oxidants (e.g., methionine), EDTA, EGTA, Polysorbate 20, Polysorbate 80, ethylene glycol, propylene glycol, polyethylene glycol, polypropylene glycol, and/or urea, adding excipients that alter the surface tension of the solution, adding excipients that alter the polarity of the solution, altering the residence time to take advantage of differential desorption rates between impurities and EVs, adding excipients that modulate the structure of the EVs, or any combination of the above.

In some aspects, loading, capture, elution, and/or wash can be achieved by using EDTA to inhibit any potential contaminating metalloproteases. In some aspects, the EDTA is present at a concentration of from about 0.0001M to about 1M in a buffer, e.g., an elution buffer for the AEX. In some aspects, the EDTA is present at a concentration of from about 0.001M to about 1M. In some aspects, the EDTA is present at a concentration of from about 0.001M to about 0.1M, from about 0.001M to about 0.09M, from about 0.002M to about 0.08M, from about 0.003M to about 0.07M, from about 0.004M to about 0.06M, from about 0.005M to about 0.05M, from about 0.006M to about 0.04M, from about 0.007M to about 0.03M, from about 0.008M to about 0.02M, or from about 0.009M to about 0.01M. In some aspects, the EDTA is present at a concentration of from about 0.01M to about 0.1M. In some aspects, the EDTA is present at a concentration of about 0.001M. In some aspects, the EDTA is present at a concentration of about 0.001M. In some aspects, the EDTA is present at a concentration of about 0.005M. In some aspects, the EDTA is present at a concentration of about 0.01M. In some aspects, the EDTA is present at a concentration of about 0.02M. In some aspects, the EDTA is present at a concentration of about 0.003M. In some aspects, the EDTA is present at a concentration of about 0.004M. In some aspects, the EDTA is present at a concentration of about 0.05M. In some aspects, the EDTA is present at a concentration of about 0.06M. In some aspects, the EDTA is present at a concentration of about 0.07M. In some aspects, the EDTA is present at a concentration of about 0.08M In some aspects, the EDTA is present at a concentration of about 0.09M In some aspects, the EDTA is present at a concentration of about 0.1M.

In some aspects, selective loading, elution, and/or wash of EVs can be achieved by increasing the concentration of a monovalent salt (e.g., sodium chloride, potassium chloride, sodium bromide, lithium chloride, sodium iodide, potassium bromide, lithium bromide, sodium fluoride, potassium fluoride, lithium fluoride, lithium iodide, sodium acetate, potassium acetate, lithium acetate, and potassium iodide), a divalent or trivalent salt (e.g., calcium chloride, magnesium chloride, calcium sulfate, sodium sulfate, magnesium sulfate, chromium trichloride, chromium sulfate, sodium citrate, iron (III) chloride, yttrium (III) chloride, potassium phosphate, potassium sulfate, sodium phosphate, ferrous chloride, calcium citrate, magnesium phosphate, and ferric chloride), or a combination thereof, in the elution buffer for a chromatography (e.g., CEX, AEX, and/or MMC), through the use of an increasing gradient (step or linear) of a monovalent salt (e.g., sodium chloride, potassium chloride, sodium bromide, lithium chloride, sodium iodide, potassium bromide, lithium bromide, sodium fluoride, potassium fluoride, lithium fluoride, lithium iodide, sodium acetate, potassium acetate, lithium acetate, and potassium iodide), a divalent or trivalent salt (e.g., calcium chloride, magnesium chloride, calcium sulfate, sodium sulfate, magnesium sulfate, chromium trichloride, chromium sulfate, sodium citrate, iron (III) chloride, yttrium (III) chloride, potassium phosphate, potassium sulfate, sodium phosphate, ferrous chloride, calcium citrate, magnesium phosphate, and ferric chloride), or a combination thereof, at a fixed pH. In some aspects, one or more buffers, e.g., elution buffer or loading buffer, e.g., elution buffer for AEX, loading buffer for AEX, comprises NaCl.

In some aspects, substantial EV purity can be achieved by flowing through impurities during the column loading phase, eluting impurities during selective excipient washes, and/or by selectively eluting a target during elution while leaving additional impurities bound to the column. Absorbance measurements of column eluates can suggest changes (e.g., a significant reduction) in concentrations of proteins and nucleic acids. In some aspects, the interaction between the chromatographic resins (e.g., CEX, AEX, and/or MMC) and EVs is sufficient to enable direct capture from cell culture, clarified cell culture, concentrated cell culture, or partially purified in-process pools.

In some aspects, excipients can be used for the washing step for one or more chromatography processes (e.g., CEX, AEX, and/or MMC). Excipient washes can improve purity or further aid in enriching, depleting, or isolating sub-populations of EVs. In some aspects, the excipient can be a solution having specific pH ranges, salts, organic solvents, small molecules, detergents, zwitterions, amino acids, polymers, and any combination of the above.

In some aspects, the excipient can comprise arginine, lysine, glycine, histidine, calcium, sodium, lithium, potassium, iodide, magnesium, iron, zinc, manganese, urea, propylene glycol, aluminum, ammonium, guanidinium polyethylene glycol, EDTA, EGTA, a detergent, chloride, sulfate, carboxylic acids, sialic acids, phosphate, acetate, glycine, borate, formate, perchlorate, bromine, nitrate, dithiothreitol, beta mercaptoethanol, or tri-n-butyl phosphate.

In some aspects, the excipient can also comprise a detergent. In some aspects, the detergent is selected from cetyl trimethylammonium chloride, octoxynol-9, TRITON™ X-100 (i.e., polyethylene glycol p-(1,1,3,3-tetramethylbutyl)-phenyl ether) and TRITON™ CG-110 available from Sigma-Aldrich; sodium dodecyl sulfate; sodium lauryl sulfate; deoxycholic acid; Polysorbate 80 (i.e., Polyoxyethylene (20) sorbitan monooleate); Polysorbate 20 (i.e., Polyoxyethylene (20) sorbitan monolaurate); alcohol ethoxylate; alkyl polyethylene glycol ether; decyl glucoside; octoglucosides; SafeCare; ECOSURF™ EH9, ECOSURF™ EH6, ECOSURF™ EH3, ECOSURF™ SA7, and ECOSURF™ SA9 available from DOW Chemical; LUTENSOL™ M5, LUTENSOL™ XL, LUTENSOL™ XP and APG™ 325N available from BASF; TOMADOL™ 900 available from AIR PRODUCTS; NATSURF™ 265 available from CRODA; SAFECARE™1000 available from Bestchem, TERGITOL™ L64 available from DOW; caprylic acid; CHEMBETAINE™ LEC available from Lubrizol; Mackol DG, and mixtures thereof.

In some aspects of the multistep process, any unit operation (i.e., any step in the process) can be run in batch, semi-batch, semi-continuous, or continuous mode. In some aspects, surge tanks can be employed to enable semi-continuous or continuous processing.

In other aspects, the sequence of the chromatography process (e.g., CEX-AEX-MMC or AEX-CEX-MMC) can be repeated at least two times, at least three times, at least four times, at least five times, at least six times, at least seven times, at least eight times, at least nine times, at least ten times, at least 11 times, at least 12 times, at least 13 times, at least 14 times, at least 15 times, at least 16 times, at least 17 times, at least 18 times, at least 19 times, or at least 20 times.

In some aspects, AEX and MMC columns are duplexed (placed inline in series) to enable operation of both columns in a single until operation; the AEX column is operated in bind/elute mode with the elution loaded directly onto the MMC column operation in flow-through or weak partitioning mode. In some aspects, to prevent fouling and maximize reuse of the downstream column, the two columns can be separated for strips or other phases.

In some aspects, the methods of the present disclosure comprises two or more processes (e.g., chromatographies) connected for continuous manufacturing (e.g., purification). In some aspects, the continuous manufacturing (e.g., purification) processes are integrated with the bioreactor that produces the EVs.

II.B.1. CEX Chromatography Resins

The CEX process is a form of ion exchange chromatography that separates samples based on their net surface charge. CEX specifically uses negatively charged ligands having affinity to targets having positive surface charges. Without being bound by a particular theory, EVs may be amphoteric and present positive surface charges that can be exploited for CEX purification under certain purification conditions. The method can rely on positive charges of the surface proteins on the EVs that contain basic amino acids such as lysine and arginine and/or are complexed with bivalent positively charged metals. In addition, the presence of chromatin can offer an array of basic histone proteins for CEX binding.

Various CEX resins can be used in the CEX process. In some aspects, CEX resins comprise a CEX ligand and a base matrix. In some aspects, the base matrix can comprise membranes, monoliths, hydrogels, nanofiber, composite resins, beaded resins, beaded resins with inert porous shells, an/or any other absorptive or convective media. In other aspects the base matrix can comprise materials such as cellulose, agarose, polystyrene derivatives, polyvinyl ether, silica, methacrylate derivatives, glass, ceramic hydroxyapatite, acrylamide, other backbones commonly used in chromatography and known by those of skill in the art, and/or mixtures thereof.

Various CEX ligands can be used in the CEX process. In some aspects, the CEX ligands comprise sulfate, sulfopropyl, sulfobutyl, sulfoisobutyl, sulfoethyl, sulfonate, sulfonic acid, carboxymethyl, carboxylic acid, glutamic acid, aspartic acid, histidine, hydroxyl, and/or phosphate ligands. In some aspects, CEX ligands are used together with other conventional chromatography ligands such as sulfate ligands, tertiary amine ligands, quaternary amine ligands, diethaminoethyl ligands, butyl ligands, hexyl ligands, ether ligands, polypropylene glycol ligands, phenyl ligands, ceramic hydroxy apatite ceramic fluoroapatite ligands, amino acid ligands, or any combination thereof. In some aspects, commercially available chromatography ligands are used, for example, those formulated as SP SEPHAROSE™ FF, SP SEPHAROSE™ HP, SP SEPHAROSE™ BB, SP SEPHAROSE™ XL, CM SEPHAROSE™ FF, CM SEPHAROSE™ HP, SOURCE™ 15S, SOURCE™ 30S, CAPTO™ S, MacroCap SP, CAPTO™ SP ImpRes, or CAPTO™ S ImpAct available from GE Healthcare; FRACTOGEL® EMD SO3− (M), FRACTOGEL® EMD SO3− (S), FRACTOGEL® EMD SE Hicap (M), ESHMUNO® S, or ESHMUNO® CPX available from Merck Millipore; TOYOPEARL® CM-650C, TOYOPEARL® CM-650M, TOYOPEARL® CM-650S, TOYOPEARL® SP-650C, TOYOPEARL® SP-650M, TOYOPEARL® SP-650S, TOYOPEARL® SP-550C, TOYOPEARL® MEGACAP® II SP-550 EC, TOYOPEARL® GIGACAP® S-650M, TOYOPEARL® GIGACAP® CM-650M, or TOYOPEARL® GIGACAP® S-650S available from Tosoh Bioscience; MACRO-PREP® High S, MACRO-PREP® 25 S, MACRO-PREP® CM, UNOSPHERE™ S, NUVIA™ S, or NUVIA™ HR-S available from BioRad Laboratories; S HYPERCEL™, CM Ceramic HYPERD® F, S Ceramic HYPERD® 20, S Ceramic HYPERD® F, CMM HYPERCEL™, or HYPERCEL™ STAR CEX, available from Pall Corporation; POROS® 50 HS, POROS® 20 HS, or POROS® XS, available from Thermo Fisher Scientific/Life Technologies; PL-SCX 1000 Å 30 μm or PL-SCX 1000 Å 10 μm, available from Agilent Technologies; CELLUFINE® MAX S-r, CELLUFINE® MAX S-h, or CELLUFINE® C-500 (m), available from INC Corporation; BAKERBOND™ POLYABx or BAKERBOND™ POLYABx, available from Avantor Pharmaceutical Materials; YMC—BioPro 530, YMC—BioPro S75, YMC—BioPro SmartSep S10, YMC—BioPro SmartSep 530, or YMC—BioPro SmartSep 530, available from YMC; or PRAESTO™ SP45, PRAESTO™ SP65, or PRAESTO™ SP65, available from Purolite. In some aspects, the CEX resin used in the purification process can be POROS® XS, available from Thermo Fisher Scientific/Life Technologies. In some aspects, a CEX ligand for the CEX process is POROS® XS. In some aspects, a CEX ligand for the CEX process is CMM HyperCel™.

Interactions between the ligands and EVs are influenced by several factors, such as cation exchangers, flow rate, particle size of the resin, binding capacity, or any combination thereof. In certain aspects the present disclosure further provides conditions where EVs can be effectively isolated, purified or sub-fractionated with cation exchange ligands. In some aspects, the binding of EVs to CEX ligands is strengthened in lower pH. In some aspects, the pH of the CEX loading buffer is from about 5.0 to about 7.0.

In some aspects, the binding of EVs to CEX ligands is strengthened in lower salt concentrations. In some aspects, the CEX loading buffer comprises a salt concentration from about 10 mM to about 300 mM, from about 20 mM to about 300 mM, from about 30 mM to about 250 mM, from about 40 mM to about 200 mM, from about 50 mM to about 150 mM, from about 60 mM to about 150 mM, from about 70 mM to about 150 mM, from about 80 mM to about 150 mM, from about 90 mM to about 150 mM, from about 100 mM to about 150 mM, from about 110 mM to about 150 mM, or from about 120 mM to about 150 mM. In other aspects, the CEX loading buffer comprises a salt concentration of about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, or about 200 mM. In some aspects, the salt concentration of the CEX loading buffer is about 130 mM, about 135 mM, about 137 mM, or about 140 mM.

In some aspects, CEX is performed in a bind-elute mode. In some aspects, CEX is performed in a flow-through mode. In some aspects, CEX is performed in a weak-partitioning mode, where the EVs are bound more weakly that impurities which bind more strongly to the CEX resin.

In the weak-partitioning mode, at least some desired EVs and at least some undesired EVs or impurities, both bind to the chromatographic medium. However, undesired EVs or impurities bind more tightly to the medium. Unbound, desired EVs pass through the medium and are recovered from the column effluent. The binding between EVs and the chromatographic medium is intermediate in comparison to bind-elute and flow-through modes.

In some aspects, a loading phase can be followed by a wash phase to increase recovery of the desired product. Washing can be done with a washing buffer identical to or different from the loading buffer. When different, the wash buffer is different from the loading buffer in terms of composition or pH.

In some aspects, the pH of the CEX wash buffer is higher than the pH of the CEX loading buffer.

In some aspects, the CEX wash buffer comprises a salt concentration from about 300 mM to about 5M, from about 300 mM to about 4M, from about 300 mM to about 3M, from about 400 mM to about 3M, from about 500 mM to about 3M, from about 600 mM to about 2.5M, from about 700 mM to about 2.5M, from about 800 mM to about 2.5M, from about 900 mM to about 2.5M, from about 1M to about 2.4M, from about 1M to about 2.3M, or from about 1.5M to about 2M. In other aspects, the CEX wash buffer comprises a salt concentration of about 300 mM, about 400 mM, about 500 mM, about 600 mM, about 700 mM, about 800 mM, about 900 mM, about 1M, about 1.1M, about 1.2M, about 1.3M, about 1.4M, about 1.5M, about 1.6M, about 1.7M, about 1.8M, about 1.9M, about 2.0M, about 2.1M, about 2.2M, about 2.3M, about 2.4M, about 2.5M, about 2.6M, about 2.7M, about 2.8M, about 2.9M, or about 3.0M. In some aspects, the salt concentration of the CEX wash buffer is about 1M, about 1.5M, about 2.0M, or about 2.5M. In some aspects, the salt concentration of the CEX wash buffer is about 2M.

In certain aspects, various weak-partitioning purification methods, well-known in the art, can be combined with the methods disclosed in this application. For example, in some aspects, methods for identifying ideal conditions for the weak-partitioning mode or purification methods disclosed in the U.S. Publication No. 2007/0060741, which is incorporated by reference in its entirety herein, can be used.

In certain aspects the CEX process is repeated multiple times. In some aspects, the CEX process is repeated at least two times, at least three times, at least four times, at least five times, at least six times, at least seven times, at least eight times, at least nine times, at least ten times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times, at least 28 times, at least 29 times, at least 30 times, at least 31 times, at least 32 times, at least 33 times, at least 34 times, at least 35 times, at least 36 times, at least 37 times, at least 38 times, at least 39 times, at least 40 times, at least 41 times, at least 42 times, at least 43 times, at least 44 times, at least 45 times, at least 46 times, at least 47 times, at least 48 times, at least 49 times, at least 50 times. In some aspects, the CEX process is repeated at least three times. In some aspects, the CEX process is repeated at least four times. In some aspects, the CEX process is repeated at least five times. In some aspects, the CEX process is repeated at least six times.

In certain aspects, the chromatography resin comprises a CEX resin, and the nuclease wash buffer comprises at least about 0.001 M to at least about 1.0 M of the cation, e.g., Mg²⁺ or Ca²⁺. In some aspects, the chromatography resin comprises a CEX resin, and the nuclease wash buffer comprises at least about 0.05 M of the cation, e.g., Mg²⁺ or Ca²⁺. In some aspects, the chromatography resin comprises a CEX resin, and the nuclease wash buffer comprises at least about 0.1 M of the cation, e.g., Mg²⁺ or Ca²⁺. In some aspects, the chromatography resin comprises a CEX resin, and the nuclease wash buffer comprises at least about 0.15 M of the cation, e.g., Mg²⁺ or Ca²⁺. In some aspects, the chromatography resin comprises a CEX resin, and the nuclease wash buffer comprises at least about 0.2 M of the cation, e.g., Mg²⁺ or Ca²⁺. In some aspects, the chromatography resin comprises a CEX resin, and the nuclease wash buffer comprises at least about 0.25 M of the cation, e.g., Mg²⁺ or Ca²⁺. In some aspects, the chromatography resin comprises a CEX resin, and the nuclease wash buffer comprises at least about 0.3 M of the cation, e.g., Mg²⁺ or Ca²⁺. In some aspects, the chromatography resin comprises a CEX resin, and the nuclease wash buffer comprises at least about 0.35 M of the cation, e.g., Mg²⁺ or Ca²⁺. In some aspects, the chromatography resin comprises a CEX resin, and the nuclease wash buffer comprises at least about 0.4 M of the cation, e.g., Mg²⁺ or Ca²⁺. In some aspects, the chromatography resin comprises a CEX resin, and the nuclease wash buffer comprises at least about 0.45 M of the cation, e.g., Mg²⁺ or Ca²⁺. In some aspects, the chromatography resin comprises a CEX resin, and the nuclease wash buffer comprises at least about 0.5 M of the cation, e.g., Mg²⁺ or Ca²⁺. In some aspects, the chromatography resin comprises a CEX resin, and the nuclease wash buffer comprises at least about 0.6 M of the cation, e.g., Mg²⁺ or Ca²⁺. In some aspects, the chromatography resin comprises a CEX resin, and the nuclease wash buffer comprises at least about 0.7 M of the cation, e.g., Mg²⁺ or Ca²⁺. In some aspects, the chromatography resin comprises a CEX resin, and the nuclease wash buffer comprises at least about 0.8 M of the cation, e.g., Mg²⁺ or Ca²⁺. In some aspects, the chromatography resin comprises a CEX resin, and the nuclease wash buffer comprises at least about 0.9 M of the cation, e.g., Mg²⁺ or Ca²⁺. In some aspects, the chromatography resin comprises a CEX resin, and the nuclease wash buffer comprises at least about 1.0 M of the cation, e.g., Mg²⁺ or Ca²⁺.

II.B.2. Anion Exchange Chromatography (AEX) Chromatography Resins

In certain aspects of the present disclosure, the chromatography resin comprises an AEX chromatography resin. AEX is another form of ion exchange chromatography that separates samples based on their surface charge. AEX uses positively charged ligands having affinity to targets having negative surface charges. In some aspects, the AEX can be performed on the sample comprising EVs after the sample has been subjected to a CEX. In other aspects, the AEX can be performed on the sample comprising EVs before the sample has been subjected to a CEX. In some aspects, the AEX can be performed on the sample comprising EVs before the sample has been subjected to an MMC. In some aspects, the AEX can be performed on the sample comprising EVs after the sample has been subjected to an MMC.

In some aspects, AEX is performed in a weak-partitioning mode. In some aspects, AEX is performed in flow-through mode. In some aspects, AEX is performed in a bind-elute mode.

In bind-elute mode, desired EVs bind to chromatographic medium and are eluted from the medium by elution buffers. These methods generally comprise the steps of applying or loading a sample comprising EVs, optionally washing away unbound sample components using appropriate buffers that maintain the binding interaction between EVs and affinity ligands and eluting (dissociating and recovering) EVs from the immobilized ligands by altering buffer conditions so that the binding interaction no longer occurs.

In some aspects, exchange resin can be eluted with a particular elution buffer and selected fractions of the eluate can be concentrated (e.g., by dialysis) to provide an enriched EV preparation. In certain aspects, the AEX resin used in the scalable method is of a sufficient size to accommodate large scale volumes of conditioned culture media. In other aspects, a second elution of the collected fractions from a first passage over an anion exchange column can be performed. In some aspects, the AEX is repeated at least two times, at least three times, at least four times, at least five times, at least six times, at least seven times, at least eight times, at least nine times, at least ten times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times, at least 28 times, at least 29 times, at least 30 times, at least 31 times, at least 32 times, at least 33 times, at least 34 times, at least 35 times, at least 36 times, at least 37 times, at least 38 times, at least 39 times, at least 40 times, at least 41 times, at least 42 times, at least 43 times, at least 44 times, at least 45 times, at least 46 times, at least 47 times, at least 48 times, at least 49 times, at least 50 times. In some aspects, the AEX is repeated at least three times. In some aspects, the AEX is repeated at least four times. In some aspects, the AEX is repeated at least five times. In some aspects, the AEX is repeated at least six times.

AEX resin refers to a solid phase which is positively charged, e.g. having one or more positively charged ligands. In some aspects, the ligands are selected from diethylaminopropyl, diethylaminoethyl, quaternary aminoethyl, quaternary ammonium, carboxymethyl, carboxylic acid, glutamic acid, aspartic acid, histidine, hydroxyl, phosphate, tertiary amines, quaternary amines, diethaminoethyl, dimethylaminoethyl, trimethylaminoethyl, an amino acid ligand, or combinations thereof. Commercially available anion exchange resins include DEAE cellulose, QAE SEPHADEX and FAST Q SEPHAROSE (Pharmacia). In certain aspects the chromatography ligands can be bound to a base matrix. In some aspects, the base matrix can comprise monoliths, hydrogels, porous devices, nanofibers, composite resins, beaded resins, beaded resin with inert porous shells, and/or any other solid or porous support. In some aspects, the base matrix can comprise cellulose, agarose, polystyrene derivatives, polyvinyl ether, silica, methacrylate derivatives, glass, ceramic hydroxyapatite, acrylamide, and/or other backbones commonly used in chromatography.

Examples of anion exchange resins include, but are not limited to: Q SEPHAROSE™ FF, Q SEPHAROSE™ HP, Q SEPHAROSE™ BB, Q SEPHAROSE™ XL DEAE SEPHAROSE™ FF, ANX SEPHAROSE™ 4FF low sub, ANX SEPHAROSE™ 4FF high sub, SOURCE™ 15Q, SOURCE™ 30Q, CAPTO™ Q, CAPTO™ DEAE, or CAPTO™ Q ImpRes, available from GE Healthcare; FRACTOGEL® EMD DEAE (M), FRACTOGEL® EMD TMAE (M), FRACTOGEL® EMD TMAE (S), FRACTOGEL® EMD TMAE Hicap (M), FRACTOGEL® EMD TMAE Medcap (M), ESHMUNO® Q or ESHMUNO® Q, available from Merck Millipore; TOYOPEARL® DEAE-650C, TOYOPEARL® DEAE-650M, TOYOPEARL® DEAE-650S, TOYOPEARL® SuperQ-650C, TOYOPEARL® SuperQ-650M, TOYOPEARL® SuperQ-650S, TOYOPEARL® QAE-550C, TOYOPEARL® GIGACAP® Q-650M, TOYOPEARL® Q-600C AR, TOYOPEARL® GIGACAP® DEAE-650M, TOYOPEARL® GIGACAP® Q-650S, TOYOPEARL® NH2-750F, TSKGEL® SuperQ-5PW (20 μm), or TSKGEL® SuperQ-5PW (30 μm), available from Tosoh Bioscience; MACRO-PREP® DEAE, MACRO-PREP® High Q, MACRO-PREP® 25 Q, UNOSPHERE™ Q or NUVIA™ Q, available from BioRad Laboratories; Q HYPERCEL™, DEAE Ceramic HYPERD® F, Q Ceramic HYPERD® 20, Q Ceramic HYPERD® F, or HYPERCEL™ STAR AX, available from Pall Corporation; POROS® 50 HQ, POROS® 50 PI, POROS® 50 D, POROS® 20 HQ, or POROS® XQ, available from Thermo Fisher Scientific/Life Technologies; DEAE PuraBead HF, available from Prometic Bioseparations; PL-SAX 1000 Å 30 μm, or PL-SAX 1000 Å 10 μm, available from Agilent Technologies; CELLUFINE® MAX Q-h, or CELLUFINE® Q-500 (m), available from INC Corporation; BAKERBOND™ POLYQUAT, BAKERBOND™ POLYPEI, or BAKERBOND™ POLYPEI, available from Avantor Pharmaceutical Materials; YMC—BioPro Q30, YMC—BioPro Q75, YMC—BioPro SmartSep Q10, or YMC—BioPro SmartSep Q30, available from YMC; Sartobind Q, available from 8 mm; or PRAESTO™ Q65 or PRAESTO™ Q90, available from Purolite. In some aspects the AEX resin can be Sartobind Q, available from 8 mm. In some aspects, an AEX resin for the AEX process is SARTOBIND® Q (8 mm).

In some aspects, binding of EVs to AEX ligands is strengthened in higher pH compared to the CEX process as described herein. In other aspects, binding of EVs to AEX ligands is strengthened in lower salt conditions compared to one or more chromatography processes, (e.g., CEX and/or MMC). Accordingly, the methods can further comprise the step of changing (raising or lowering) the salt concentration or pH of the sample before loading the sample to the AEX resin. In some aspects, the pH and the salt concentration for the AEX process are selected for inducing precipitation of contaminant proteins. In some aspects, the AEX chromatography is conducted at a pH from about 7 to about 10. In some aspects, the pH of the AEX loading buffer is about 7.4.

In some aspects, the AEX loading buffer comprises a salt concentration from about 10 mM to about 1000 mM, from about 50 mM to about 900 mM, from about 60 mM to about 800 mM, from about 70 mM to about 700 mM, from about 80 mM to about 700 mM, from about 80 mM to about 800 mM, from about 90 mM to about 700 mM, from about 100 mM to about 700 mM, from about 150 mM to about 700 mM, from about 200 mM to about 700 mM, from about 300 mM to about 600 mM, from about 400 mM to about 600 mM, from about 500 mM to about 600 mM, from about 500 mM to about 700 mM, or from about 500 mM to about 800 mM. In some aspects, the AEX loading buffer comprises a salt concentration of about 100 mM, about 150 mM, about 200 mM, about 250 mM, about 300 mM, about 350 mM, about 400 mM, about 450 mM, about 500 mM, about 550 mM, about 600 mM, about 650 mM, about 700 mM, about 800 mM, about 900 mM, or about 1M.

In some aspects the AEX elution buffer comprises a salt concentration from about 600 mM to about 1500 mM, from about 700 mM to about 1400 mM, from about 800 mM to about 1300 mM, from about 900 mM to about 1200 mM, from about 800 mM to about 1500 mM, from about 700 mM to about 1500 mM, from about 800 mM to about 1400 mM, from about 600 mM to about 1300 mM, from about 600 mM to about 1400 mM, from about 600 mM to about 1200 mM, from about 600 mM to about 1100 mM, or from about 1000 mM to about 1500 mM.

In other aspects, the AEX wash buffer comprises a salt concentration from about 1M to about 3M, from about 1M to about 2.9M, from about 1.1M to about 2.9M, from about 1.5M to about 2.5M, from about 1.6M to about 2.4M, from about 1.7M to about 2.3M, from about 1.8M to about 2.2M, or from about 1.9M to about 2.1M. In other aspects, the AEX wash buffer comprises a salt concentration about 1M, about 1.1M, about 1.2M, about 1.3M, about 1.4M, about 1.5M, about 1.6M, about 1.7M, about 1.8M, about 1.9M, about 2.0M, about 2.1M, about 2.2M, about 2.3M, about 2.4M, about 2.5M, about 2.6M, about 2.7M, about 2.8M, about 2.9M, or 3.0M. In some aspects, the AEX wash buffer comprises a salt concentration about 2M.

In certain aspects, the chromatography resin comprises an AEX resin, and the nuclease wash buffer comprises at least about 0.001 M to at least about 1.0 M of the cation, e.g., Mg²⁺ or Ca²⁺. In some aspects, the chromatography resin comprises an AEX resin, and the nuclease wash buffer comprises at least about 0.05 M of the cation, e.g., Mg²⁺ or Ca²⁺. In some aspects, the chromatography resin comprises an AEX resin, and the nuclease wash buffer comprises at least about 0.1 M of the cation, e.g., Mg²⁺ or Ca²⁺. In some aspects, the chromatography resin comprises an AEX resin, and the nuclease wash buffer comprises at least about 0.15 M of the cation, e.g., Mg²⁺ or Ca²⁺. In some aspects, the chromatography resin comprises an AEX resin, and the nuclease wash buffer comprises at least about 0.2 M of the cation, e.g., Mg²⁺ or Ca²⁺. In some aspects, the chromatography resin comprises an AEX resin, and the nuclease wash buffer comprises at least about 0.25 M of the cation, e.g., Mg²⁺ or Ca²⁺. In some aspects, the chromatography resin comprises an AEX resin, and the nuclease wash buffer comprises at least about 0.3 M of the cation, e.g., Mg²⁺ or Ca²⁺. In some aspects, the chromatography resin comprises an AEX resin, and the nuclease wash buffer comprises at least about 0.35 M of the cation, e.g., Mg²⁺ or Ca²⁺. In some aspects, the chromatography resin comprises an AEX resin, and the nuclease wash buffer comprises at least about 0.4 M of the cation, e.g., Mg²⁺ or Ca²⁺. In some aspects, the chromatography resin comprises an AEX resin, and the nuclease wash buffer comprises at least about 0.45 M of the cation, e.g., Mg²⁺ or Ca²⁺. In some aspects, the chromatography resin comprises an AEX resin, and the nuclease wash buffer comprises at least about 0.5 M of the cation, e.g., Mg²⁺ or Ca²⁺. In some aspects, the chromatography resin comprises an AEX resin, and the nuclease wash buffer comprises at least about 0.6 M of the cation, e.g., Mg⁺ or Ca²⁺. In some aspects, the chromatography resin comprises an AEX resin, and the nuclease wash buffer comprises at least about 0.7 M of the cation, e.g., Mg²⁺ or Ca²⁺. In some aspects, the chromatography resin comprises an AEX resin, and the nuclease wash buffer comprises at least about 0.8 M of the cation, e.g., Mg²⁺ or Ca²⁺. In some aspects, the chromatography resin comprises an AEX resin, and the nuclease wash buffer comprises at least about 0.9 M of the cation, e.g., Mg²⁺ or Ca²⁺. In some aspects, the chromatography resin comprises an AEX resin, and the nuclease wash buffer comprises at least about 1.0 M of the cation, e.g., Mg²⁺ or Ca²⁺.

II.B.3. Multi-modal Chromatography (MMC)

In some aspects, the chromatography resin comprises a mixed mode chromatography (“MMC”) resin. In some aspects, samples comprising EVs are purified by MMC after being purified by AEX. In some aspects, samples comprising EVs are purified by MMC before being purified by AEX. In some aspects, samples comprising EVs are purified by MMC after being purified by CEX. In some aspects, samples comprising EVs are purified by MMC before being purified by CEX. In some aspects, samples purified by AEX or CEX are processed by depth filtration before further being processed by MMC. In some aspects, adsorptive depth filter is used. In some aspects, an AEX-processed sample further processed by depth filtration is applied to MMC for purification.

Mixed mode chromatography employs chromatographic resins containing ligands possessing more than one type of functional groups. This unique property of mixed mode resin enables binding through multiple chromatographic modes in a single resin. Most resins in this class comprise a ligand containing a hydrophobic group (e.g. phenyl, benzyl, propyl, butyl, etc.) and a charged group (e.g. cation: sulfate, carboxylic acid, methyl carboxylic acid; or an anion: quaternary amine, diethylaminoethyl, diethylaminopropyl, or quaternary ammonium). However, some resins may also contain a hydrophilic group in place of the hydrophobic group, (e.g. silica, urea, polyethyleneimine, amino or amide groups, cyanopropyl, diol, or aminopropyl).

In some aspects, MMC resins comprises conventional chromatography ligands. In some aspects, the ligands are selected from tertiary amines, quaternary amines, diethaminoethyl, ceramic hydroxyapatite, ceramic fluoroapatite, butyl, hexyl, ether, hydroxyl, polypropylene glycol, phenyl, benzyl, sulfate, sulfopropyl, sulfobutyl, sulfoisobutyl, sulfoethyl, sulfonate, sulfonic acid, carboxymethyl, carboxylic acid, glutamic acid, aspartic acid, histidine, hydroxyl, phosphate ligands, and mixtures thereof. In some aspects, the chromatography ligands are formulated as CAPTO™ MMC, CAPTO™ adhere, CAPTO™ MMC ImpRes, CAPTO™ adhere ImpRes, CAPTO™ Core 700, or CAPTO™ Core 700, available from GE Healthcare; ESHMUNO® HCX, available from Merck Millapore; TOYOPEARL® MX-Trp-650M, available from Tosoh Bioscience; NUVIA™ CPRIME™, available from BioRad Laboratories; or CMM HYPERCEL™, HEA HYPERCEL™ or PPA HyperCel™, available from Pall Corporation; In some aspects, the MMC resin is a resin used in other types of chromatography (i.e., AEX, CEX, HIC, HCIC, etc.). In some aspects, the MMC resin is CMM HYPERCEL™, available from Pall Corporation.

In some aspects, the resins used in MMC comprise anion-exchange/reversed-phase (AEX/RP), cation-exchange/reversed phase (CEX/RP), anion-exchange/cation-exchange/reversed phase (AEX/CEX/RP), AEX/hydrophilic (AEX/HILIC), CEX-hydrophilic (CEX/HILIC), or AEX/CEX hydrophilic (AEX/CEX/HILIC). An example of AEX/RP ligand is a hydrophobic, anionic ligand with hydrogen bonding that contains a quaternary amine, a phenyl group, and allows hydrogen bonding. An example of a CEX/RP ligand is a cationic ligand with hydrophobic binding that contain a secondary amine and is cationic over a wide pH range. Some mixed mode ligands are pH controllable, such as those containing 4-mercaptoethylpyridine ligands. The uncharged nitrogen in the pyridine ring becomes charged as pH decreases, resulting in a pH controllable mixed-mode ligand.

In some aspects, mixed mode ligands can be immobilized on the base matrix. In some aspects, the base matrix comprises membranes, monoliths, beaded resins, nanofibers, and/or other absorptive or convective media. In some aspects, the base matrix comprises cellulose, agarose, polystyrene derivatives, silica, methacrylate derivatives, glass, ceramic hydroxyapatite, PVDF, PTFE, polyethersulfone, polypropylene, polyethylene, acrylamide, and/or any mixtures or derivatives thereof.

Mixed mode media comprising a single or plurality of ligands and a base matrix can be classified into four categories based on the arrangement of the ligand substrates on the base matrix. Type I media are mixtures of separation media, each with a single chemistry, packed to form a column. Type II media comprise substrates modified with a mixture of ligands having different functionalities, such as ion exchange, reverse phase, or hydrophilic phase properties. In Type III media, the functional ligands can be “embedded” in a hydrophobic chain, or in Type IV media, the hydrophobic chain can be “tipped” with the functional group. The mixed mode resins comprising a base matrix and one or more functional groups may be comprised of any of the types of media as described herein.

In some aspects, a MMC chromatography column is generated with the resin disclosed herein. The resin can be formed in a suspension, in slurry, or can be packed into a chromatography column.

In some aspects, the MMC chromatography column can further comprise conventional chromatography ligands selected from sulfate, tertiary amines, quaternary amines, carboxy methyl, carboxylic acids, diethaminoethyl, ceramic hydroxy apatite and ceramic fluoroapatite, or any combination thereof. In some aspects, conventional chromatography ligands can be formulated as CAPTO MMC™ or CAPTO ADHERE™ available from GE Healthcare Life Sciences; TOYOPEARL MX-TRP™ available from Tosoh Bioscience; HYPERCEL™ STAR AX available from Pall Corporation; NUVIA™ CPRIME™ available from BioRad; or ESHMUNO™ HCX available from EMD Millipore.

In some aspects, hydrophobic, hydrophilic, and/or ionic mixed mode ligands and the conventional chromatography ligands are displayed on the same resin. For example, the hydrophobic, hydrophilic, and/or ionic mixed mode ligands and the conventional chromatography ligands are immobilized on the base matrix (e.g., membranes, monoliths, beaded resins, nanofibers, and other absorptive or convective media). In some aspects, hydrophobic, hydrophilic, and/or ionic mixed mode ligands and chromatographic ligands are intermixed. In some aspects, hydrophobic, hydrophilic, and/or ionic mixed mode ligands and chromatographic ligands are displayed on separate layers.

In some aspects, mixed mode media comprises hydrophobic ligands. Hydrophobic ligands can be used to purify EVs based on their interaction with a nonpolar surface on EVs, an amphiphilic phospholipid bilayer membrane with embedded transmembrane proteins or an outer bilayer surface that is associated with a variety of proteins, nucleic acids, lipids, and carbohydrates. Hydrophobic groups of the biomolecules that are sufficiently exposed to the surface allow interaction with hydrophobic ligands. In some aspects, the hydrophobic ligands can be hydrophobic alkyl or aryl groups. In some aspects, the hydrophobic alkyl or aryl groups are selected from phenyl, ethyl, methyl, pentyl, heptyl, benzyl, octyl, butyl, hexyl, ether, hydroxyl, polypropylene glycol, and the like.

In some aspects, mixed mode media comprises hydrophilic ligands. Hydrophilic ligands can be used to purify EVs via flow through mode, or to purify desired subgroups of EVs. The amphiphilic surface of the EVs may not bind to the hydrophilic ligands of the column, while polar impurities or proteins in the sample interact with the hydrophilic ligands. In some aspects, the hydrophilic ligands comprise, silica, urea, amino groups, amide groups, polyethyleneimine, cyanopropyl, diol, aminopropyl, and/or zwitterions such as sulnfoalkylbetaine.

In some aspects, mixed mode media comprises CEX ligands.

In some aspects, mixed mode media comprises AEX ligands.

In some aspects, MMC chromatography is performed in a bind-elute mode. In some aspects, MMC chromatography is performed in a weak-partitioning mode.

According to the present disclosure, additional chromatography process can be used in addition to the chromatography processes disclosed herein (e.g., CEX-AEX or CEX-AEX-MMC). In some aspects, the additional chromatography can be used instead of the MMC process. In other aspects, the additional chromatography can be used in addition to the CEX, AEX, and MMC. In some aspects, a CEX, such as a CMM HYPERCEL™ chromatography column, is operated in series with a MMC, such as a CaptoCore700™ column, operated in flow through mode. In some aspects, a CEX-MMC is operated in series in flow-through mode. In some aspects, a MMC-CEX is operated in series in flow-through mode.

In some aspects, the present method further comprises hydrophobic interaction chromatography (“HIC”). In some aspects, the present method further comprises hydrophobic charge induction chromatography (“HCIC”).

The HIC or HCIC uses hydrophobic ligands attached to a base matrix. In some aspects the base matrix comprises membranes, monoliths, beaded resins, nanofibers, and/or other absorptive or convective media. In some aspects, the base matrix comprises cellulose, agarose, polystyrene derivatives, silica, methacrylate derivatives, glass, ceramic hydroxyapatite, PVDF, PTFE, polyethersulfone, polypropylene, polyethylene, acrylamide, and/or any mixtures or derivatives thereof.

Purification of EVs by hydrophobic ligands is based on the interaction between the ligands and a nonpolar surface on EVs, an amphiphilic phospholipid bilayer membrane with embedded transmembrane proteins or an outer bilayer surface that is associated with a variety of proteins, nucleic acids, lipids, and carbohydrates. Hydrophobic groups of the biomolecules that are sufficiently exposed to the surface can interact with hydrophobic ligands.

In some aspects, hydrophobic ligands that can be used for the present invention include ligands comprising hydrophobic alkyl and/or aryl groups. In some aspects the hydrophobic alkyl or aryl group are selected from phenyl, ethyl, methyl, pentyl, heptyl, benzyl, octyl, butyl, hexyl, ether, hydroxyl, polypropylene glycol, and mixtures thereof.

In some aspects, the salt concentration of the MMC loading buffer, elution buffer, and/or wash buffer is at least about 100 mM, at least about 200 mM, at least about 300 mM, at least about 400 mM, at least about 500 mM, at least about 600 mM, at least about 700 mM, at least about 800 mM, at least about 900 mM, at least about 1M, at least about 1.1M, at least about 1.2M, at least about 1.3M, at least about 1.4M, at least about 1.5M, at least about 1.6M, at least about 1.7M, at least about 1.8M, at least about 1.9M, at least about 2.0M, at least about 2.1M, at least about 2.2M, at least about 2.3M, at least about 2.4M, or at least about 2.5M. In other aspects, the sale concentration of the MMC loading buffer is between about 10 mM and about 5M, between about 100 mM and about 5M, between about 100 mM and about 4M, between about 100 mM and about 3M, between about 200 mM and about 5M, between about 300 mM and about 4M, between about 400 mM and about 3M, between about 500 mM and about 2M, between about 1M and about 3M, between about 1 mM and about 2M, between about 800 mM and about 2M, between about 900 mM and about 2.5M, or between about 1.5M and about 2.5M. In some aspects, the salt concentration of the MMC loading buffer and wash buffer is about 1M.

In some aspects, the pH of the MMC loading buffer and/or wash buffer is about 7.5.

In certain aspects, the chromatography resin comprises an MMC resin, and the nuclease wash buffer comprises at least about 0.001 M to at least about 1.0 M of the cation, e.g., Mg²⁺ or Ca²⁺. In some aspects, the chromatography resin comprises an MMC resin, and the nuclease wash buffer comprises at least about 0.05 M of the cation, e.g., Mg²⁺ or Ca²⁺. In some aspects, the chromatography resin comprises an MMC resin, and the nuclease wash buffer comprises at least about 0.1 M of the cation, e.g., Mg²⁺ or Ca²⁺. In some aspects, the chromatography resin comprises an MMC resin, and the nuclease wash buffer comprises at least about 0.15 M of the cation, e.g., Mg²⁺ or Ca²⁺. In some aspects, the chromatography resin comprises an MMC resin, and the nuclease wash buffer comprises at least about 0.2 M of the cation, e.g., Mg²⁺ or Ca²⁺. In some aspects, the chromatography resin comprises an MMC resin, and the nuclease wash buffer comprises at least about 0.25 M of the cation, e.g., Mg²⁺ or Ca²⁺. In some aspects, the chromatography resin comprises an MMC resin, and the nuclease wash buffer comprises at least about 0.3 M of the cation, e.g., Mg²⁺ or Ca²⁺. In some aspects, the chromatography resin comprises an MMC resin, and the nuclease wash buffer comprises at least about 0.35 M of the cation, e.g., Mg²⁺ or Ca²⁺. In some aspects, the chromatography resin comprises an MMC resin, and the nuclease wash buffer comprises at least about 0.4 M of the cation, e.g., Mg²⁺ or Ca²⁺. In some aspects, the chromatography resin comprises an MMC resin, and the nuclease wash buffer comprises at least about 0.45 M of the cation, e.g., Mg⁺ or Ca²⁺. In some aspects, the chromatography resin comprises an MMC resin, and the nuclease wash buffer comprises at least about 0.5 M of the cation, e.g., Mg²⁺ or Ca²⁺. In some aspects, the chromatography resin comprises an MMC resin, and the nuclease wash buffer comprises at least about 0.6 M of the cation, e.g., Mg²⁺ or Ca²⁺. In some aspects, the chromatography resin comprises an MMC resin, and the nuclease wash buffer comprises at least about 0.7 M of the cation, e.g., Mg²⁺ or Ca²⁺. In some aspects, the chromatography resin comprises an MMC resin, and the nuclease wash buffer comprises at least about 0.8 M of the cation, e.g., Mg²⁺ or Ca²⁺. In some aspects, the chromatography resin comprises an MMC resin, and the nuclease wash buffer comprises at least about 0.9 M of the cation, e.g., Mg²⁺ or Ca²⁺. In some aspects, the chromatography resin comprises an MMC resin, and the nuclease wash buffer comprises at least about 1.0 M of the cation, e.g., Mg²⁺ or Ca²⁺.

II.B.4. Affinity Chromatography

In some aspects, the chromatography resin comprises an affinity chromatography resin. Affinity chromatography separates target molecules from non-target molecules in a mixture by utilizing highly specific binding between the affinity chromatography resin and the target molecule. In some aspects, the affinity chromatography resin interacts with the EVs, e.g., exosomes. In some aspects, the affinity chromatography resin comprises a binding moiety, wherein the binding moiety interacts with a target protein on the surface of the EV, e.g., exosome. In some aspects, the binding moiety interacts with a scaffold protein. In some aspects, the binding moiety interacts with PTGFRN. In some aspects, the binding moiety interacts with a fragment of PTGFRN. In some aspects, the binding moiety interacts with a Scaffold X protein. In some aspects, the chromatography resin comprises a pseudo affinity chromatography resin.

In certain aspects, the chromatography resin comprises an affinity chromatography resin, and the nuclease wash buffer comprises at least about 0.001 M to at least about 1.0 M of the cation, e.g., Mg²⁺ or Ca²⁺. In some aspects, the chromatography resin comprises an affinity chromatography resin, and the nuclease wash buffer comprises at least about 0.001 M of the cation, e.g., Mg²⁺ or Ca²⁺. In some aspects, the chromatography resin comprises an affinity chromatography resin, and the nuclease wash buffer comprises at least about 0.002 M of the cation, e.g., Mg²⁺ or Ca²⁺. In some aspects, the chromatography resin comprises an affinity chromatography resin, and the nuclease wash buffer comprises at least about 0.003 M of the cation, e.g., Mg²⁺ or Ca²⁺. In some aspects, the chromatography resin comprises an affinity chromatography resin, and the nuclease wash buffer comprises at least about 0.004 M of the cation, e.g., Mg²⁺ or Ca²⁺. In some aspects, the chromatography resin comprises an affinity chromatography resin, and the nuclease wash buffer comprises at least about 0.005 M of the cation, e.g., Mg²⁺ or Ca²⁺. In some aspects, the chromatography resin comprises an affinity chromatography resin, and the nuclease wash buffer comprises at least about 0.006 M of the cation, e.g., Mg²⁺ or Ca²⁺. In some aspects, the chromatography resin comprises an affinity chromatography resin, and the nuclease wash buffer comprises at least about 0.007 M of the cation, e.g., Mg²⁺ or Ca²⁺. In some aspects, the chromatography resin comprises an affinity chromatography resin, and the nuclease wash buffer comprises at least about 0.008 M of the cation, e.g., Mg²⁺ or Ca²⁺. In some aspects, the chromatography resin comprises an affinity chromatography resin, and the nuclease wash buffer comprises at least about 0.009 M of the cation, e.g., Mg²⁺ or Ca²⁺. In some aspects, the chromatography resin comprises an affinity chromatography resin, and the nuclease wash buffer comprises at least about 0.01 M of the cation, e.g., Mg²⁺ or Ca²⁺.

II.C. Buffers

Certain aspects of the present disclosure are directed to methods of preparing purified EVs, e.g., exosomes, from a sample comprising EVs, e.g., exosomes, and one or more nucleic acid molecules, comprising: (i) contacting the sample with a chromatography resin and (ii) contacting the chromatography resin with a nuclease wash buffer, wherein the (ii) washing follows the (i) contacting. Some aspects of the present disclosure are directed to methods of reducing the concentration of residual nucleic acid molecule in a sample comprising EVs, e.g., exosomes, comprising (i) contacting the sample with a chromatography resin and (ii) contacting the chromatography resin with a nuclease wash buffer, wherein the (ii) washing follows the (i) contacting. In some aspects, the sample is contacted with the chromatography resin in a loading buffer, e.g., a loading buffer comprising the sample is contacted with the chromatography resin.

The loading buffer used herein can comprise any one or more elements in addition to the EVs, e.g., exosomes, and the one or more nucleic acid molecules. In certain aspects, the loading buffer comprises a salt. In some aspects, the loading buffer comprises a salt concentration of less than about 1.2 M (e.g., less than about 1.1 M, less than about 1.0 M, less than about 0.9 M, less than about 0.8 M, less than about 0.7 M, less than about 0.6 M, less than about 0.5 M, less than about 0.4 M, less than about 0.3 M, less than about 0.2 M, less than about 0.1 M.

In some aspects, the loading buffer comprises a salt concentration from at least about 0.01 M to at least about 1.0 M. In some aspects, the salt concentration of the loading buffer is at least about 0.01 M, at least about 0.05 M, at least about 0.1 M, at least about 0.15 M, at least about 0.2 M, at least about 0.25 M, at least about 0.3 M, at least about 0.35 M, at least about 0.4 M, at least about 0.45 M, at least about 0.5 M, at least about 0.55 M, at least about 0.6 M, at least about 0.65 M, at least about 0.7 M, at least about 0.75 M, at least about 0.8 M, at least about 0.85 M, at least about 0.9 M, at least about 0.95 M, or at least about 1.0 M.

In some aspects, the salt of the loading buffer is selected from NaCl, KCl, PO₄, CaCl₂), MgCl₂, and any combination thereof. In some aspects, the salt of the loading buffer is selected from NaCl, KCl, KPO₄, NaPO₄, CaCl₂), Mg₂SO₄, ZnCl₂, MnCl₂, MnSO₄, NaSCN, KSCN, LiCl, and any combination thereof. In certain aspects, the loading buffer comprises NaCl. In certain aspects, the loading buffer comprises KCl. In certain aspects, the loading buffer comprises KPO₄. In certain aspects, the loading buffer comprises CaCl₂). In certain aspects, the loading buffer comprises NaPO₄. In certain aspects, the loading buffer comprises Mg₂SO₄. In certain aspects, the loading buffer comprises ZnCl₂. In certain aspects, the loading buffer comprises MnCl₂. In certain aspects, the loading buffer comprises MnSO₄. In certain aspects, the loading buffer comprises NaSCN. In certain aspects, the loading buffer comprises KSCN. In certain aspects, the loading buffer comprises LiCl. In certain aspects, the loading buffer comprises MgCl₂. In some aspects, the loading buffer comprises a salt, wherein the salt comprises an anion selected from F⁻, SO₄²⁻, HPO₄²⁻, PO₄²⁻, acetate, Cl⁻, NO₃⁻, Br⁻, ClO₃⁻, SCN⁻ or any combination thereof. In some aspects, the loading buffer comprises HEPES, BES, Bicine, MES, MOPS, PIPES, acetate, carbonate, citrate, bicarbonate buffer, aspartic acid, glutamic acid, and any combination thereof. In some aspects, the loading buffer comprises a salt, wherein the salt comprises a cation selected from NH₄⁺, K⁺, Na⁺, Li²⁺, Mg²⁺, Ca²⁺, guanidinium, and any combination thereof. In some aspects, the loading buffer comprises a buffer selected from the group consisting of a Imidazole, Tris, TAPS, BisTRIS, arginine, histidine, lysine buffer, and any combination thereof

In some aspects the loading buffer comprises at least about 0.01 M NaCl, at least about 0.05 M NaCl, at least about 0.1 M NaCl, at least about 0.15 M NaCl, at least about 0.2 M NaCl, at least about 0.25 M NaCl, at least about 0.3 M NaCl, at least about 0.35 M NaCl, at least about 0.4 M NaCl, at least about 0.45 M NaCl, at least about 0.5 M NaCl, at least about 0.55 M NaCl, at least about 0.6 M NaCl, at least about 0.65 M NaCl, at least about 0.7 M NaCl, at least about 0.75 M NaCl, at least about 0.8 M NaCl, at least about 0.85 M NaCl, at least about 0.9 M NaCl, at least about 0.95 M NaCl, at least about 1 M NaCl, at least about 1.1 M NaCl, 1.2 M NaCl, at least about 1.3 M NaCl, at least about 1.4 M NaCl, at least about 1.5 M NaCl, at least about 1.6 M NaCl, at least about 1.7 M NaCl, at least about 1.8 M NaCl, at least about 1.9 M NaCl, at least about 2 M NaCl. In certain aspects, the loading buffer comprises at least about 0.55 M NaCl.

In some aspects, the chromatography resin is contacted with a wash buffer, wherein the wash buffer does not comprise a nuclease. In some aspects, the chromatography resin is contacted with the wash buffer: (a) after (i) contacting the sample with a chromatography resin, and before (ii) contacting the chromatography resin with a nuclease wash buffer; (b) after (ii) contacting the chromatography resin with a nuclease wash buffer; or (c) both (a) and (b). In some aspects, the chromatography resin is washed with a first wash buffer, prior to contacting the chromatography resin with the nuclease wash buffer. In some aspects, the first wash buffer does not comprise a nuclease. In some aspects, the chromatography resin is washed with a second wash buffer after contacting the chromatography resin with the nuclease wash buffer.

In some aspects, the method comprises:
a. contacting the sample with a chromatography resin, wherein the EVs, e.g., exosomes associate with the chromatography resin;
b. applying a first wash buffer to the chromatography resin;
c. contacting the chromatography resin with a nuclease wash buffer;
d. applying a second wash buffer to the chromatography resin;
e. applying an elution buffer to the chromatography resin; and
f. collecting the eluent, wherein the eluent comprises the EVs, e.g., exosomes.

In some aspects, the wash buffer (e.g., the first wash buffer and/or the second wash buffer) comprises a salt at a concentration of at least about 0.01 M, at least about 0.05 M, at least about 0.1 M, at least about 0.15 M, at least about 0.2 M, at least about 0.25 M, at least about 0.3 M, at least about 0.35 M, at least about 0.4 M, at least about 0.45 M, at least about 0.5 M, at least about 0.55 M, at least about 0.6 M, at least about 0.65 M, at least about 0.7 M, at least about 0.75 M, at least about 0.8 M, at least about 0.85 M, at least about 0.9 M, at least about 0.95 M, or at least about 1.0 M. In some aspects, the salt in the wash buffer is selected from NaCl, KCl, KPO₄, NaPO₄, CaCl₂), Mg₂SO₄, ZnCl₂, MnCl₂, MnSO₄, NaSCN, KSCN, LiCl, and any combination thereof.

In some aspects, the wash buffer (e.g., the first wash buffer and/or the second wash buffer) comprises NaCl. In some aspects, the wash buffer comprises at least about 0.01 M NaCl, at least about 0.05 M NaCl, at least about 0.1 M NaCl, at least about 0.15 M NaCl, at least about 0.2 M NaCl, at least about 0.25 M NaCl, at least about 0.3 M NaCl, at least about 0.35 M NaCl, at least about 0.4 M NaCl, at least about 0.45 M NaCl, at least about 0.5 M NaCl, at least about 0.55 M NaCl, at least about 0.6 M NaCl, at least about 0.65 M NaCl, at least about 0.7 M NaCl, at least about 0.75 M NaCl, at least about 0.8 M NaCl, at least about 0.85 M NaCl, at least about 0.9 M NaCl, at least about 0.95 M NaCl, or at least about 1 M NaCl. In certain aspects, the wash buffer comprises at least about 0.55 M NaCl.

In some aspects, the methods disclosed herein further comprise (iii) eluting the EVs from the chromatography resin by contacting the chromatography resin with an elution buffer, wherein (iii) occurs after (ii) contacting the chromatography resin with a nuclease wash buffer. In some aspects, the elution buffer releases one or more EVs from the chromatography resin. In certain aspects, the method further comprises collecting an eluent after contacting the chromatography resin with the elution buffer. In some aspects, the eluent comprises one or more EVs.

In some aspects, the elution buffer comprises a salt concentration of at least about 1.0 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2.0 M, at least about 2.5 M, at least about 3.0 M, at least about 3.5 M, at least about 4.0 M, at least about 4.5 M, or at least about 5.0 M. In some aspects, the elution buffer comprises a salt concentration of at least about 1.0 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2.0 M, at least about 2.5 M, at least about 3.0 M, at least about 3.5 M, at least about 4.0 M, at least about 4.5 M, or at least about 5.0 M NaCl. In certain aspects, the elution buffer comprises at least about 1.2 M NaCl.

II.D. Additional Purification Steps

In some aspects, the sample comprising the EVs, e.g., exosomes, and the one or more nucleic acid molecule is subjected to one or more additional purification step. In some aspects, the sample is subject to one or more additional purification step prior to (i) contacting the sample with a chromatography resin, and (ii) contacting the chromatography resin with a nuclease wash buffer. In some aspects, the sample is subject to one or more additional purification step after to (i) contacting the sample with a chromatography resin, and (ii) contacting the chromatography resin with a nuclease wash buffer. In some aspects, the sample is subject to one or more additional purification step both prior to and after (i) contacting the sample with a chromatography resin, and (ii) contacting the chromatography resin with a nuclease wash buffer.

In some aspects, one or more filtration steps are added before, after, or between the chromatographic purification steps. For example, adsorptive depth filtrations step can be added before, between, or after chromatographic steps: (i) Filtration-CEX-AEX-MMC; (ii) CEX-Filtration-AEX-MMC; (iii) CEX-AEX-Filtration-MMC; (iv) CEX-AEX-MMC-filtration; (v) Filtration-CEX-MMC-AEX; (vi) CEX-Filtration-MMC-AEX; (vii) CEX-MMC-Filtration-AEX; (viii) CEX-MMC-AEX-Filtration; (ix) Filtration-AEX-CEX-MMC; (x) AEX-Filtration-CEX-MMC; (xi) AEX-CEX-Filtration-MMC; (xii) AEX-CEX-MMC-Filtration; (xiii) Filtration-AEX-MMC-CEX; (xiv) AEX-Filtration-MMC-CEX; (xv) AEX-MMC-Filtration-CEX; (xvi) AEX-MMC-CEX-Filtration; (xvii) Filtration-MMC-CEX-AEX; (xvii) MMC-Filtration-CEX-AEX; (xvii) MMC-CEX-Filtration-AEX; (xvii) MMC-CEX-AEX-Filtration; (xviii) Filtration-MMC-AEX-CEX; (xix) MMC-Filtration-AEX-CEX; (xx) MMC-AEX-Filtration-CEX; or (xxi) MMC-AEX-CEX-Filtration. Any one of filtration described herein can be used for the filtration. In some aspects, the present method comprises: (1) Filtration(1)-CEX-Filtration(2)-AEX-MMC; (2) Filtration(1)-CEX-AEX-Filtration(2)-MMC; (3) Filtration(1)-CEX-AEX-MMC-Filtration(2); (4) CEX-Filtration(1)-AEX-Filtration(2)-MMC; (5) CEX-Filtration(1)-AEX-MMC-Filtration(2); (6) CEX-AEX-Filtration(1)-MMC-Filtration(2); (7) Filtration(1)-CEX-Filtration(2)-MMC-AEX; (8) Filtration(1)-CEX-MMC-Filtration(2)-AEX; (9) Filtration(1)-CEX-MMC-AEX-Filtration(2); (10) CEX-Filtration(1)-MMC-Filtration(2)-AEX; (11) CEX-Filtration(1)-MMC-AEX-Filtration(2); (12) CEX-MMC-Filtration(1)-AEX-Filtration(2); (13) Filtration(1)-AEX-Filtration(2)-CEX-MMC; (14) Filtration(1)-AEX-CEX-Filtration(2)-MMC; (15) Filtration(1)-AEX-CEX-MMC-Filtration(2); (16) AEX-Filtration(1)-CEX-Filtration(2)-MMC; (17) AEX-Filtration(1)-CEX-MMC-Filtration(2); (18) AEX-CEX-Filtration(1)-MMC-Filtration(2); (19) Filtration(1)-AEX-Filtration(2)-MMC-CEX; (20) Filtration(1)-AEX-MMC-Filtration(2)-CEX; (21) Filtration(1)-AEX-MMC-CEX-Filtration(2); (22) AEX-Filtration(1)-MMC-Filtration(2)-CEX; (23) AEX-Filtration(1)-MMC-CEX-Filtration(2); (24) AEX-MMC-Filtration(1)-CEX-Filtration(1); (25) Filtration(1)-MMC-Filtration(2)-CEX-AEX; (26) Filtration(1)-MMC-CEX-Filtration(2)-AEX; (27) Filtration(1)-MMC-CEX-AEX-Filtration(2); (28) MMC-Filtration(1)-CEX-Filtration(2)-AEX; (29) MMC-Filtration(1)-CEX-AEX-Filtration(2); (30) MMC-CEX-Filtration(1)-AEX-Filtration(2); (31) Filtration(1)-MMC-Filtration(2)-AEX-CEX; (32) Filtration(1)-MMC-AEX-Filtration(2)-CEX; (33) Filtration(1)-MMC-AEX-CEX-Filtration(2); (34) MMC-Filtration(1)-AEX-Filtration(2)-CEX; (35) MMC-Filtration(1)-AEX-CEX-Filtration(2); or (36) MMC-AEX-Filtration(1)-CEX-Filtration(2). In some aspects, Filtration (1) is the same as Filtration (2). In other aspects, Filtration (1) is different from Filtration (2). In other aspects, any filtration prior to the CEX process has a bigger filter size compared to a filter size of filtration after the CEX process. In some aspects, the filter size of the filtrations is reduced in or after the CEX process. In some aspects, the filter size prior to the CEX process is bigger than about 0.14 micron, about 0.16 micron, about 0.18 micron, about 0.2 micron, about 0.25 micron, about 0.3 micron, about 0.35 micron, about 0.4 micron, about 0.45 micron, about 0.5 micron, about 0.55 micron, about 0.6 micron, about 0.65 micron, or about 0.7 micron. In other aspects, the filter size of the filtrations in or after the CEX process is smaller than about 0.25 micron, about 0.22 micron, about 0.2 micron, about 0.18 micron, about 0.16 micron, or about 0.14 micron. In some aspects, the method of the disclosure comprises AEX-Filtration-CEX-MMC.

In some aspects, the present method comprises: (1) Filtration(1)-CEX-Filtration(2)-AEX-Filtration(3)-MMC; (2) Filtration(1)-CEX-Filtration(2)-AEX-MMC-Filtration(3); (3) Filtration(1)-CEX-AEX-Filtration(2)-MMC-Filtration(3); (4) CEX-Filtration(1)-AEX-Filtration(2)-MMC-Filtration(3); (5) Filtration(1)-CEX-Filtration(2)-MMC-Filtration(3)-AEX; (6) Filtration(1)-CEX-Filtration(2)-MMC-AEX-Filtration(3); (7) Filtration(1)-CEX-MMC-Filtration(2)-AEX-Filtration(3); (8) CEX-Filtration(1)-MMC-Filtration(2)-AEX-Filtration(3); (9) Filtration(1)-AEX-Filtration(2)-CEX-Filtration(3)-MMC; (10) Filtration(1)-AEX-Filtration(2)-CEX-MMC-Filtration(3); (11) Filtration(1)-AEX-CEX-Filtration(2)-MMC-Filtration(3); (12) AEX-Filtration(1)-CEX-Filtration(2)-MMC-Filtration(3); (13) Filtration(1)-AEX-Filtration(2)-MMC-Filtration(3)-CEX; (14) Filtration(1)-AEX-Filtration(2)-MMC-CEX-Filtration(3); (15) Filtration(1)-AEX-MMC-Filtration(2)-CEX-Filtration(3); (16) AEX-Filtration(1)-MMC-Filtration(2)-CEX-Filtration(3); (17) Filtration(1)-MMC-Filtration(2)-CEX-Filtration(3)-AEX; (18) Filtration(1)-MMC-Filtration(2)-CEX-AEX-Filtration(3); (19) Filtration(1)-MMC-CEX-Filtration(2)-AEX-Filtration(3); (20) MMC-Filtration(1)-CEX-Filtration(2)-AEX-Filtration(3); (21) Filtration(1)-MMC-Filtration(2)-AEX-Filtration(3)-CEX; (22) Filtration(1)-MMC-Filtration(2)-AEX-CEX-Filtration(3); (23) Filtration(1)-MMC-AEX-Filtration(2)-CEX-Filtration(3); (24) MMC-Filtration(1)-AEX-Filtration(2)-CEX-Filtration(3). In some aspects, the method comprises Filtration (1)-AEX-Filtration (2)-CEX-MMC-Filtration (3). In other aspects, any filtration prior to the CEX process has a bigger filter size compared to a filter size of filtration after the CEX process. In some aspects, the filter size of the filtrations is reduced in or after the CEX process. In some aspects, the filter size prior to the CEX process is bigger than about 0.25 micron, about 0.3 micron, about 0.35 micron, about 0.4 micron, about 0.45 micron, about 0.5 micron, about 0.55 micron, about 0.6 micron, about 0.65 micron, or about 0.7 micron. In other aspects, the filter size of the filtrations in or after the CEX process is smaller than about 0.25 micron, about 0.22 micron, about 0.2 micron, about 0.18 micron, about 0.16 micron, or about 0.14 micron.

In some aspects, the present method comprises: (1) Filtration(1)-CEX-Filtration(2)-AEX-Filtration(3)-MMC-Filtration(4); (2) Filtration(1)-CEX-Filtration(2)-MMC-Filtration(3)-AEX-Filtration(4); (3) Filtration(1)-AEX-Filtration(2)-CEX-Filtration(3)-MMC-Filtration(4); (4) Filtration(1)-AEX-Filtration(2)-MMC-Filtration(3)-CEX-Filtration(4); (5) Filtration(1)-MMC-Filtration(2)-CEX-Filtration(3)-AEX-Filtration(4); or (6) Filtration(1)-MMC-Filtration(2)-AEX-Filtration(3)-CEX-Filtration(4). In other aspects, any filtration prior to the CEX process has a bigger filter size compared to a filter size of filtration in or after the CEX process. In some aspects, the filter size of the various filtrations is reduced in or after the CEX process. In some aspects, the filter size prior to the CEX process is bigger than about 0.25 micron, about 0.3 micron, about 0.35 micron, about 0.4 micron, about 0.45 micron, about 0.5 micron, about 0.55 micron, about 0.6 micron, about 0.65 micron, or about 0.7 micron. In other aspects, the filter size of the filtrations in or after the CEX process is smaller than about 0.25 micron, about 0.22 micron, about 0.2 micron, about 0.18 micron, about 0.16 micron, or about 0.14 micron. In some aspects, the present filtration useful in the process is a sterile filtration. One or more sterile filtrations can be performed within the present methods. In some aspects, at least one, at least two, at least three, at least four, at least five, at least six, at least seven, at least eight, at least nine, at least ten, at least 11, at least 12, at least 13, at least 14, or at least 15 filtrations can be introduced in the present methods. In some aspects, a sterile filtration can be introduced between two chromatographies. In some aspects, filtration can be used right after the harvest. In other aspects, filtration can be used right before formulation.

### III. Samples Comprising EVs

Samples comprising EVs useful for the present methods can be obtained from a various in vitro cell culture or a harvest or a supernatant of the cell culture. In some aspects, the sample comprising EVs can be obtained from a mammalian cell, a bacterial cell, a eukaryotic cell, a prokaryotic cell, a plant cell, an insect cell, or any combination thereof. In some aspects, the sample comprising EVs can be obtained from a mammalian cell. In some aspects, the sample comprising EVs can be obtained from a HEK cell culture. In some aspects, the sample comprising EVs can be a cell culture comprising cells producing EVs.

The present disclosure provides a method for preparing EVs, which can be implemented to purify EVs in a large scale. In some aspects, the method can be applied to purify EVs from a sample with a volume larger than about 1 L, about 5 L, about 10 L, about 15 L about 20 L, about 25 L, about 50 L, about 100 L, about 200 L, about 250 L, about 300 L, about 400 L, about 500 L, about 600 L, about 700 L, about 800 L, about 900 L, about 1000 L, or about 2000 L. In some aspects, the method can be applied to purify EVs from a sample with a volume of about 400 L. In some aspects, the method can be applied to purify EVs from a sample with a volume of about 500 L. In some aspects, the method can be applied to purify EVs from a sample with a volume of about 600 L. In some aspects, the method can be applied to purify EVs from a sample with a volume larger than about 100 L. In some aspects, the method can be applied to purify EVs from a sample with a volume larger than about 200 L. In some aspects, the method can be applied to purify EVs from a sample with a volume larger than about 300 L. In some aspects, the method can be applied to purify EVs from a sample with a volume larger than about 700 L. In some aspects, the method can be applied to purify EVs from a sample with a volume larger than about 1000 L. In some aspects, the method can be applied to purify EVs from a sample with a volume larger than about 1500 L. In some aspects, the method can be applied to purify EVs from a sample with a volume larger than about 2000 L.

In some aspects, the cell culture media useful for the present methods comprises 3D suspension culture comprising high-depth chemically defined media. In some aspects, the method of the present disclosure includes continuous manufacturing processes. In some aspects, the methods comprise continuous manufacturing processes at high cell density (e.g., at least about 50×10⁶ cells/ml, at least about 60×10⁶ cells/ml, at least about 70×10⁶ cells/ml, at least about 80×10⁶ cells/ml, at least about 90×10⁶ cells/ml, at least about 100×10⁶ cells/ml, at least about 110×10⁶ cells/ml, at least about 120×10⁶ cells/ml, at least about 130×10⁶ cells/ml, at least about 140×10⁶ cells/ml, at least about 150×10⁶ cells/ml, at least about 200×10⁶ cells/ml, at least about 250×10⁶ cells/ml, at least about 300×10⁶ cells/ml, at least about 350×10⁶ cells/ml, or at least about 400×10⁶ cells/ml, e.g., 40×10⁶ to 200×10⁶ cell/ml, e.g., 50×10⁶ to 170×10⁶ cell/ml, e.g., 50×10⁶ to 150×10⁶ cell/ml).

In some aspects, each sample has a volume of about 500 L and the 500 L volume sample goes through the purification step (e.g., CEX; AEX; Affinity; CEX and AEX; CEX, AEX, and MMC; or any other combinations) as described herein. In other aspects, the total amount of sample that goes through the purification step for each batch is at least about 5,000 L, at least about 6,000 L, at least about 7,000 L, at least about 8,000 L, at least about 9,000 L, at least about 10,000 L, at least about 11,000 L, at least about 12,000 L, at least about 13,000 L, at least about 14,000 L, or at least about 15,000 L. In other aspects, the total amount of sample that goes through the purification step for each batch is at least about 10,000 L. In other aspects, the total amount of sample that goes through the purification step for each batch is at least about 15,000 L. In other aspects, the total amount of sample that goes through the purification step for each batch is at least about 20,000 L.

In some aspects, the EVs that can be purified by the present methods comprise naturally-occurring EVs, e.g., exosomes. In some aspects, the EVs that can be purified by the present methods comprise engineered EVs, e.g., exosomes. In some aspects, the EVs that can be purified by the present methods comprise surface-engineered EVs, e.g., exosomes. In some aspects, the EVs that can be purified by the present methods comprise engineered EVs, e.g., exosomes that contain one or more (heterologous) moieties in the lumen of the EVs, e.g., exosomes (e.g., encapsulated in the EVs). In some aspects, the EVs that can be purified by the present methods comprise engineered EVs that contain one or more (heterologous) moieties linked to a moiety on the exterior surface of the EVs. In some aspects, the EVs that can be purified by the present methods comprise engineered EVs that contain one or more (heterologous) moieties linked to a moiety on the luminal surface of the EVs.

In other aspects, the EVs from the producer cell can have a longest dimension of from about 20 to about 1000 nm. In some aspects, the EVs from the producer cell can have a longest dimension of from about 20 to about 900 nm, from about 20 to about 800 nm, from about 20 to about 700 nm, from about 20 to about 600 nm, from about 20 to about 500 nm, from about 20 to about 400 nm, from about 20 to about 350 nm, from about 20 to about 300 nm, from about 20 to about 290 nm, from about 20 to about 280 nm, from about 20 to about 270 nm, from about 20 to about 260 nm, from about 20 to about 250 nm, from about 20 to about 240 nm, from about 20 to about 230 nm, from about 20 to about 220 nm, from about 20 to about 210 nm, from about 20 to about 200 nm, from about 20 to about 190 nm, from about 20 to about 180 nm, from about 20 to about 170 nm, about 20 to about 160 nm, from about 20 to about 150 nm, from about 20 to about 140 nm, about 20 to about 130 nm, from about 20 to about 120 nm, In some aspects, the EVs from the producer cell can have a longest dimension of from about 20 to about 110 nm, from about 20 to about 100 nm, from about 20 to about 90 nm, In some aspects, the EVs from the producer cell can have a longest dimension of from about 20 to about 80 nm, from about 20 to about 70 nm, from about 20 to about 60 nm, from about 20 to about 50 nm, from about 20 to about 40 nm, from about 20 to about 30 nm, from about 30 to about 300 nm, from about 30 to about 290 nm, from about 30 to about 280 nm, from about 30 to about 270 nm, from about 30 to about 260 nm, from about 30 to about 250 nm, from about 30 to about 240 nm, from about 30 to about 230 nm, from about 30 to about 220 nm, about 30 to about 210 nm, from about 30 to about 200 nm, from about 30 to about 190 nm, from about 30 to about 180 nm, from about 30 to about 170 nm, from about 30 to about 160 nm, from about 30 to about 150 nm, from about 30 to about 140 nm, from about 30 to about 130 nm, from about 30 to about 120 nm, from about 30 to about 110 nm, from about 30 to about 100 nm, from about 30 to about 90 nm, from about 30 to about 80 nm, from about 30 to about 70 nm, or from about 30 to about 60 nm.

In some aspects, EV membranes comprise lipids and/or fatty acids. In some aspects, EV membranes comprise phospholipids, glycolipids, fatty acids, sphingolipids, phosphoglycerides, sterols, cholesterols, and/or phosphatidylserines. In some of these aspects, EV membranes further comprise one or more polypeptides and/or one or more polysaccharides, such as glycan.

In some aspects, EV membranes comprise one or more molecules derived from the producer cell. In some aspects, EVs can be generated in a cell culture system and isolated from the producer cell. In some aspects, EVs can be generated from a perfusion cell culture. In some aspects, EVs can be generated from a batch cell culture. In some aspects, EVs can be generated from a fed batch cell culture. In some aspects, EVs can be generated from suspension or adherent cells. In some aspects, EVs can be generated from a HEK293 cell, a CHO cell, a BHK cell, a PERC6 cell, a Vero cell, a HeLa cell, a sf9 cell, a PC12 cell, a mesenchymal stem cell, a human donor cell, a stem cell, a dendritic cell, an antigen presenting cell, an induced pluripotent stem cell (IPC), a differentiated cell, bacteria, Streptomyces, Drosophila, Xenopus oocytes, Escherichia coli, Bacillus subtilis, yeast, S. cerevisiae, Picchia pastoris, filamentous fungi, Neurospora crassa, and/or Aspergillus nidulans. In some aspects, the producer cell is a HEK293 cell. The process of EV generation would be generally applicable to bioreactor formats including AMBR, shake flasks, SUBs, Waves, Applikons, stirred tanks, CSTRs, adherent cell culture, hollow fibers, iCELLis, microcarriers, and other methods known to those of skill in the art.

The present disclosure also includes extracellular vesicles (EVs) produced by a cell line. The production of extracellular vesicles and maintenance of cell culture conditions are important to maintain viable cell density of a cell culture process and consistently produce high-quality extracellular vesicles over the full length of a cell culture process. In some aspects, the EVs purified by the present methods are produced in a bioreactor. In some aspects, the EVs purified by the present methods are produced in a single-use bioreactor. In some aspects, the EVs purified by the present methods are produced in a perfusion bioreactor. In some aspects, the EVs purified by the present methods are produced in an alternating tangential flow filtration (ATF) perfusion bioreactor. In some aspects, the EVs purified by the present methods are produced in a tangential flow filtration (TFF) perfusion bioreactor. In some aspects, the EVs purified by the present methods are produced in a bioreactor at a viable cell density (VCD) of about 1×10⁶ cells/mL, about 5×10⁶ cells/mL, about 10×10⁶ cells/mL, about 20×10⁶ cells/mL, about 30×10⁶ cells/mL, about 40×10⁶ cells/mL, about 50×10⁶ cells/mL, or about 60×10⁶ cells/mL. In some aspects, the EVs purified by the present methods are produced in a bioreactor at a viable cell density (VCD) of about 60×10⁶ cells/mL. In some aspects, the EVs purified by the present methods are produced in a bioreactor at a viable cell density (VCD) of about 50×10⁶ cells/mL. In some aspects, the EVs purified by the present methods are produced in a bioreactor at a viable cell density (VCD) of from about 0 to about 60×10⁶ cells/mL, from about 1×10⁶ cells/mL to about 60×10⁶ cells/mL, from about 40×10⁶ cells/mL to about 60×10⁶ cells/mL, or from about 50×10⁶ cells/mL to about 60×10⁶ cells/mL.

In some aspects, the EVs purified by the present methods are produced in a bioreactor for about 5 days, about 10 days, about 15 days, about 20 days, about 25 days, or about 30 days. In some aspects, the EVs purified by the present methods are produced in a bioreactor for about 1-30 days, about 1-45 days, about 1-60 days, about 1-10 days, about 5-10 days, or about 1-25 days. In some aspects, the EVs purified by the present methods are produced in a bioreactor for about 1-30 days.

In some other aspects, EVs are modified by altering components of the membrane of the EV. In some of these aspects, EVs are modified by altering the protein, lipid and/or glycan content of the membrane. In other aspects, EVs are engineered to express a scaffold moiety, e.g., Scaffold X, Scaffold Y, or any other moieties. In some aspects, EVs are engineered to express a higher number of one or more proteins naturally expressed on the surface of producer cells or EVs.

In some aspects, the producer cells naturally contain one or more polypeptides, and EVs derived from the producer cell also contain the one or more polypeptides. In some aspects, the producer cells are modified to contain one or more polypeptides. In some aspects, the modification comprises modulating expression of the one or more polypeptides through use of agents that alter endogenous gene expression. In some aspects, the modification comprises modulating expression of the one or more polypeptides through introduction of expression constructs or mRNAs that encode the one or more polypeptides. In some aspects, EVs produced by these cells include the one or more polypeptides as a payload.

In some aspects, the EV protein is Scaffold X. In some aspects, EVs comprise one or more polypeptides on their surface. In some aspects, the one or more polypeptides can be CD47, CD55, CD49, CD40, CD133, CD59, glypican-1, CD9, CD63, CD81, integrins, selectins, lectins, cadherins and/or other similar polypeptides known to those of skill in the art. In some aspects, the one or more polypeptides can be a scaffold protein, such as PTGFRN, BSG, IGSF3, IGSF2, ITGB1, ITGA4, SLC3A2, ATP transporter or a fragment thereof. In some aspects, the payload (e.g., IL-12) is fused to Scaffold X, e.g. PTGFRN.

In some aspects, the EV protein is Scaffold Y. In some aspects, the EV protein is polypeptide is BASP1. In some aspects, the one or more polypeptides is a fusion protein comprising the scaffold protein fused to a different protein. In some aspects, the surface protein can be expressed from an exogenous polynucleotide introduced to the producer cells. In some aspects, the surface polypeptide can confer different functionalities to the EV, for example, specific targeting capabilities, delivery functions, enzymatic functions, increased or decreased half-life in vivo, and other desired functionalities known to those of skill in the art.

As previously described, producer cells can be genetically modified to comprise one or more exogenous sequences to produce EVs described herein. The genetically-modified producer cell can contain the exogenous sequence by transient or stable transfection and/or transformation. The exogenous sequence can be transformed as a plasmid. The exogenous sequences can be stably integrated into a genomic sequence of the producer cell, at a targeted site or in a random site. In some aspects, a stable cell line is generated for production of lumen-engineered EVs.

The exogenous sequences can be inserted into a genomic sequence of the producer cell, located within, upstream (5′-end) or downstream (3′-end) of an endogenous sequence encoding an EV protein. Various methods known in the art can be used for the introduction of the exogenous sequences into the producer cell. For example, cells modified using various gene editing methods (e.g., methods using a homologous recombination, transposon-mediated system, loxP-Cre system, CRISPR/Cas9 or TALEN) are within the scope of the present disclosure.

The exogenous sequences can comprise a sequence encoding a scaffold moiety disclosed herein or a fragment or variant thereof. Extra copies of the sequence encoding a scaffold moiety can be introduced to produce an engineered EV described herein (e.g., having a higher density of a scaffold moiety on the exterior surface or on the luminal surface of the EV). An exogenous sequence encoding a modification or a fragment of a scaffold moiety can be introduced to produce a lumen-engineered and/or surface-engineered EV containing the modification or the fragment of the scaffold moiety.

In some aspects, a producer cell disclosed herein is further modified to comprise an additional exogenous sequence. For example, an additional exogenous sequence can be introduced to modulate endogenous gene expression, or produce an EV including a certain polypeptide. In some aspects, the producer cell is modified to comprise two exogenous sequences, one encoding a scaffold moiety (e.g., Scaffold X and/or Scaffold Y), or a variant or a fragment thereof, and the other encoding a molecule linked to the scaffold moiety. In certain aspects, the producer cell can be further modified to comprise an additional exogenous sequence conferring additional functionalities to the EVs. In some aspects, the producer cell is modified to comprise two exogenous sequences, one encoding a scaffold moiety disclosed herein, or a variant or a fragment thereof, and the other encoding a protein conferring the additional functionalities to the EVs. In some aspects, the producer cell is further modified to comprise one, two, three, four, five, six, seven, eight, nine, or ten or more additional exogenous sequences.

In some aspects, EVs of the present disclosure (e.g., surface-engineered and/or lumen-engineered EVs) can be produced from a cell transformed with a sequence encoding a full-length, mature scaffold moiety disclosed herein. Any of the scaffold moieties described herein can be expressed from a plasmid, an exogenous sequence inserted into the genome or other exogenous nucleic acid, such as a synthetic messenger RNA (mRNA).

In certain aspects, the one or more moieties are introduced into the EVs by transfection. In some aspects, the one or more moieties can be introduced into the EVs using synthetic macromolecules such as cationic lipids and polymers (Papapetrou et al., Gene Therapy 12: S118-S130 (2005)). In certain aspects, chemicals such as calcium phosphate, cyclodextrin, or polybrene, can be used to introduce the one or more moieties to the EVs.

In other aspects, one or more scaffold moieties are expressed in the membrane of the EVs by recombinantly expressing the scaffold moieties in the producer cells. The EVs obtained from the producer cells can be further modified to be conjugated to a chemical compound, a nucleic acid, a peptide, a protein, or a linker. In other aspects, the scaffold moiety, e.g., Scaffold X and/or Scaffold Y, is deglycosylated. In some aspects, the scaffold moiety, Scaffold X and/or Scaffold Y, is highly glycosylated, e.g., higher than naturally-occurring Scaffold X and/or Scaffold Y under the same condition.

In certain aspects, one or more moieties can be introduced into the EVs directly after exosome production e.g., loaded into the EVs: for example, passive diffusion, electroporation, chemical or polymeric transfection, viral transduction, mechanical membrane disruption or mechanical shear, or any combination thereof. In some aspects, the one or more moieties and the EV, e.g., exosome, of the present disclosure can be incubated in an appropriate buffer during loading or encapsulation. The term “encapsulated”, or grammatically different forms of the term (e.g., encapsulation, or encapsulating), refers to a status or process of having a first moiety (e.g., STING agonist) inside a second moiety (e.g., an EV, e.g., exosome) without chemically or physically linking the two moieties. In some aspects, the term “encapsulated” can be used interchangeably with “in the lumen of” or “loaded”. Non-limiting examples of encapsulating a first moiety (e.g., STING agonist) into a second moiety (e.g., EVs, e.g., exosomes) are disclosed elsewhere herein. In some aspects, the moiety that can be encapsulated or loaded in the EVs includes a STING agonist. STING agonists refer to an agent that activates a STING pathway. Activation of the STING pathway in DCs results in Type I IFN and pro inflammatory cytokine production via TBK1, IRF3, and NF-κB signaling. Binding of IFN to their receptors on cells results in activation of IFN-stimulated response elements and the transcription of IFN-sensitive genes that result in the immune and inflammatory response. IFN signaling also cross-primes DCs to promote antigen persistence, alters the antigen repertoire available for MHCI presentation, enhances MHCI presentation of antigens, and increases the overall surface expression of MHCI, MHCII, and co-stimulatory molecules CD40, CD80, and CD86. These actions result in increased priming of tumor specific CD8+ T cells and initiation of the adaptive immune response.

In some aspects, a STING agonist useful for the EVs of the present disclosure comprises a cyclic dinucleotide (CDN) and/or a non-cyclic nucleotide. STING agonists used in this disclosure can be cyclic purine dinucleotides such as, but not limited to, cGMP, cyclic di-GMP (c-di-GMP), cAMP, cyclic di-AMP (c-di-AMP), cyclic-GMP-AMP (cGAMP), cyclic di-IMP (c-di-IMP), cyclic AMP-IMP (cAIMP), and any analogue thereof, which are known to stimulate or enhance an immune or inflammation response in a patient. The CDNs may have 2′2′, 2′3′, 2′5′, 3′3′, or 3′5′ bonds linking the cyclic dinucleotides, or any combination thereof. Further non-limiting examples of STING agonists that can be used with the present disclosure include: DMXAA, STING agonist-1, ML RR-S2 CDA, ML RR-S2c-di-GMP, ML-RR-S2 cGAMP, 2′3′-c-di-AM(PS)2, 2′3′-cGAMP, 2′3′-cGAMPdFHS, 3′3′-cGAMP, 3′3′-cGAMPdFSH, cAIMP, cAIM(PS)2, 3′3′-cAIMP, 3′3′-cAIMPdFSH, 2′2′-cGAMP, 2′3′-cGAM(PS)2, 3′3′-cGAMP, and combinations thereof. Non-limiting examples of the STING agonists can also be found at U.S. Pat. No. 9,695,212, WO 2014/189805 A1, WO 2014/179335 A1, WO 2018/100558 A1, U.S. Pat. No. 10,011,630 B2, WO 2017/027646 A1, WO 2017/161349 A1, and WO 2016/096174 A1, each of which is incorporated by reference in its entirety.

Cyclic purine dinucleotides can be modified via standard organic chemistry techniques to produce analogues of purine dinucleotides. Suitable purine dinucleotides include, but are not limited to, adenine, guanine, inosine, hypoxanthine, xanthine, isoguanine, or any other appropriate purine dinucleotide known in the art. The cyclic dinucleotides may be modified analogues. Any suitable modification known in the art may be used, including, but not limited to, phosphorothioate, biphosphorothioate, fluorinate, and difluorinate modifications.

Non cyclic dinucleotide agonists may also be used, such as 5,6-Dimethylxanthenone-4-acetic acid (DMXAA), or any other non-cyclic dinucleotide agonist known in the art.

It is contemplated that any STING agonist can be used. Among the STING agonists are DMXAA, STING agonist-1, ML RR-S2 CDA, ML RR-S2c-di-GMP, ML-RR-S2 cGAMP, 2′3′-c-di-AM(PS)2, 2′3′-cGAMP, 2′3′-cGAMPdFHS, 3′3′-cGAMP, 3′3′-cGAMPdFSH, cAIMP, cAIM(PS)2, 3′3′-cAIMP, 3′3′-cAIMPdFSH, 2′2′-cGAMP, 2′3′-cGAM(PS)2, 3′3′-cGAMP, c-di-AMP, 2′3′-c-di-AMP, 2′3′-c-di-AM(PS)2, c-di-GMP, 2′3′-c-di-GMP, c-di-IMP, c-di-UMP or any combination thereof. In some aspects, the STING agonist is 3′3′-cAIMPdFSH, alternatively named 3-3 cAIMPdFSH. Additional STING agonists known in the art can also be used.

In some aspects, one or more moieties can be introduced into the EVs via an anchoring moiety, e.g., a lipid anchor, e.g., loaded into the EVs: In other aspects, the lipid anchor can be any lipid anchor known in the art, e.g., palmitic acid or glycosylphosphatidylinositols. Under unusual circumstances, e.g., by using a culture medium where myristic acid is limiting, some other fatty acids including shorter-chain and unsaturated, can be attached to the N-terminal glycine. For example, in BK channels, myristate has been reported to be attached posttranslationally to internal serine/threonine or tyrosine residues via a hydroxyester linkage. Membrane anchors known in the art are presented in the following table:

| Modification | Modifying Group |
|---|---|
| S-Palmitoylation | 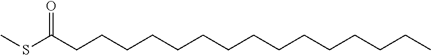 [SMILES: CCCCCCCCCCCCCCCC(=O)SC] |
| M-Palmitoylation | 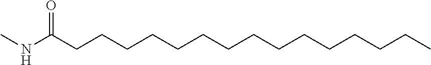 [SMILES: CCCCCCCCCCCCCCCC(=O)NC] |
| N-Myristoylation | 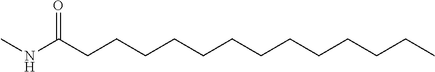 [SMILES: CCCCCCCCCCCCCC(=O)NC] |
| O-Acylation | 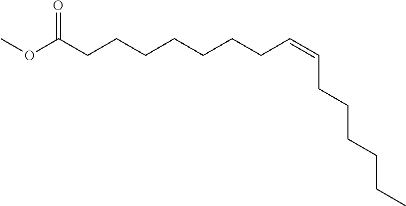 [SMILES: CCCCCC/C=C\CCCCCCCC(=O)OC] |
| Farnesylation | 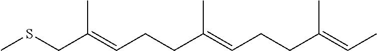 [SMILES: C/C=C(\C)CC/C=C(\C)CC/C=C(\C)CSC] |
| Geranylgeranylation | 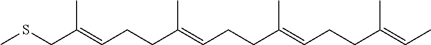 [SMILES: C/C=C(\C)CC/C=C(\C)CC/C=C(\C)CC/C=C(\C)CSC] |
| Cholesterol | 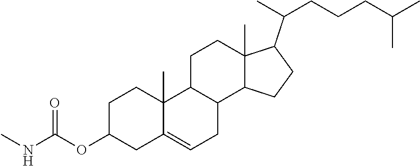 [SMILES: CNC(=O)OC1CCC2(C)C(=CCC3C2CCC2(C)C(C(C)CCCC(C)C)CCC32)C1] |

III.A. Linkers

In some aspects, a payload, e.g., one or more moieties, can be linked to a scaffold moiety or an anchoring moiety either chemically or non-chemically. In some aspects, a biologically active molecule is linked to a scaffold moiety or an anchoring moiety or an EV via a chemical linker, e.g., a maleimide moiety, a sulfhydryl linker, etc.

In some aspects, a payload is linked to a scaffold moiety or an anchoring moiety on the exterior surface of the EV. In some aspects, the payload is linked to the scaffold moiety or an anchoring moiety on the luminal surface of the EV. In some aspects, the scaffold moiety or an anchoring moiety comprises sterol, GM1, a lipid, a vitamin, a small molecule, a peptide, or a combination thereof. In some aspects, the scaffold moiety or an anchoring moiety comprises cholesterol. In some aspects, the scaffold moiety or an anchoring moiety comprises a phospholipid, a lysophospholipid, a fatty acid, a vitamin (e.g., vitamin D and/or vitamin E), or any combination thereof. In some aspects, the payload is linked to the scaffold moiety or an anchoring moiety by a linker.

In some aspects, a linker can comprise a cholesterol moiety. See, e.g., US 2008/0085869 A1, which is herein incorporated by reference in its entirety.

In some aspects, one or more linkers comprise smaller units (e.g., HEG, TEG, glycerol, C2 to C12 alkyl, and the like) linked together. In some aspects, the linkage is an ester linkage (e.g., phosphodiester or phosphorothioate ester) or other linkage. Examples of non-cleavable linkers that can be used with the present disclosure are known in the art, see, e.g., U.S. Pat. No. 7,569,657 B2; U.S. Pat. No. 8,465,730 B1; U.S. Pat. No. 7,087,229 B2; and U.S. Publ. No. 2014/0193849 A1, each of which is herein incorporated by reference in its entirety. In some aspects, the linker can be, e.g., maleimido caproyl (MC), maleimido propanoyl (MP), methoxyl polyethyleneglycol (MPEG), succinimidyl 4-(N-maleimidomethyl)-cyclohexane-1-carboxylate (SMCC), m-maleimidobenzoyl-N-hydroxysuccinimide ester (MBS), succinimidyl 4-(p-maleimidophenyl)butyrate (SMPB), N-succinimidyl(4-iodoacetyl)aminobenzonate (SIAB), succinimidyl 6-[3-(2-pyridyldithio)-propionamide]hexanoate (LC-SPDP), 4-succinimidyloxycarbonyl-alpha-methyl-alpha-(2-pyridyldithio)toluene (SMPT), etc. (see, e.g., U.S. Pat. No. 7,375,078, which is herein incorporated by reference in its entirety).

In some aspects, the linker comprises acrylic phosphoramidite (e.g., ACRYDITE™), adenylation, azide (NHS Ester), digoxigenin (NHS Ester), cholesterol-TEG, I-LINKER™, an amino modifier (e.g., amino modifier C6, amino modifier C12, amino modifier C6 dT, or Uni-Link™ amino modifier), alkyne, 5′ Hexynyl, 5-Octadiynyl dU, biotinylation (e.g., biotin, biotin (Azide), biotin dT, biotin-TEG, dual biotin, PC biotin, or desthiobiotin), thiol modification (thiol modifier C3 S—S, dithiol or thiol modifier C6 S—S), or any combination thereof. In some aspects, the linker is a cleavable linker. In some aspects, the linker comprises valine-alanine-p-aminobenzylcarbamate or valine-citrulline-p-aminobenzylcarbamate. In some aspects, the linker comprises (i) a maleimide moiety and (ii) valine-alanine-p-aminobenzylcarbamate or valine-citrulline-p-aminobenzylcarbamate.

### IV. Extracellular Vesicles Purified by Present Methods

The present disclosure also includes extracellular vesicles (EVs), e.g., exosomes, purified by the present disclosure. In some aspects, the EVs purified by the present methods include lower impurities, e.g., total nucleic acid impurities, than EVs purified by a process that does not comprise contacting a chromatography resin with a nuclease wash buffer.

In some aspects, the present disclosure provides a pharmaceutical composition comprising the purified EVs described herein and a pharmaceutically acceptable carrier. In some aspects, the present disclosure provides a composition comprising EVs and nucleic acid molecule impurities, wherein the nucleic acid molecule impurities are lower than a reference EV composition purified by a process that does not comprise contacting a chromatography resin with a nuclease wash buffer. In some aspects, the present disclosure provides a composition comprising EVs and nucleic acid molecule impurities, wherein the nucleic acid molecule impurities are at least about 5%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, or at least about 40% lower in the purified EV composition compared to a reference EV composition purified by a process that does not comprise contacting a chromatography resin with a nuclease wash buffer.

In some aspects, the nucleic acid molecule impurities are at least about 5%, e.g., 5% to 10%, 5% to 20%, 5% to 25%, or 5% to 30%, lower in the purified EV composition compared to a reference EV composition purified by a process that does not comprise contacting a chromatography resin with a nuclease wash buffer. In some aspects, the nucleic acid molecule impurities are at least about 10%, e.g., 10% to 15%, 10% to 20%, 10% to 25%, 10% to 30%, 10% to 35%, 10% to 30%, 10% to 95%, 20% to 90%, 50% to 90%, or 80% to 90% lower in the purified EV composition compared to a reference EV composition purified by a process that does not comprise contacting a chromatography resin with a nuclease wash buffer. In some aspects, the protein impurities are at least about 11% lower in the purified EV composition compared to a reference EV composition purified by a process that does not comprise contacting a chromatography resin with a nuclease wash buffer. In some aspects, the nucleic acid molecule impurities are at least about 12% lower in the purified EV composition compared to a reference EV composition purified by a process that does not comprise contacting a chromatography resin with a nuclease wash buffer. In some aspects, the nucleic acid molecule impurities are at least about 13% lower in the purified EV composition compared to a reference EV composition purified by a process that does not comprise contacting a chromatography resin with a nuclease wash buffer. In some aspects, the nucleic acid molecule impurities are at least about 14% lower in the purified EV composition compared to a reference EV composition purified by a process that does not comprise contacting a chromatography resin with a nuclease wash buffer. In some aspects, the nucleic acid molecule impurities are at least about 15%, e.g., 15% to 20%, 15% to 25%, 15% to 30%, 15% to 35%, 15% to 40%, 20% to 25%, 20% to 30%, 20% to 35%, or 20% to 40%, lower in the purified EV composition compared to a reference EV composition purified by a process that does not comprise contacting a chromatography resin with a nuclease wash buffer.

In some aspects, compositions comprising the purified EVs has an EV concentration that is approximately the same as the concentration of EVs in a reference composition comprising EVs purified by a process that does not comprise contacting a chromatography resin with a nuclease wash buffer. In some aspects, compositions comprising the purified EVs has an EV concentration that is more than about 99% of the concentration of EVs in a reference composition comprising EVs purified by a process that does not comprise contacting a chromatography resin with a nuclease wash buffer. In some aspects, compositions comprising the purified EVs has an EV concentration that is more than about 98% of the concentration of EVs in a reference composition comprising EVs purified by a process that does not comprise contacting a chromatography resin with a nuclease wash buffer. In some aspects, compositions comprising the purified EVs has an EV concentration that is more than about 97% of the concentration of EVs in a reference composition comprising EVs purified by a process that does not comprise contacting a chromatography resin with a nuclease wash buffer. In some aspects, compositions comprising the purified EVs has an EV concentration that is more than about 96% of the concentration of EVs in a reference composition comprising EVs purified by a process that does not comprise contacting a chromatography resin with a nuclease wash buffer. In some aspects, compositions comprising the purified EVs has an EV concentration that is more than about 95% of the concentration of EVs in a reference composition comprising EVs purified by a process that does not comprise contacting a chromatography resin with a nuclease wash buffer. In some aspects, compositions comprising the purified EVs has an EV concentration that is more than about 90% of the concentration of EVs in a reference composition comprising EVs purified by a process that does not comprise contacting a chromatography resin with a nuclease wash buffer. In some aspects, compositions comprising the purified EVs has an EV concentration that is more than about 85% of the concentration of EVs in a reference composition comprising EVs purified by a process that does not comprise contacting a chromatography resin with a nuclease wash buffer. In some aspects, compositions comprising the purified EVs has an EV concentration that is more than about 80% of the concentration of EVs in a reference composition comprising EVs purified by a process that does not comprise contacting a chromatography resin with a nuclease wash buffer.

In some aspects, compositions comprising the purified EVs have a higher potency than a reference composition comprising EVs purified by a process that does not comprise contacting a chromatography resin with a nuclease wash buffer. In some aspects, the potency of the composition comprising the purified EVs is at least about 5%, e.g., 5% to 10%, 5% to 15%, 5% to 20%, 5% to 25%, 5% to 30%, 5% to 35%, 5% to 40%, 5% to 45%, 5% to 50%, e.g., 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% higher than that of a reference composition comprising EVs purified by a process that does not comprise contacting a chromatography resin with a nuclease wash buffer. In some aspects, the potency of the composition comprising the purified EVs is at least about 10%, e.g., 10% to 15%, 10% to 20%, 10% to 25%, 10% to 30%, 10% to 35%, 10% to 40%, 10% to 45%, 10% to 50%, 10% to 55%, or 10% to 60%, e.g., 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, or 60%, higher than that of a reference composition comprising EVs purified by a process that does not comprise contacting a chromatography resin with a nuclease wash buffer. In some aspects, the potency of the composition comprising the purified EVs is at least about 11% higher than that of a reference composition comprising EVs purified by a process that does not comprise contacting a chromatography resin with a nuclease wash buffer. In some aspects, the potency of the composition comprising the purified EVs is at least about 15%, e.g., 15% to 20%, 15% to 25%, 15% to 30%, 15% to 35%, 15% to 40%, 15% to 45%, 15% to 50%, 15% to 55%, or 15% to 60%, e.g., 16%, 17%, 18%, 19%, 20%, 21%, 22%, 23%, 24%, 25%, 26%, 27%, 28%, 29%, 30%, 35%, 40%, 45%, 50%, 55%, or 60%, higher than that of a reference composition comprising EVs purified by a process that does not comprise contacting a chromatography resin with a nuclease wash buffer. In some aspects, the potency of the composition comprising the purified EVs is at least about 20%, e.g., 20% to 25%, 20% to 30%, 20% to 35%, 20% to 40%, 20% to 45%, 20% to 50%, 20% to 55%, or 20% to 60%, e.g., 20%, 21%, 22%, 23%, 24%, 25%, 26%, 27%, 28%, 29%, 30%, 35%, 40%, 45%, 50%, 55%, or 60%, higher than that of a reference composition comprising EVs purified by a process that does not comprise contacting a chromatography resin with a nuclease wash buffer. In some aspects, the potency of the composition comprising the purified EVs is at least about 25%, e.g., 25% to 30%, 25% to 35%, 25% to 40%, 25% to 45%, 25% to 50%, 25% to 55%, or 25% to 60%, e.g., 25%, 26%, 27%, 28%, 29%, 30%, 35%, 40%, 45%, 50%, 55%, 60%, 65%, 70%, 75%, or 80%, higher than that of a reference composition comprising EVs purified by a process that does not comprise contacting a chromatography resin with a nuclease wash buffer. In some aspects, the potency of the composition comprising the purified EVs is at least about 30%, e.g., 30% to 35%, 30% to 40%, 30% to 45%, 30% to 50%, 30% to 55%, or 30% to 60%, e.g., 30%, 35%, 40%, 45%, 50%, 55%, 60%, 65%, 70%, 75%, 80%, 80%, 85%, or 90% higher than that of a reference composition comprising EVs purified by a process that does not comprise contacting a chromatography resin with a nuclease wash buffer. In some aspects, the potency of the composition comprising the purified EVs is at least about 35%, e.g., 35% to 40%, 35% to 45%, 35% to 50%, 35% to 55%, or 35% to 60%, e.g., 35%, 40%, 45%, 50%, 55%, 60%, 65%, 70%, 75%, 80%, 80%, 85%, or 90% higher than that of a reference composition comprising EVs purified by a process that does not comprise contacting a chromatography resin with a nuclease wash buffer. In some aspects, the potency of the composition comprising the purified EVs is at least about 40%, e.g., 40% to 45%, 40% to 50%, 40% to 55%, or 40% to 60%, e.g., 40%, 45%, 50%, 55%, 60%, 65%, 70%, 75%, 80%, 80%, 85%, or 90%, higher than that of a reference composition comprising EVs purified by a process that does not comprise contacting a chromatography resin with a nuclease wash buffer. In some aspects, the potency of the composition comprising the purified EVs is at least about 45%, e.g., 45% to 50%, 45% to 55%, or 45% to 60%, e.g., 45%, 50%, 55%, 60%, 65%, 70%, 75%, 80%, 80%, 85%, or 90% higher than that of a reference composition comprising EVs purified by a process that does not comprise contacting a chromatography resin with a nuclease wash buffer. In some aspects, the potency of the composition comprising the purified EVs is at least about 50% higher than that of a reference composition comprising EVs purified by a process that does not comprise contacting a chromatography resin with a nuclease wash buffer.

In some aspects, the purified EVs according to the present disclosure is at least 75% pure. In some aspects, the purified EVs according to the present disclosure is at least about 80% pure. In some aspects, the purified EVs according to the present disclosure is at least about 85% pure. In some aspects, the purified EVs according to the present disclosure is at least about 90% pure. In some aspects, the purified EVs according to the present disclosure is at least about 95% pure. In some aspects, the purified EVs according to the present disclosure is at least about 96% pure. In some aspects, the purified EVs according to the present disclosure is at least about 97% pure. In some aspects, the purified EVs according to the present disclosure is at least about 98% pure. In some aspects, the purified EVs according to the present disclosure is at least about 99% pure. In some aspects, the purified EVs according to the present disclosure is about 100% pure.

In some aspects, a composition comprising the purified EVs of the present disclosure further comprises a saccharide. In some aspects, a composition comprising the purified EVs of the present disclosure further comprises sodium chloride. In some aspects, a composition comprising the purified EVs of the present disclosure further comprises a potassium phosphate. In some aspects, a composition comprising the purified EVs of the present disclosure further comprises a sodium phosphate. In some aspects, a composition comprising the purified EVs of the present disclosure further comprises one or more of a saccharide, sodium chloride, a potassium phosphate, and a sodium phosphate. In some aspects, a composition comprising the purified EVs of the present disclosure further comprises a saccharide, sodium chloride, a potassium phosphate, and a sodium phosphate.

In some aspects, the present disclosure provides a method of administering a composition comprising purified EVs to a subject in need thereof. In some aspects, the present disclosure provides a method of treating a disease or condition in a subject in need thereof comprising administering to the subject a composition comprising purified EVs.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

The claims in the instant application are different than those of the parent application or other related applications. The Applicant therefore rescinds any disclaimer of claim scope made in the parent application or any predecessor application in relation to the instant application. The Examiner is therefore advised that any such previous disclaimer and the cited references that it was made to avoid, may need to be revisited. Further, the Examiner is also reminded that any disclaimer made in the instant application should not be read into or against the parent application.

### EXAMPLES

The following examples are put forth so as to provide those of ordinary skill in the art with a complete disclosure and description of how to make and use the aspects described herein, and are not intended to limit the scope of the appended claims, nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations can be used, e.g., s or sec, second(s); min, minute(s); h or hr, hour(s).

The aspects described herein employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); AL. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 21th Edition (Easton, Pennsylvania: Mack Publishing Company, 2005); Carey and Sundberg Advanced Organic Chemistry 3rd Ed. (Plenum Press) Vols A and B (1992).

### Example 1: Wash Buffer Assessment of DNA Clearance Processes

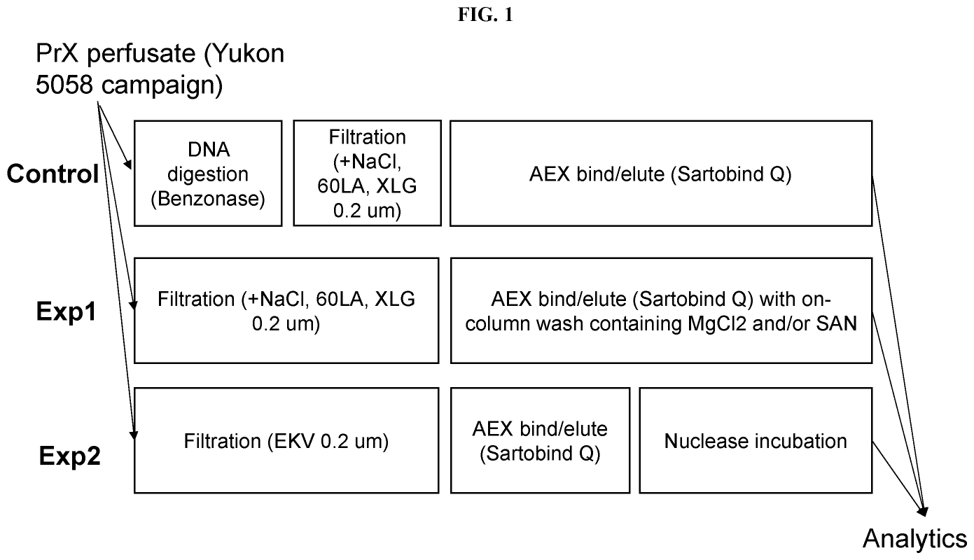

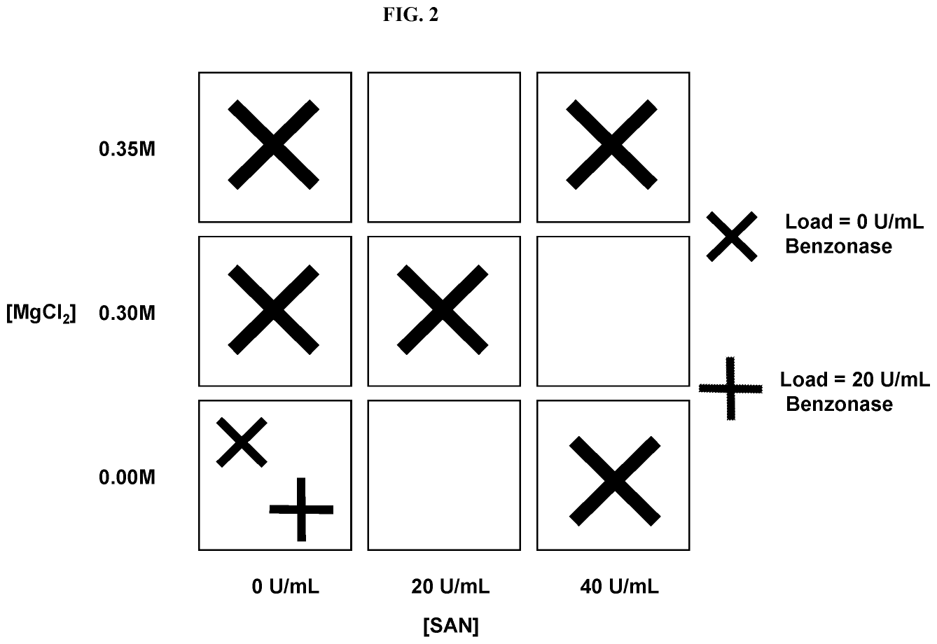

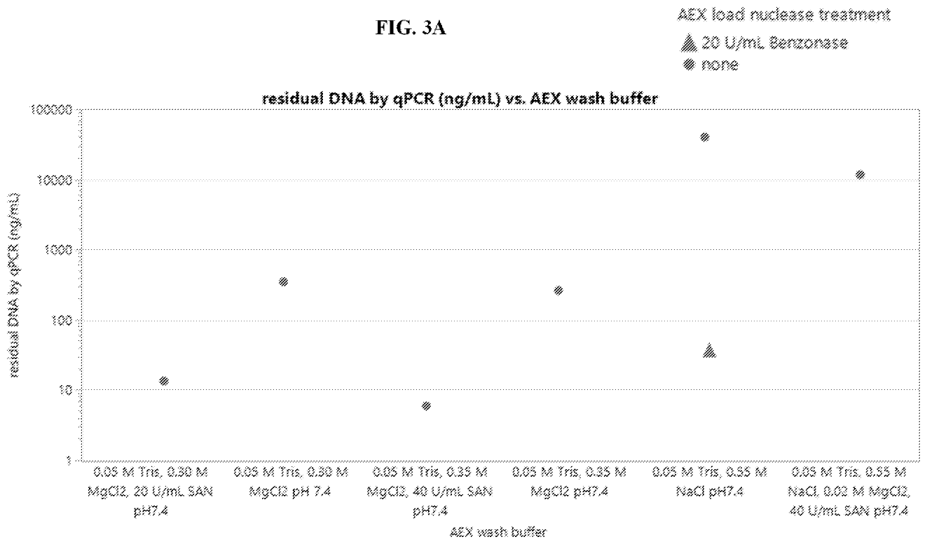

In the present example, the performances of an experimental DNA clearance process and of a control process were evaluated using exosome samples. Schematic representations of the experimental and control DNA clearance processes are presented in FIG. 1 and FIG. 2.

For the control process, an exosome sample was prepared, and DNA digestion was performed using Benzonase (20 U/mL). Following DNA digestion, the sample was filtered (NaCl, 60LA, XLG 0.2 um), and subsequently anion exchange chromatography (AEX) was performed (FIG. 1). More specifically, the filtered exosome sample was loaded onto a SARTOBIND® Q AEX membrane at a concentration of 0.55 M NaCl, and following sample binding, exosome elution was performed using 1.2 M NaCl.

For the experimental processes, exosome samples were prepared, and the sample was filtered (NaCl, 60LA, XLG 0.2 um) (FIG. 1). Subsequently, individual AEX runs were performed on each individual exosome sample, where each of the different individual AEX runs for each individual exosome sample was performed using a wash buffer (FIG. 2). The wash buffers comprised varying amounts of MgCl₂ and salt active nuclease (SAN, ArcticZymes Technologies ASA Norway) (FIG. 2). A pretreatment with benzonase was used as a control (FIG. 2; + sign). For each individual AEX run for each individual exosome sample, the exosome sample was loaded onto a SARTOBIND® Q AEX membrane at a concentration of 0.55 M NaCl, and following sample binding, an on-column wash was performed using a wash buffer (FIG. 2). After the wash step, the exosomes were then eluted from the AEX membrane using 1.2 M NaCl and collected for further processing.

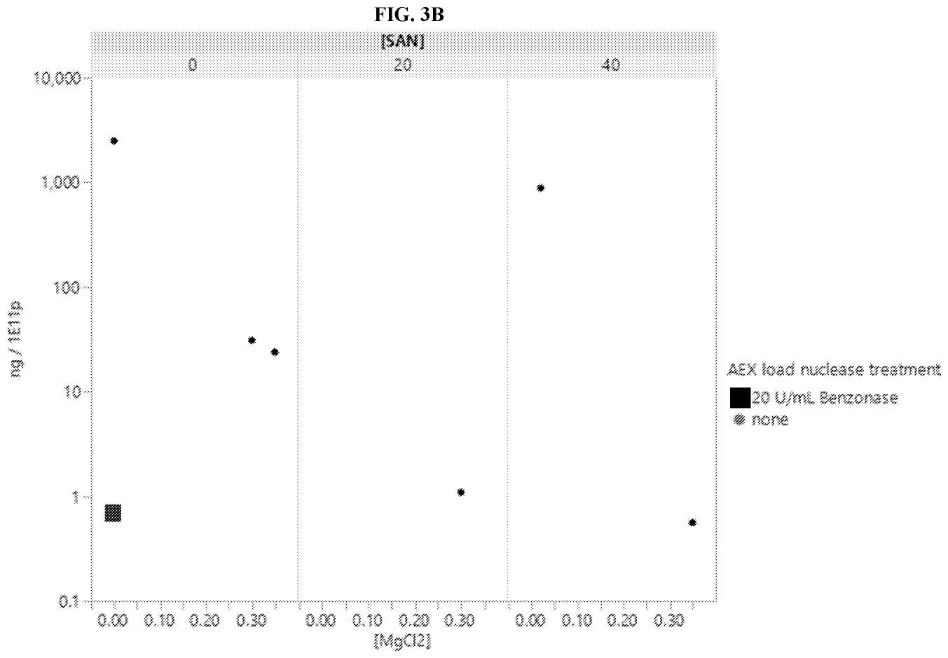

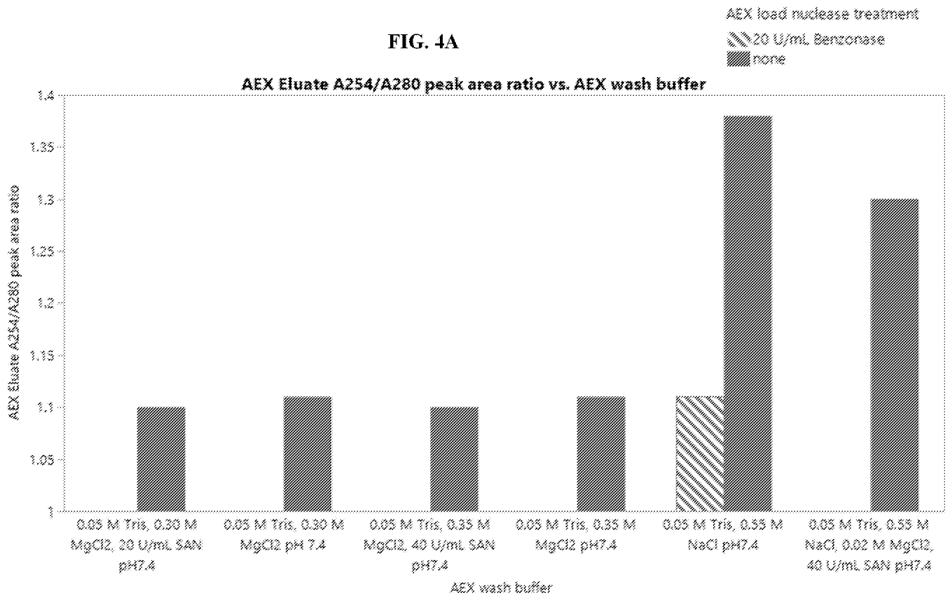

The residual DNA concentrations (ng/mL) of various different AEX eluate samples as compared to the AEX wash buffer used during AEX for a given eluate sample were evaluated (FIGS. 3A-3B). In general, the lowest residual DNA concentrations in AEX sample eluates were obtained when using a wash buffer comprising a combination of MgCl₂ and salt active nuclease (SAN) during the AEX wash step (FIGS. 3A-3B).

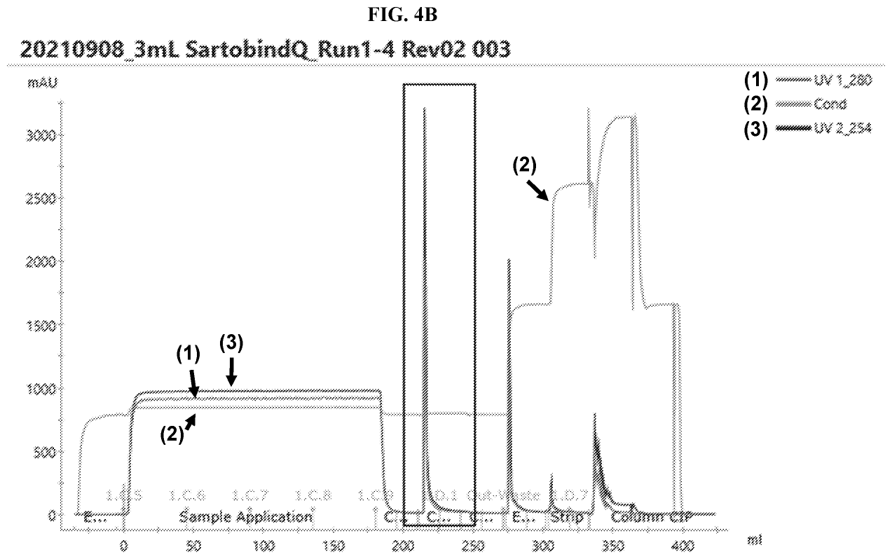

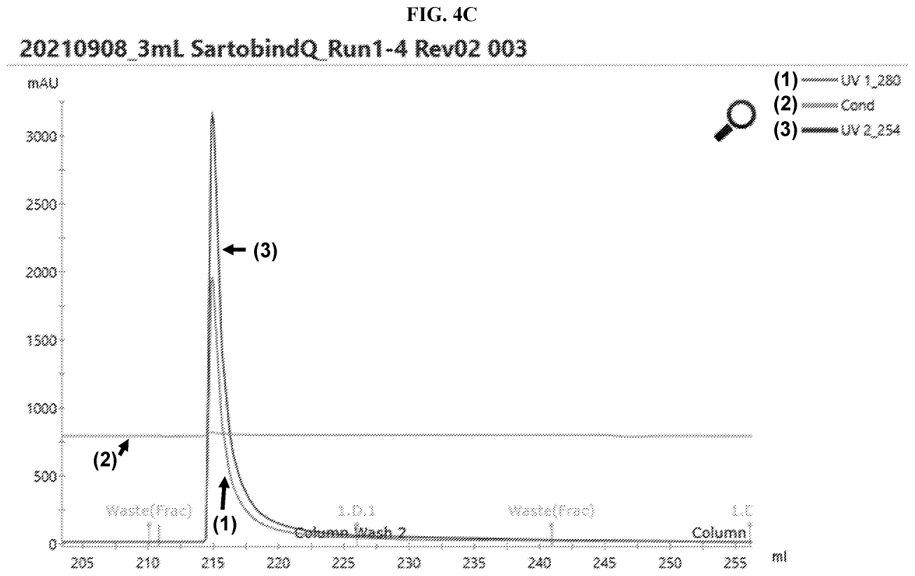

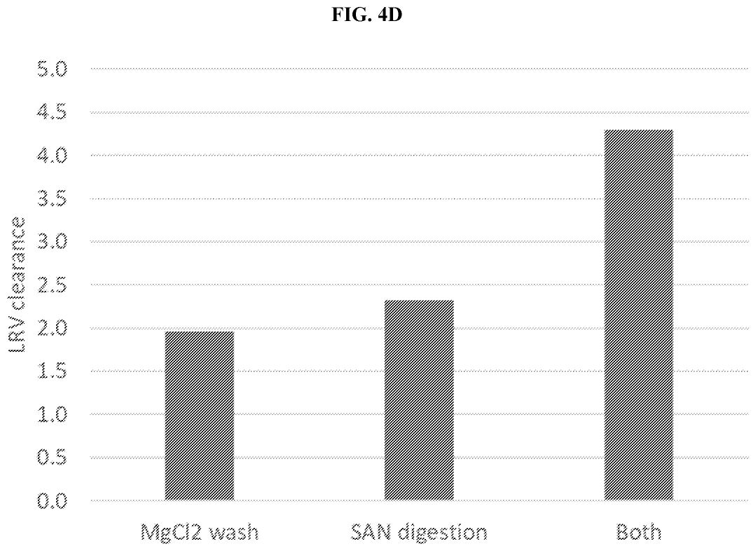

The A254/A280 peak area ratios of various different individual AEX eluate samples as compared to the AEX wash buffer used during AEX for a given eluate sample were evaluated (FIG. 4). In general, a reduction in the A254/A280 peak for AEX eluate samples was observed that was consistent with the reduction in residual DNA as measured by qPCR, i.e., reduced peak areas were observed in AEX sample eluates that were obtained when using a wash buffer comprising a combination of MgCl₂ and salt active nuclease (SAN) during the AEX wash step (FIG. 4).

Clarified harvest material containing exosomes was purified through an anion exchange membrane chromatography process. FIG. 4B shows the full chromatogram. Column flowthrough signals are visualized using UV254 nm, UV280 nm, and conductivity. Chromatography is operated in bind and elute mode, and impurities and product are selectively desorbed from the membrane using isocratic elution with either magnesium chloride or sodium chloride. The MgCl₂ DNA removal wash is shown in the black box, and further in FIG. 4C. The peak desorbed with MgCl₂ in FIG. 3C has an A254/A280 nm ratio of 1.61, which further supports that it is enriched in nucleic acid content.

Clarified harvest material containing exosomes was purified through an anion exchange membrane chromatography process that comprised a 0.35M MgCl₂ excipient wash prior to product elution in a buffer comprised of 1.2M NaCl, pH 7.4. The AEX Eluate was filtered through a 0.2 μm filter and treated with 10 U/mL SAN with an additional 20 μM supplement of MgCl₂. Residual DNA content was measured in samples of the AEX eluate and SAN-treated AEX eluate using qPCR. The residual DNA found in these intermediates were compared to the AEX membrane load material to determine total log (base10) removal of DNA through the unit operation. Both the MgCl₂ membrane wash (desorb bound DNA) and the SAN (digest eluted DNA) contribute to the >4 LRV DNA reduction observed in this process (FIG. 4D).

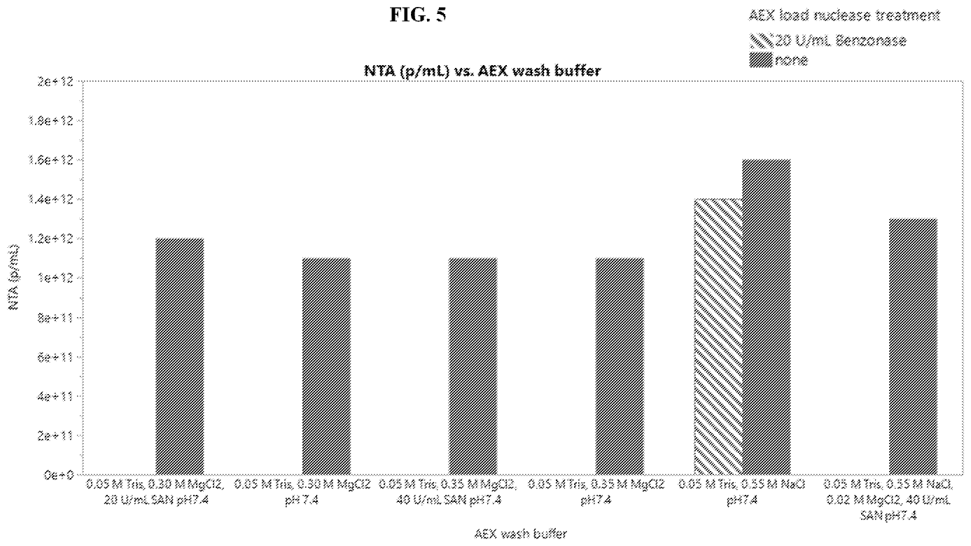

The particle counts of various different AEX eluate samples as compared to the AEX wash buffer used during AEX sample elution were evaluated (FIG. 5). In general, NTA particle counts in AEX eluate samples that were obtained when using a wash buffer comprising MgCl₂ and/or SAN were similar, and further these NTA particle counts were observed to be less than the NTA particle count of the control sample (FIG. 5).

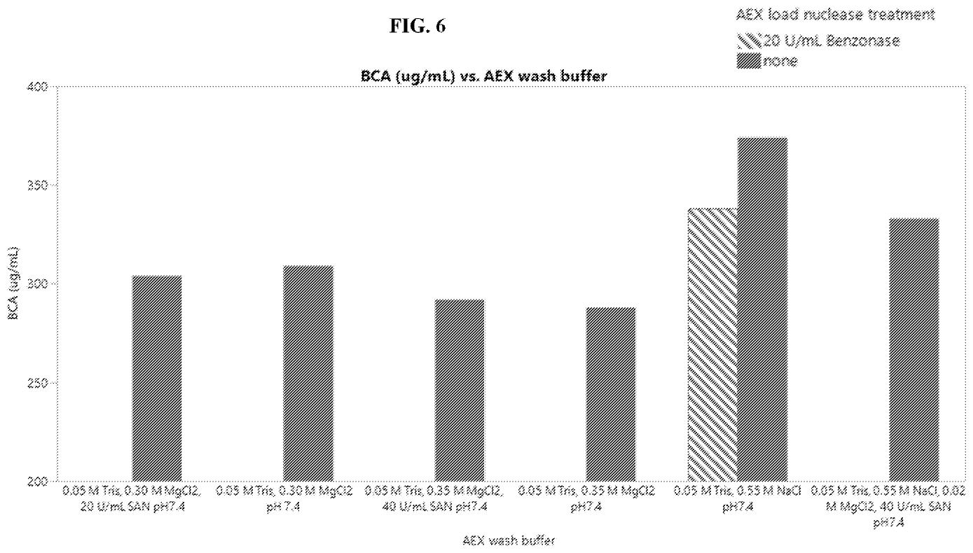

The protein content of various different individual AEX eluate samples as compared to the AEX wash buffer used during AEX was evaluated by BCA assay (FIG. 6). The X-axis of FIG. 6 represents the wash buffer conditions used, and the Y-axis of FIG. 6 represents the protein content (μg/mL) of the various different individual AEX eluate samples as measured by BCA assay. In general, the protein content in AEX eluate samples that were obtained when using a wash buffer comprising MgCl₂ and/or SAN were similar, and further protein content of these samples was observed to be less than the protein content of the control sample (FIG. 6).

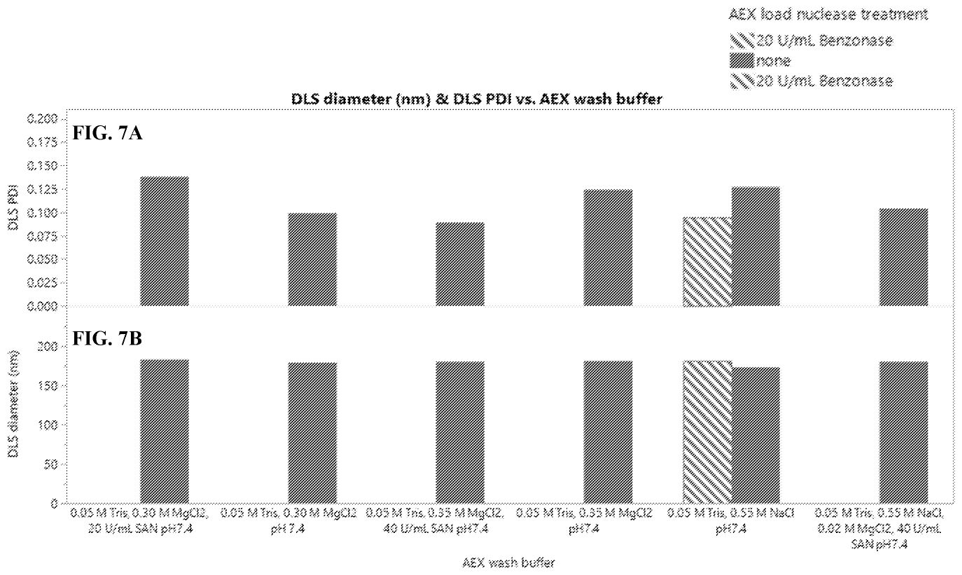

The polydispersity index (PDI) (top panels) and particle diameters (bottom panels) of various different individual AEX eluate samples as compared to the AEX wash buffer used during AEX were evaluated (FIG. 7). Both the PDI and the diameter measurements were performed using dynamic light scattering (DLS). In general, it was observed that there were no substantial changes in either DLS diameter measurements or DLS PDI in AEX eluate samples that were obtained using the various different AEX wash buffer conditions (see FIG. 7).

### Example 2: Nuclease Incubation Assessment of DNA Clearance Processes

The performance of an experimental DNA clearance process and a control process were evaluated using exosome samples. A general schematic representation of the experimental and control DNA clearance processes is presented in FIG. 1.

For the control process, an exosome sample was prepared, and DNA digestion was performed using Benzonase. Following DNA digestion, the sample was filtered (+NaCl, 60LA, XLG 0.2 um) and subsequently anion exchange chromatography (AEX) was performed. More specifically, the exosome sample was loaded onto a SARTOBIND® Q AEX membrane at a concentration of 0.55 M NaCl, and following sample binding, sample elution was performed using 1.2 M NaCl.

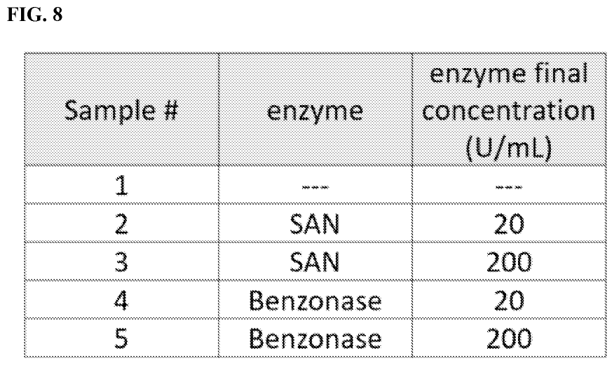

For the experiment process, individual samples of AEX eluates comprising exosomes were prepared by pooling AEX eluates that resulted from untreated load conditions by performing AEX as generally described in Example 1, and diluting the pooled eluates with 50 mM Tris and 1 M MgCl₂ to achieve final concentrations of 550 mM NaCl and 5 mM MgCl₂ in each individual sample. Next, the individual samples were filter adjusted through EKV, which is a sterilizing filter used to reduce the bioburden prior to the nuclease step and reduce risk of bacterial growth during incubation. After filter adjustment, enzyme (SAN or Benzonase) was added to the AEX samples to a final concentration of either 0, 20, or 200 U/mL (FIG. 8). The samples were then incubated for 19 hours at room temperature, and following the incubation period, 10 mM EDTA pH 8.0 was spiked into each sample to quench the nuclease reaction. Samples were frozen and stored prior to sample analysis. Five different individual samples were prepared and evaluated (FIG. 8).

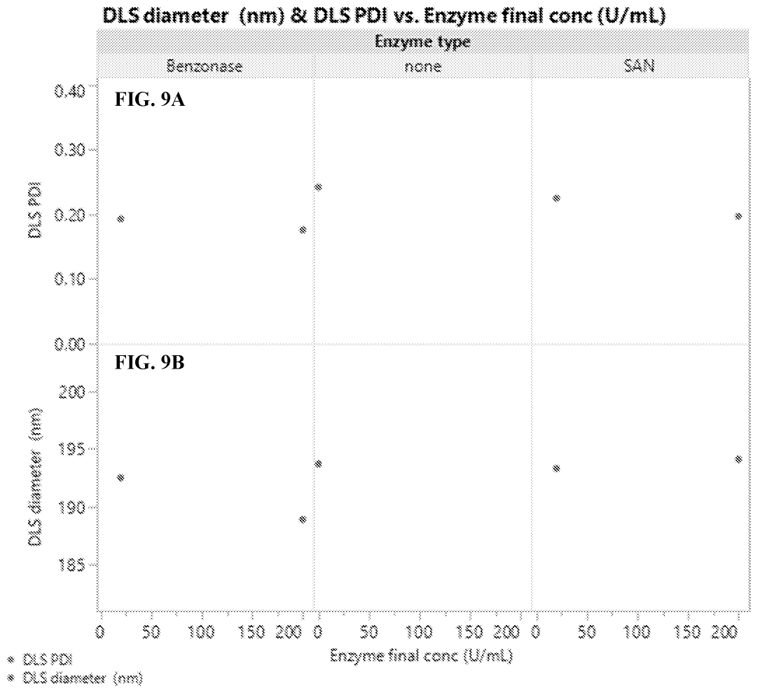

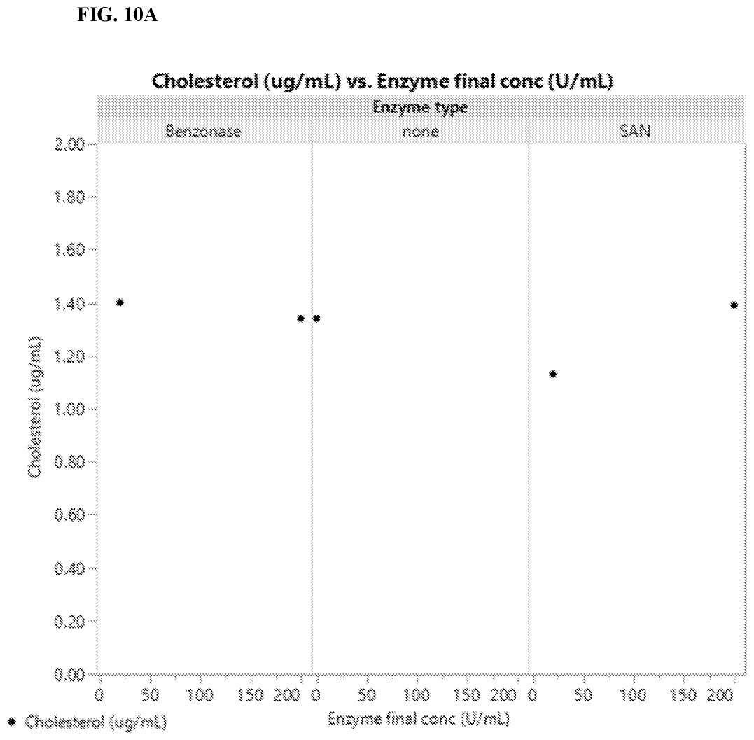

The polydispersity index (PDI) (FIG. 9A) and particle diameters (FIG. 9B) of various different AEX eluate samples as compared to the final enzyme (nuclease) concentration of each of the AEX eluate samples was evaluated. In general, it was observed that there were no substantial changes in either DLS diameter measurements or DLS PDI in AEX eluate samples that were obtained using the various different nuclease treatment conditions.

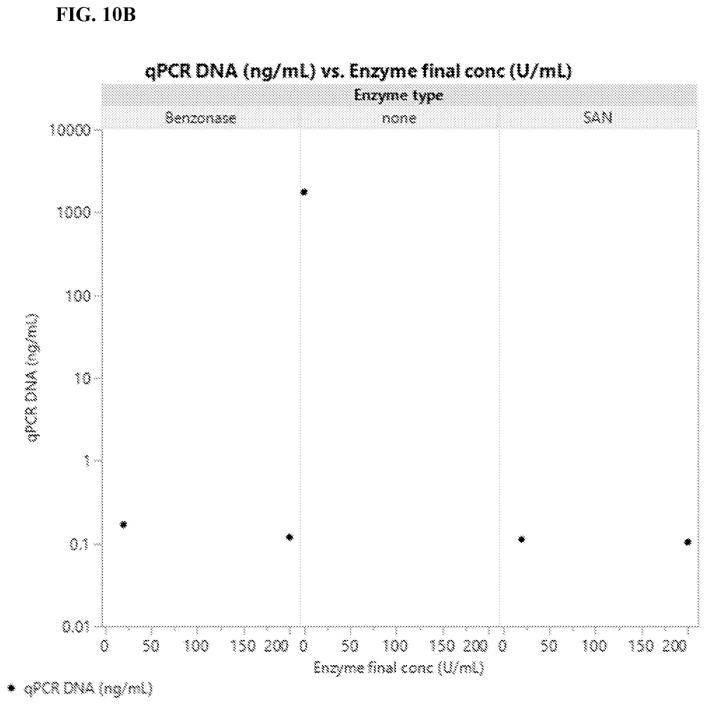

The cholesterol content of the various different individual AEX eluate samples as compared to the final enzyme (nuclease) concentration of each of the AEX eluate samples was evaluated (FIG. 10A). In general, it was observed that neither nuclease treatment resulted in a substantial decrease in cholesterol levels relative to untreated controls.

Residual DNA concentration of various different individual AEX eluate samples as compared to the final enzyme (nuclease) concentration of each of the AEX eluate samples was evaluated by qPCR (FIG. 10B). Treatment using either benzonase or SAN substantially reduced the residual DNA concentration as compared to an untreated sample (FIG. 10B).

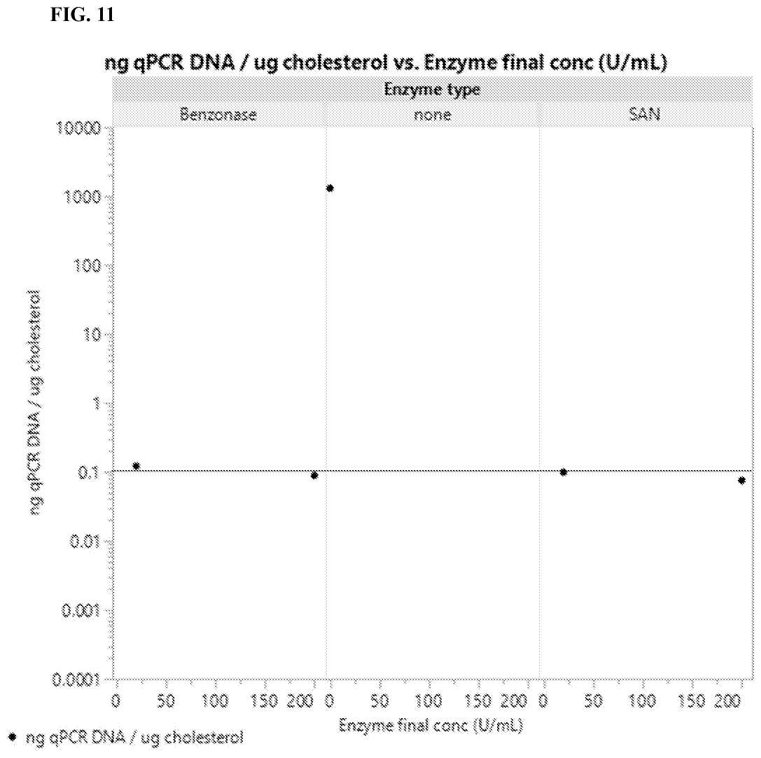

The ratio of residual DNA amount (ng), as measured by qPCR, to the amount of cholesterol (μg) of various different AEX eluate samples as compared to the final enzyme (nuclease) concentration of each of the AEX eluate samples was evaluated using the data as presented in FIGS. 10A-10B (FIG. 11). A 4-log reduction in the DNA amount (ng) per μg cholesterol upon nuclease treatment as compared to the nuclease untreated control condition was observed (FIG. 11).

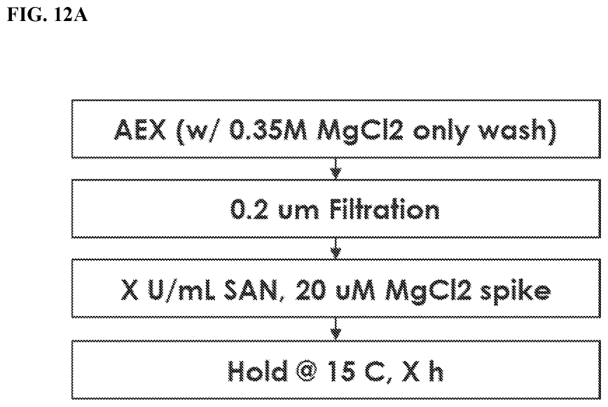

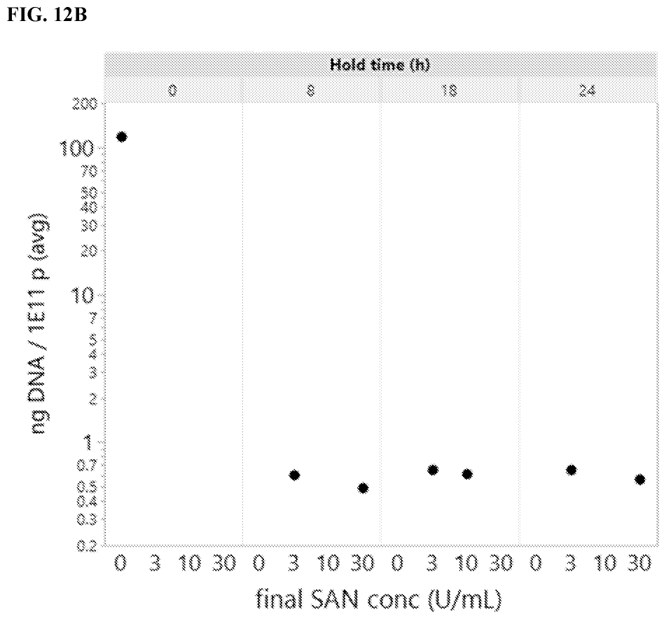

Clarified harvest material containing exosomes was purified through an anion exchange membrane chromatography process that comprised a 0.35M MgCl₂ excipient wash prior to product elution in a buffer comprised of 1.2M NaCl, pH 7.4. AEX eluates were filtered through a 0.2 μm filter and treated with varying amounts of SAN with an additional 20 μM supplement of MgCl₂. All eluates were held at 15° C. for 0, 8, 18, or 14 hours (FIG. 12A). SAN treatment was effective at reducing DNA concentration at each of 3 U/mL, 10 U/mL, and 30 U/mL, and at all timepoints tested. FIG. 12B. Increasing the concentration of SAN beyond 3 U/mL resulted in a modest decrease in the DNA concentration; however, holding the samples for longer than 8 hours had no apparent effect on residual DNA concentration. FIG. 12B.

### Example 3: Large-Scale Nuclease Incubation Assessment of DNA Clearance Processes

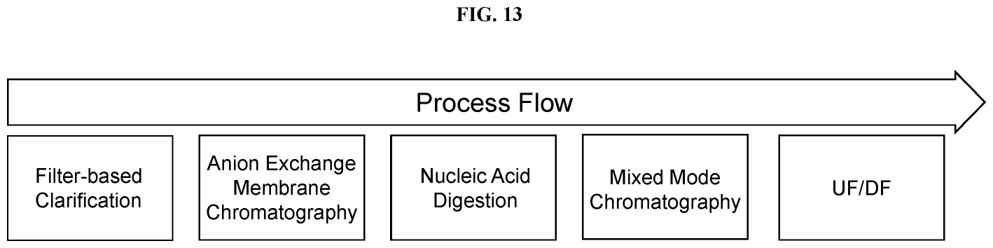

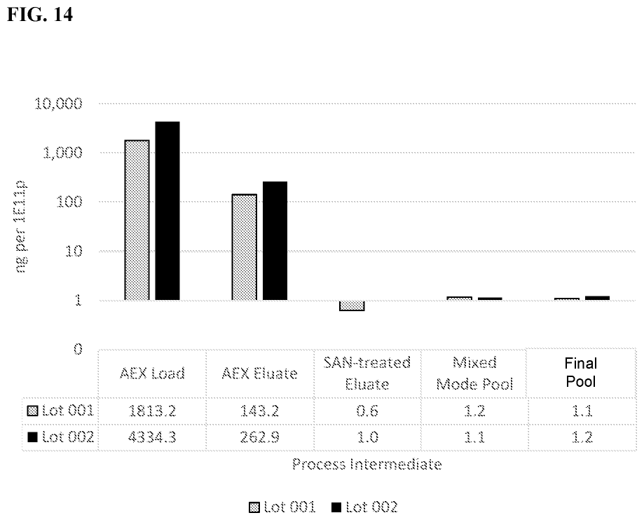

A large-scale method for reducing residual nucleotides was developed. FIG. 13 outlines a process comprising a filter-based clarification step prior to the anion exchange chromatography (AEX) step, followed by nucleic acid digestion, an additional chromatography step (e.g., mixed mode chromatography), and a filtration step. FIG. 14 illustrates that residual DNA levels remain high following AEX chromatography, while subsequent SAN-treatment significantly reduces residual DNA by greater than three logs, which was consistent across two separate lots. Further purification using mixed mode chromatography and filtration did not further reduce residual DNA levels.

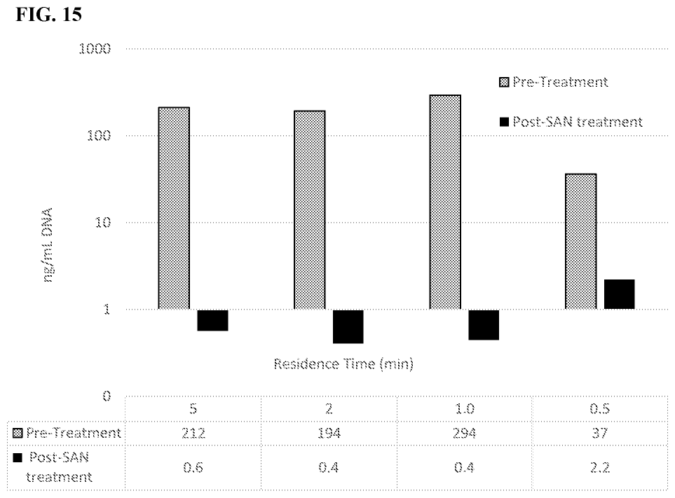

Clarified harvest material containing exosomes was purified through an anion exchange membrane chromatography process that comprised a 0.35M MgCl₂ excipient wash prior to product elution in a buffer comprised of 1.2M NaCl, pH 7.4. Four iterations of the purification were performed; each with a different 0.35M MgCl₂ wash residence time. The residence times ranged from 5, 2, 1 and 0.5 minutes (FIG. 15). AEX eluates were filtered through a 0.2 μm filter and treated with SAN in the presence of 20 μM MgCl₂. All eluates were held at 15° C. for 18 hours at 15-25° C. These data illustrate robust clearance to a range of residence times, showing that with short residence times, the residual DNA in the post-SAN treatment can be elevated as compared to longer residence times (FIG. 15).

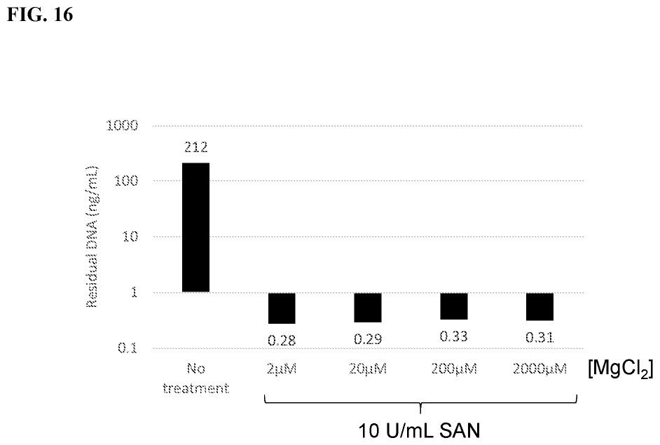

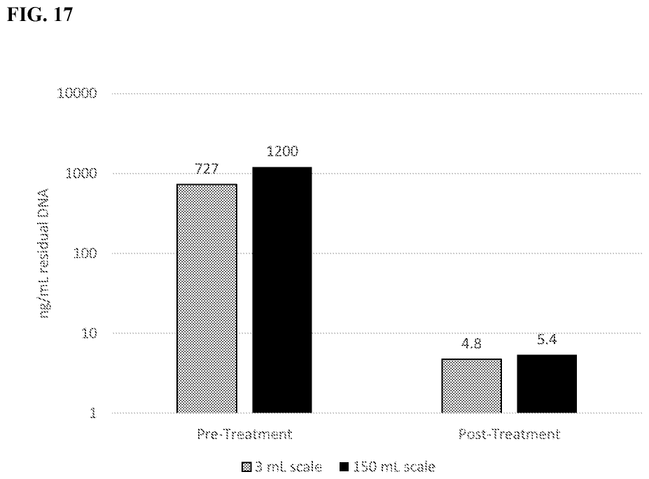

Next, clarified harvest material containing exosomes was purified through an anion exchange membrane chromatography process that comprised a 0.35M MgCl₂ excipient wash prior to product elution in a buffer comprised of 1.2M NaCl, pH 7.4. Prior to treatment with SAN, the AEX eluate was filtered through a 0.2 μm filter and spiked with either 2, 20, 200, or 2000 μM MgCl₂. 10 U/mL of SAN was added to each MgCl₂ condition, and held for 16 hours at 15-25° C. SAN was observed to be active across a three-log range of Mg⁺⁺ cofactor concentration (FIG. 16).

To test the potential effect of AEX membrane volume on SAN treatment, clarified harvest material containing exosomes was purified using either a 3 mL or 150 mL anion exchange membrane device. The devices were scaled by maintaining the volumetric load challenge per mL of resin, and each phase of the separation was scaled per contact volume and residence time. AEX eluates were filtered through a 0.2 μm filter and treated with 10 U/mL SAN with an additional 20 μM supplement of MgCl₂. All eluates were held at 15° C. for 18 hours at 15-25° C. Residual DNA reduction following SAN treatment was observed using either the 3 mL or 150 mL anion exchange membrane device (FIG. 16).

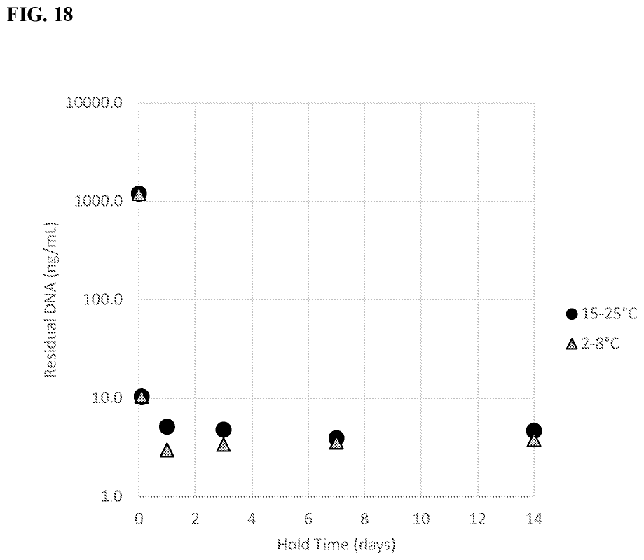

Exosomes were purified through an anion exchange membrane chromatography process that comprised a 0.35M MgCl₂ excipient wash prior to product elution in a buffer comprised of 1.2M NaCl, pH 7.4. The AEX eluate was filtered through a 0.2 μm filter and spiked with 20 μM MgCl₂ and 10 U/mL of SAN. The bulk SAN-treated AEX eluate was sub-aliquoted and held at either 15° C. or 4° C. for 0, 1, 3, 7, or 14 days. Samples of each hold condition were placed at −80° C. and analyzed using qPCR immediately upon thaw. The digestion of DNA occurred quickly and reached a stable, consistent value whether in ambient (15° C.) or cold (4° C.) hold conditions (FIG. 18).

What is claimed is:

1. A method of preparing purified extracellular vesicles (EVs) from a sample comprising EVs and one or more nucleic acid molecules, comprising: (i) contacting the sample with a chromatography resin and (ii) contacting the chromatography resin with a nuclease wash buffer; wherein the nuclease wash buffer comprises a nuclease and a cation; and wherein the (ii) washing follows the (i) contacting.

2. A method of reducing the concentration of residual nucleic acid molecule in a sample comprising extracellular vesicles (EVs), comprising (i) contacting the sample with a chromatography resin and (ii) contacting the chromatography resin with a nuclease wash buffer; wherein the nuclease wash buffer comprises a nuclease and a cation; and wherein the (ii) washing follows the (i) contacting.

3. The method of claim 1 or 2, wherein the chromatography resin is selected from the group consisting of a cation exchange resin, an anion exchange (AEX) resin, an affinity chromatography resin, a pseudo affinity chromatography resin, a hydrophobic interaction resin, a hydrophobic charge induction chromatography resin, a mixed mode resin, an immobilized metal affinity resin, a ceramic hydroxyapatite resin, a fluoro hydroxyapatite resin, and any combination thereof.

4. The method of any one of claims 1 to 3, wherein the chromatography resin comprises an AEX resin.

5. The method of any one of claims 1 to 3, wherein the chromatography resin comprises a CEX resin.

6. The method of any one of claims 1 to 3, wherein the chromatography resin comprises an affinity chromatography resin.

7. The method of any one of claims 1 to 6, wherein the nuclease is an endonuclease.

8. The method of any one of claims 1 to 6, wherein the nuclease is an exonuclease.

9. The method of any one of claims 1 to 8, wherein the nuclease is selected from salt active nuclease (SAN), benzonase, denarase, kryptonase, and any combination thereof.

10. The method of any one of claims 1 to 7, wherein the nuclease comprises SAN.

11. The method of any one of claims 1 to 10, wherein the cation comprises a monovalent cation.

12. The method of claim 11, wherein the monovalent cation is selected from the group consisting of Li⁺, K⁺, Na⁺, NH4+, Cu⁺, and any combination thereof.

13. The method of any one of claims 1 to 12, wherein the cation comprises a divalent cation.

14. The method of claim 11, wherein the divalent cation is selected from the group consisting of Ca²⁺, Mg²⁺, Co²⁺, Ni²⁺, Zn²⁺, Ba²⁺, Sr²⁺, Al²⁺, Ag²⁺, Cu²⁺, Mn²⁺, and any combination thereof.

15. The method of claim 13 or 14, wherein the divalent cation comprises Mg²⁺.

16. The method of any one of claims 1 to 10, wherein the cation is a buffer selected from the group consisting of a Imidazole, Tris, TAPS, BisTRIS, arginine, histidine, lysine buffer, and any combination thereof.

17. The method of any one of claims 1 to 16, wherein the nuclease wash buffer further comprises an anion.

18. The method of claim 17, wherein the anion is selected from SCN, Cl⁻, SO₄⁻, PO₄⁻, Br⁻, I⁻, and any combination thereof.

19. The method of claim 17, wherein the anion is a buffer selected from the group consisting of a HEPES, BES, Bicine, MES, MOPS, PIPES, acetate, carbonate, citrate, bicarbonate buffer, aspartic acid, glutamic acid, and any combination thereof.

20. The method of any one of claims 1 to 19, wherein the cation is associated with an anion, wherein the association is selected from MgCl₂, Mg(SCN)₂, Mg(SO₄)₂, Mg(PO₄)₂, and any combination thereof.

21. The method of any one of claims 1 to 20, wherein the nuclease wash buffer comprises MgCl₂.

22. The method of any one of claims 1 to 21, wherein the sample is contacted with the chromatography resin in a loading buffer, wherein the loading buffer comprises a salt concentration from at least about 0.01 M to at least about 2.0 M.

23. The method of claim 22, wherein the salt concentration of the loading buffer is at least about 0.01 M, at least about 0.05 M, at least about 0.1 M, at least about 0.15 M, at least about 0.2 M, at least about 0.25 M, at least about 0.3 M, at least about 0.35 M, at least about 0.4 M, at least about 0.45 M, at least about 0.5 M, at least about 0.55 M, at least about 0.6 M, at least about 0.65 M, at least about 0.7 M, at least about 0.75 M, at least about 0.8 M, at least about 0.85 M, at least about 0.9 M, at least about 0.95 M, or at least about 1.0 M.

24. The method of claim 22 or 23, wherein the salt of the loading buffer is selected from NaCl, KCl, KPO₄, NaPO₄, CaCl₂), Mg₂SO₄, ZnCl₂, MnCl₂, MnSO₄, NaSCN, KSCN, LiCl, MgCl₂, and any combination thereof.

25. The method of claim 22, wherein the salt of the loading buffer comprises NaCl.

26. The method of claim 25, wherein the loading buffer comprises at least about 0.01 M NaCl, at least about 0.05 M NaCl, at least about 0.1 M NaCl, at least about 0.15 M NaCl, at least about 0.2 M NaCl, at least about 0.25 M NaCl, at least about 0.3 M NaCl, at least about 0.35 M NaCl, at least about 0.4 M NaCl, at least about 0.45 M NaCl, at least about 0.5 M NaCl, at least about 0.55 M NaCl, at least about 0.6 M NaCl, at least about 0.65 M NaCl, at least about 0.7 M NaCl, at least about 0.75 M NaCl, at least about 0.8 M NaCl, at least about 0.85 M NaCl, at least about 0.9 M NaCl, at least about 0.95 M NaCl, at least about 1 M NaCl, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M or at least about 2 M.

27. The method of any one of claims 22 to 26, wherein the loading buffer comprises at least about 0.55 M NaCl.

28. The method of any one of claims 1 to 27, wherein the nuclease wash buffer comprises at least about 1 unit/mL to at least about 100 units/mL, at least about 1 unit/mL to at least about 75 units/mL, at least about 1 unit/mL to at least about 50 units/mL, at least about 10 units/mL to at least about 100 units/mL, at least about 10 units/mL to at least about 75 units/mL, at least about 10 units/mL to at least about 50 units/mL, at least about 20 units/mL to at least about 100 units/mL, at least about 20 units/mL to at least about 75 units/mL, at least about 20 units/mL to at least about 50 units/mL, at least about 30 units/mL to at least about 100 units/mL, at least about 30 units/mL to at least about 75 units/mL, at least about 30 units/mL to at least about 50 units/mL, at least about 40 units/mL to at least about 100 units/mL, at least about 40 units/mL to at least about 75 units/mL, at least about 40 units/mL to at least about 50 units/mL, at least about 50 units/mL to at least about 100 units/mL, or at least about 50 units/mL to at least about 75 units/mL of the nuclease.

29. The method of any one of claims 1 to 28, wherein the nuclease wash buffer comprises at least about 1 unit/mL, at least about 5 units/mL, at least about 10 units/mL, at least about 15 units/mL, at least about 20 units/mL, at least about 25 units/mL, at least about 30 units/mL, at least about 35 units/mL, at least about 40 units/mL, at least about 45 units/mL, at least about 50 units/mL, at least about 60 units/mL, at least about 65 units/mL, at least about 70 units/mL, at least about 80 units/mL, at least about 90 units/mL, or at least about 100 unit/mL of the nuclease.

30. The method of any one of claims 1 to 29, wherein the nuclease wash buffer comprises at least about 1 unit/mL to at least about 100 units/mL SAN.

31. The method of any one of claims 1 to 30, wherein the nuclease wash buffer comprises at least about 1 unit/mL, at least about 5 units/mL, at least about 10 units/mL, at least about 15 units/mL, at least about 20 units/mL, at least about 25 units/mL, at least about 30 units/mL, at least about 35 units/mL, at least about 40 units/mL, at least about 45 units/mL, at least about 50 units/mL, at least about 60 units/mL, at least about 65 units/mL, at least about 70 units/mL, at least about 80 units/mL, at least about 90 units/mL, or at least about 100 unit/mL SAN.

32. The method of any one of claims 1 to 31, wherein the nuclease wash buffer comprises at least about 40 units/mL SAN.

33. The method of any one of claims 1 to 31, wherein the nuclease wash buffer comprises at least about 0.01 M to at least about 1.0 M of the cation.

34. The method of claim 31, wherein the nuclease wash buffer comprises at least about 0.01 M, at least about 0.05 M, at least about 0.1 M, at least about 0.15 M, at least about 0.2 M, at least about 0.25 M, at least about 0.3 M, at least about 0.35 M, at least about 0.4 M, at least about 0.45 M, at least about 0.5 M, at least about 0.55 M, at least about 0.6 M, at least about 0.65 M, at least about 0.7 M, at least about 0.75 M, at least about 0.8 M, at least about 0.85 M, at least about 0.9 M, at least about 0.95 M, or at least about 1.0 M of the cation.

35. The method of any one of claims 1 to 34, wherein the cation comprises Mg²⁺, and wherein the concentration of the Mg²⁺ in the nuclease wash buffer is at least about 0.05 M, at least about 0.1 M, at least about 0.15 M, at least about 0.2 M, at least about 0.25 M, at least about 0.3 M, at least about 0.35 M, at least about 0.4 M, at least about 0.45 M, at least about 0.5 M, at least about 0.55 M, at least about 0.6 M, at least about 0.65 M, at least about 0.7 M, at least about 0.75 M, at least about 0.8 M, at least about 0.85 M, at least about 0.9 M, at least about 0.95 M, or at least about 1.0 M Mg²⁺.

36. The method of claim 35, wherein the concentration of the Mg²⁺ in the nuclease wash buffer is at least about 0.35 M Mg²⁺.

37. The method of any one of claims 1 to 36, wherein the nuclease wash buffer comprises at least about 0.35 M MgCl₂.

38. The method of any one of claims 1 to 37, wherein the nuclease wash buffer is contacted with the chromatography resin at least 2 times, at least 3 times, at least 4 times, or at least 5 times.

39. The method of any one of claims 1 to 38, further comprising washing the chromatography resin by contacting the chromatography resin with a wash buffer, wherein the wash buffer does not comprise a nuclease.

40. The method of claim 39, wherein the chromatography resin is contacted with the wash buffer:
(a) after (i) contacting the sample with a chromatography resin, and before (ii) contacting the chromatography resin with a nuclease wash buffer;
(b) after (ii) contacting the chromatography resin with a nuclease wash buffer; or
(c) both (a) and (b).

41. The method of claim 39 or 40, wherein the wash buffer comprises a salt at a concentration of at least about 0.01 M, at least about 0.05 M, at least about 0.1 M, at least about 0.15 M, at least about 0.2 M, at least about 0.25 M, at least about 0.3 M, at least about 0.35 M, at least about 0.4 M, at least about 0.45 M, at least about 0.5 M, at least about 0.55 M, at least about 0.6 M, at least about 0.65 M, at least about 0.7 M, at least about 0.75 M, at least about 0.8 M, at least about 0.85 M, at least about 0.9 M, at least about 0.95 M, or at least about 1.0 M.

42. The method of claim 41, wherein the salt in the wash buffer is selected from NaCl, KCl, KPO₄, NaPO₄, CaCl₂), Mg2SO4, ZnCl2, MnCl2, MnSO4, NaSCN, KSCN, LiCl, and any combination thereof.

43. The method of any one of claims 39 to 42, wherein the wash buffer comprises NaCl.

44. The method of any one of claims 39 to 43, wherein the wash buffer comprises at least about 0.01 M NaCl, at least about 0.05 M NaCl, at least about 0.1 M NaCl, at least about 0.15 M NaCl, at least about 0.2 M NaCl, at least about 0.25 M NaCl, at least about 0.3 M NaCl, at least about 0.35 M NaCl, at least about 0.4 M NaCl, at least about 0.45 M NaCl, at least about 0.5 M NaCl, at least about 0.55 M NaCl, at least about 0.6 M NaCl, at least about 0.65 M NaCl, at least about 0.7 M NaCl, at least about 0.75 M NaCl, at least about 0.8 M NaCl, at least about 0.85 M NaCl, at least about 0.9 M NaCl, at least about 0.95 M NaCl, or at least about 1 M NaCl.

45. The method of any one of claims 39 to 44, wherein the wash buffer comprises at least about 0.55 M NaCl.

46. The method of any one of claims 1 to 45, further comprising (iii) eluting the EVs from the chromatography resin by contacting the chromatography resin with an elution buffer, wherein (iii) occurs after (ii) contacting the chromatography resin with a nuclease wash buffer.

47. The method of claim 46, wherein the elution buffer comprises a salt concentration of at least about 1.0 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2.0 M, at least about 2.5 M, at least about 3.0 M, at least about 3.5 M, at least about 4.0 M, at least about 4.5 M, or at least about 5.0 M.

48. The method of claim 46 or 47, wherein the elution buffer comprises a salt concentration of at least about 1.0 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2.0 M, at least about 2.5 M, at least about 3.0 M, at least about 3.5 M, at least about 4.0 M, at least about 4.5 M, or at least about 5.0 M NaCl.

49. The method of any one of claims 46 to 48, wherein the elution buffer comprises at least about 1.2 M NaCl.

50. The method of any one of claims 46 to 49, wherein the elution buffer releases one or more EVs from the chromatography resin.

51. The method of any one of claims 46 to 50, further comprising collecting an eluent after contacting the chromatography resin with the elution buffer.

52. The method of claim 51, wherein the eluent comprises one or more EVs.

53. The method of claim 51 or 52, wherein the sample contacted with the chromatography resin comprises a starting concentration of the one or more nucleic acid molecules, and wherein the eluent comprises an eluted concentration of the one or more nucleic acid molecules, wherein the eluted concentration of the one or more nucleic acid molecules is less than about 10%, less than about 5%, less than about 1%, less than about 0.5%, less than about 0.1%, less than about 0.05%, less than about 0.01%, less than about 0.001%, or less than about 0.0001% that of the starting concentration of the one or more nucleic acid molecules.

54. The method of any one of claims 1 to 53, further comprising subjecting the sample to one or more additional chromatography resins.

55. The method of claim 54, wherein the one or more additional chromatography resins comprises an anion exchange chromatography (AEX) resin, a cation exchange chromatography (CEX) resin, a mixed mode chromatography (MMC) resin, hydrophobic charge induction chromatography resin, a hydrophobic interaction chromatography resin, an immobilized metal affinity chromatography (IMAC), or any combination thereof.

56. The method of any one of claims 4 and 7 to 55, wherein the sample is contacted with a CEX resin after the AEX resin.

57. The method of claim 56, wherein the sample is contacted with an MMC resin after the CEX resin.

58. The method of any one of claims 4 and 7 to 55, wherein the sample is contacted with an MMC resin after the AEX resin.

59. The method of claim 58, wherein the sample is contacted with a CEX resin, an affinity resin, an HIC, a ceramic hydroxyapatite, a CFT, an IMAC, or any combination thereof after the MMC resin.

60. The method of any one of claims 1 to 59, wherein the sample is contacted with the chromatography resin and/or the additional chromatography resin at least two times, at least three times, at least four times, at least five times, at least six times, at least seven times, at least eight times, at least nine times, at least eight times, at least nine times, at least ten times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, or at least 25 times.

61. The method of any one of claims 1 to 60, wherein the sample is contacted with:
a. an AEX resin;
b. an CEX resin;
c. an MMC resin;
d. an affinity chromatography resin;
e. an HIC resin;
f. a ceramic hydroxyapatite resin;
g. an IMAC resin;
h. an HCIC resin; or
i. any combination thereof.

62. The method of any one of claims 1 to 61, wherein the EV is an exosome.

63. A composition comprising extracellular vesicles prepared by the method of any one of claims 1 to 60.

64. The composition of claim 63, which further comprises:
a. a saccharide,
b. sodium chloride,
c. a potassium phosphate,
d. a sodium phosphate, and
e. any combination thereof.

65. A method of treating a disease or condition in a subject in need thereof comprising administering the composition of claim 63 or 64.

66. A method of preparing purified extracellular vesicles (EVs) from a sample comprising EVs and one or more nucleic acid molecules, comprising: (i) contacting the sample with a chromatography resin and (ii) contacting the chromatography resin with a wash buffer comprising a nuclease, a cation, or both a nuclease and a cation; and wherein the (ii) washing follows the (i) contacting.

67. A method of reducing the concentration of residual nucleic acid molecule in a sample comprising extracellular vesicles (EVs), comprising (i) contacting the sample with a chromatography resin and (ii) contacting the chromatography resin with a wash buffer comprising a nuclease, a cation, or both a nuclease and a cation; and wherein the (ii) washing follows the (i) contacting.

68. A method of preparing purified extracellular vesicles (EVs) from a sample comprising EVs and one or more nucleic acid molecules, comprising: (i) contacting the sample with a chromatography resin; (ii) contacting the chromatography resin with a wash buffer comprising a nuclease, a cation, or both a nuclease and a cation; (iii) eluting an eluent from the chromatography resin, wherein the eluent comprises the EVs, e.g., exosomes.

69. The method of any one of claims 66 to 68, wherein the wash buffer comprises a nuclease (“a nuclease wash buffer”).

70. The method of any one of claims 66 to 69, wherein the wash buffer comprises a cation.

71. The method of any one of claims 66 to 71, further comprising (iv) contacting the eluent with a nuclease wash buffer.

72. A method of preparing purified extracellular vesicles (EVs) from a sample comprising EVs and one or more nucleic acid molecules, comprising: (i) contacting the sample with a chromatography resin, (ii) eluting an eluent form the chromatography resin, wherein the eluent comprises the EVs, e.g., exosomes, and (iii) contacting the eluent with a nuclease wash buffer.

73. A method of reducing the concentration of residual nucleic acid molecule in a sample comprising extracellular vesicles (EVs), comprising (i) contacting the sample with a chromatography resin, (ii) eluting an eluent form the chromatography resin, wherein the eluent comprises the EVs, e.g., exosomes, and (iii) contacting the eluent with a nuclease wash buffer.

74. The method of any one of claims 72 to 74, wherein the nuclease wash buffer comprises a nuclease and a cation.

75. The method of any one of claims 66 to 75, wherein the chromatography resin is selected from the group consisting of a cation exchange resin, an anion exchange (AEX) resin, an affinity chromatography resin, a pseudo affinity chromatography resin, a hydrophobic interaction resin, a hydrophobic charge induction chromatography resin, a mixed mode resin, an immobilized metal affinity resin, a ceramic hydroxyapatite resin, a fluoro hydroxyapatite resin, and any combination thereof.

76. The method of any one of claims 66 to 76, wherein the chromatography resin comprises an AEX resin.

77. The method of any one of claims 66 to 76, wherein the chromatography resin comprises a CEX resin.

78. The method of any one of claims 66 to 76, wherein the chromatography resin comprises an affinity chromatography resin.

79. The method of any one of claims 66 to 79, wherein the nuclease is an endonuclease.

80. The method of any one of claims 66 to 79, wherein the nuclease is an exonuclease.

81. The method of any one of claims 66 to 81, wherein the nuclease is selected from salt active nuclease (SAN), benzonase, denarase, kryptonase, and any combination thereof.

82. The method of any one of claims 66 to 82, wherein the nuclease comprises SAN.

83. The method of any one of claims 66 to 83, wherein the cation comprises a monovalent cation.

84. The method of claim 84, wherein the monovalent cation is selected from the group consisting of Li⁺, K⁺, Na⁺, NH4+, Cu⁺, and any combination thereof.

85. The method of any one of claims 66 to 85, wherein the cation comprises a divalent cation.

86. The method of claim 86, wherein the divalent cation is selected from the group consisting of Ca²⁺, Mg²⁺, Co²⁺, Ni²⁺, Zn²⁺, Ba²⁺, Sr²⁺, Al²⁺, Ag²⁺, Cu²⁺, Mn²⁺, and any combination thereof.

87. The method of claim 86 or 87, wherein the divalent cation comprises Mg²⁺.

88. The method of any one of claims 66 to 83, wherein the cation is a buffer selected from the group consisting of a Imidazole, Tris, TAPS, BisTRIS, arginine, histidine, lysine buffer, and any combination thereof.

89. The method of any one of claims 66 to 89, wherein the nuclease wash buffer further comprises an anion.

90. The method of claim 90, wherein the anion is selected from SCN, Cl⁻, SO₄⁻, PO₄⁻, Br⁻, I⁻, and any combination thereof.

91. The method of claim 90, wherein the anion is a buffer selected from the group consisting of a HEPES, BES, Bicine, MES, MOPS, PIPES, acetate, carbonate, citrate, bicarbonate buffer, aspartic acid, glutamic acid, and any combination thereof.

92. The method of any one of claims 66 to 91, wherein the cation is associated with an anion, wherein the association is selected from MgCl₂, Mg(SCN)₂, Mg(SO₄)₂, Mg(PO₄)₂, and any combination thereof.

93. The method of any one of claims 66 to 92, wherein the nuclease wash buffer comprises MgCl₂.

94. The method of any one of claims 66 to 93, wherein the sample is contacted with the chromatography resin in a loading buffer, wherein the loading buffer comprises a salt concentration from at least about 0.01 M to at least about 2.0 M.

95. The method of claim 94, wherein the salt concentration of the loading buffer is at least about 0.01 M, at least about 0.05 M, at least about 0.1 M, at least about 0.15 M, at least about 0.2 M, at least about 0.25 M, at least about 0.3 M, at least about 0.35 M, at least about 0.4 M, at least about 0.45 M, at least about 0.5 M, at least about 0.55 M, at least about 0.6 M, at least about 0.65 M, at least about 0.7 M, at least about 0.75 M, at least about 0.8 M, at least about 0.85 M, at least about 0.9 M, at least about 0.95 M, or at least about 1.0 M.

96. The method of claim 94 or 95, wherein the salt of the loading buffer is selected from NaCl, KCl, KPO₄, NaPO₄, CaCl₂), Mg2SO4, ZnCl2, MnCl2, MnSO4, NaSCN, KSCN, LiCl, MgCl₂, and any combination thereof.

97. The method of claim 96, wherein the salt of the loading buffer comprises NaCl.

98. The method of claim 97, wherein the loading buffer comprises at least about 0.01 M NaCl, at least about 0.05 M NaCl, at least about 0.1 M NaCl, at least about 0.15 M NaCl, at least about 0.2 M NaCl, at least about 0.25 M NaCl, at least about 0.3 M NaCl, at least about 0.35 M NaCl, at least about 0.4 M NaCl, at least about 0.45 M NaCl, at least about 0.5 M NaCl, at least about 0.55 M NaCl, at least about 0.6 M NaCl, at least about 0.65 M NaCl, at least about 0.7 M NaCl, at least about 0.75 M NaCl, at least about 0.8 M NaCl, at least about 0.85 M NaCl, at least about 0.9 M NaCl, at least about 0.95 M NaCl, at least about 1 M NaCl, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M or at least about 2 M.

99. The method of any one of claims 95 to 98, wherein the loading buffer comprises at least about 0.55 M NaCl.

100. The method of any one of claims 66 to 99, wherein the nuclease wash buffer comprises at least about 0.001 unit/mL to at least about 1000 units/mL, at least about 1 unit/mL to at least about 100 units/mL, at least about 1 unit/mL to at least about 75 units/mL, at least about 1 unit/mL to at least about 50 units/mL, at least about 10 units/mL to at least about 100 units/mL, at least about 10 units/mL to at least about 75 units/mL, at least about 10 units/mL to at least about 50 units/mL, at least about 20 units/mL to at least about 100 units/mL, at least about 20 units/mL to at least about 75 units/mL, at least about 20 units/mL to at least about 50 units/mL, at least about 30 units/mL to at least about 100 units/mL, at least about 30 units/mL to at least about 75 units/mL, at least about 30 units/mL to at least about 50 units/mL, at least about 40 units/mL to at least about 100 units/mL, at least about 40 units/mL to at least about 75 units/mL, at least about 40 units/mL to at least about 50 units/mL, at least about 50 units/mL to at least about 100 units/mL, or at least about 50 units/mL to at least about 75 units/mL of the nuclease.

101. The method of any one of claims 66 to 100, wherein the nuclease wash buffer comprises at least about 1 unit/mL, at least about 5 units/mL, at least about 10 units/mL, at least about 15 units/mL, at least about 20 units/mL, at least about 25 units/mL, at least about 30 units/mL, at least about 35 units/mL, at least about 40 units/mL, at least about 45 units/mL, at least about 50 units/mL, at least about 60 units/mL, at least about 65 units/mL, at least about 70 units/mL, at least about 80 units/mL, at least about 90 units/mL, or at least about 100 unit/mL of the nuclease.

102. The method of any one of claims 66 to 101, wherein the nuclease wash buffer comprises at least about 1 unit/mL to at least about 100 units/mL SAN.

103. The method of any one of claims 66 to 102, wherein the nuclease wash buffer comprises at least about 1 unit/mL, at least about 5 units/mL, at least about 10 units/mL, at least about 15 units/mL, at least about 20 units/mL, at least about 25 units/mL, at least about 30 units/mL, at least about 35 units/mL, at least about 40 units/mL, at least about 45 units/mL, at least about 50 units/mL, at least about 60 units/mL, at least about 65 units/mL, at least about 70 units/mL, at least about 80 units/mL, at least about 90 units/mL, or at least about 100 unit/mL SAN.

104. The method of any one of claims 66 to 103, wherein the nuclease wash buffer comprises at least about 40 units/mL SAN.

105. The method of any one of claims 66 to 104, wherein the nuclease wash buffer comprises at least about 0.01 M to at least about 1.0 M of the cation.

106. The method of claim 105, wherein the nuclease wash buffer comprises at least about 0.01 M, at least about 0.05 M, at least about 0.1 M, at least about 0.15 M, at least about 0.2 M, at least about 0.25 M, at least about 0.3 M, at least about 0.35 M, at least about 0.4 M, at least about 0.45 M, at least about 0.5 M, at least about 0.55 M, at least about 0.6 M, at least about 0.65 M, at least about 0.7 M, at least about 0.75 M, at least about 0.8 M, at least about 0.85 M, at least about 0.9 M, at least about 0.95 M, or at least about 1.0 M of the cation.

107. The method of any one of claims 66 to 106, wherein the cation comprises Mg²⁺, and wherein the concentration of the Mg²⁺ in the nuclease wash buffer is at least about 0.05 M, at least about 0.1 M, at least about 0.15 M, at least about 0.2 M, at least about 0.25 M, at least about 0.3 M, at least about 0.35 M, at least about 0.4 M, at least about 0.45 M, at least about 0.5 M, at least about 0.55 M, at least about 0.6 M, at least about 0.65 M, at least about 0.7 M, at least about 0.75 M, at least about 0.8 M, at least about 0.85 M, at least about 0.9 M, at least about 0.95 M, or at least about 1.0 M Mg²⁺.

108. The method of claim 107, wherein the concentration of the Mg²⁺ in the nuclease wash buffer is at least about 0.35 M Mg²⁺.

109. The method of any one of claims 66 to 108, wherein the nuclease wash buffer comprises at least about 0.35 M MgCl₂.

110. The method of any one of claims 66 to 72 and 75 to 109, wherein the nuclease wash buffer is contacted with the chromatography resin at least 2 times, at least 3 times, at least 4 times, or at least 5 times.

111. The method of any one of claims 72 to 110, wherein the eluent is contacted with the nuclease wash buffer by incubation for at least about 1 minute, at least about 2 minutes, at least about 3 minutes, at least about 4 minutes, at least about 5 minutes, at least about 10 minutes, at least about 15 minutes, at least about 20 minutes, at least about 25 minutes, at least about 30 minutes, at least about 60 minutes, at least about 90 minutes, at least about 120 minutes, at least about 180 minutes, at least about 3 hours, at least about 4 hours, at least about 5 hours, at least about 6 hours, at least about 8 hours, at least about 9 hours, at least about 10 hours, at least about 11 hours, at least about 12 hours, at least about 15 hours, at least about 18 hours, at least about 21 hours, or at least about 24 hours.

112. The method of any one of claims 72 to 110, wherein the eluent is contacted with the nuclease wash buffer by incubation for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days.

113. The method of any one of claims 72 to 112, wherein the eluent is contacted with the nuclease wash buffer by incubation at about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., or about 37° C.

114. The method of any one of claims 72 to 113, wherein the eluent is contacted with the nuclease wash buffer by incubation at about 4° C.

115. The method of any one of claims 72 to 113, wherein the eluent is contacted with the nuclease wash buffer by incubation at about 37° C.

116. The method of any one of claims 66 to 72, further comprising (iii) eluting the EVs from the chromatography resin, wherein (iii) occurs after (ii) contacting the chromatography resin with the wash buffer.

117. The method of any one of claims 73 to 116, wherein the EVs are eluted from the chromatography resin by contacting the chromatography resin with an elution buffer.

118. The method of claim 117, wherein the elution buffer comprises a salt concentration of at least about 1.0 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2.0 M, at least about 2.5 M, at least about 3.0 M, at least about 3.5 M, at least about 4.0 M, at least about 4.5 M, or at least about 5.0 M.

119. The method of claim 117 or 118, wherein the elution buffer comprises a salt concentration of at least about 1.0 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2.0 M, at least about 2.5 M, at least about 3.0 M, at least about 3.5 M, at least about 4.0 M, at least about 4.5 M, or at least about 5.0 M NaCl.

120. The method of any one of claims 117 to 119, wherein the elution buffer comprises at least about 1.2 M NaCl.

121. The method of any one of claims 117 to 120, wherein the elution buffer releases one or more EVs from the chromatography resin.

122. The method of any one of claims 73 to 121, wherein the sample contacted with the chromatography resin comprises a starting concentration of the one or more nucleic acid molecules, and wherein the eluent comprises an eluted concentration of the one or more nucleic acid molecules, wherein the eluted concentration of the one or more nucleic acid molecules is less than about 10%, less than about 5%, less than about 1%, less than about 0.5%, less than about 0.1%, less than about 0.05%, less than about 0.01%, less than about 0.001%, or less than about 0.0001% that of the starting concentration of the one or more nucleic acid molecules.

123. The method of any one of claims 66 to 122, further comprising subjecting the sample to one or more additional chromatography resins.

124. The method of claim 123, wherein the one or more additional chromatography resins comprises an anion exchange chromatography (AEX) resin, a cation exchange chromatography (CEX) resin, a mixed mode chromatography (MMC) resin, hydrophobic charge induction chromatography resin, a hydrophobic interaction chromatography resin, an immobilized metal affinity chromatography (IMAC), or any combination thereof.

125. The method of any one of claims 76, 77, and 80 to 124, wherein the sample is contacted with a CEX resin after the AEX resin.

126. The method of claim 125, wherein the sample is contacted with an MMC resin after the CEX resin.

127. The method of any one of claims 76, 77, and 80 to 124, wherein the sample is contacted with an MMC resin after the AEX resin.

128. The method of claim 127, wherein the sample is contacted with a CEX resin, an affinity resin, an HIC, a ceramic hydroxyapatite, a CFT, an IMAC, or any combination thereof after the MMC resin.

129. The method of any one of claims 66 to 128, wherein the sample is contacted with the chromatography resin and/or the additional chromatography resin at least two times, at least three times, at least four times, at least five times, at least six times, at least seven times, at least eight times, at least nine times, at least eight times, at least nine times, at least ten times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, or at least 25 times.

130. The method of any one of claims 66 to 123, wherein the sample is contacted with:
a. an AEX resin;
b. an CEX resin;
c. an MMC resin;
d. an affinity chromatography resin;
e. an HIC resin;
f. a ceramic hydroxyapatite resin;
g. an IMAC resin;
h. an HCIC resin; or
i. any combination thereof.

131. The method of any one of claims 66 to 132, wherein the EV is an exosome.

132. The method of any one of claims 1 to 62 and 66 to 131, wherein the concentration of EVs in the sample is at least about 1E9 to about 1E14 p/mL.
